(19) United States
(12) Patent Application Publication
(10) Pub. No.: US 2024/0417707 A1
(43) Pub. Date: Dec. 19, 2024

(54) COMPOSITIONS AND METHODS FOR IMPROVED GENOME EDITING WITH NME2CAS9 AND NME2-SMUCAS9 VARIANTS

(71) Applicant: UNIVERSITY OF MASSACHUSETTS, Boston, MA (US)

(72) Inventors: Erik Joseph Sontheimer, Auburndale, MA (US); Nathan Bamidele, Shrewsbury, MA (US); Zexiang Chen, Worcester, MA (US)

(21) Appl. No.: 18/660,923

(22) Filed: May 10, 2024

Related U.S. Application Data
(60) Provisional application No. 63/465,582, filed on May 11, 2023.

(51) Int. Cl. C12N 9/22; C12N 9/78; C12N 15/11; C12N 15/86; C12N 15/90

(52) U.S. Cl. CPC C12N 9/22; C12N 9/78; C12N 15/11; C12N 15/86; C12N 15/907; C12Y 305/04001; C12Y 305/04002; C07K 2319/09; C12N 2310/20; C12N 2750/14143

(57) ABSTRACT

The present disclosure relates to Neisseria meningitidis (Nme) 2 Cas9 (Nme2Cas9) and Nme2^{Smu}Cas9 variants comprising one or more amino acid substitutions with increased genome editing activities (e.g., improve nuclease and base editing efficiencies).

### CROSS REFERENCE TO RELATED APPLICATION

This application claims the benefit of U.S. Provisional Patent Application Ser. No. 63/465,582, filed May 11, 2023. The entire contents of the above-referenced patent application is incorporated by reference in their entirety herein.

### STATEMENT REGARDING FEDERALLY SPONSORED RESEARCH OR DEVELOPMENT

This invention was made with government support under GM143879 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML file, created on Aug. 26, 2024, is named 751969_UM9-302_ST26.xml and is 215,313 bytes in size.

### FIELD OF THE INVENTION

The disclosure relates to compositions and methods for genome editing with Nme2Cas9 and Nme2^{smu}Cas9 variants.

### BACKGROUND

Genome editing using CRISPR-Cas9 technologies has advanced genetic research and promises to revolutionize gene therapy. This includes nuclease editing which relies on DNA double strand breaks, in addition to alternative editing modalities such as base and prime editing. Most CRISPR-Cas9 gene editing technologies rely on efficient Cas9 nucleic acid binding, and/or cleavage and nicking of DNA strands at a genomic locus specified by the protospacer adjacent motif (PAM) and guide RNA. Type IIC Cas9 orthologues such as Nme2Cas9 and SmuCas9 often recognize favorable PAMs but are sometimes limited by their editing activity. This lower activity can sometimes result in limited efficacy for certain genome editing applications.

Accordingly, there exists a need in the art for Nme2Cas9 and Nme2^{Smu}Cas9 variants with increased genome editing activities in mammalian cells.

### SUMMARY

In one aspect, the disclosure provides a Neisseria meningitidis (Nme) 2 Cas9 (Nme2Cas9) variant comprising an amino acid substitution at one or more positions selected from the group consisting of E520, D873, D418, E471, D442, E844, E443, D470, E585, E552, D451, E587, E508, E932, D56, D1048, E1079, D660, E887, T72, and E186.

In certain embodiments, the Nme2Cas9 variant comprises 1, 2, 3, 4, or 5 amino acid substitutions.

In certain embodiments, the Nme2Cas9 variant comprises amino acid substitutions at positions E932 and D873; E932 and D56; E932 and E520; E932 and D1048; D873 and D56; D873 and E520; D873 and D1048; D56 and E520; D56 and D1048; E520 and D1048; E932, D873, and D56; E932, D873, and E520; E932, D873, and D1048; E932, D56, and E520; E932, D56, and D1048; E932, E520, and D1048; D873, D56, and E520; D873, D56, and D1048; D873, E520, and D1048; D56, E520, and D1048; E932, D873, D56, and E520; E932, D873, D56, and D1048; E932, D56, E520, and D1048; D873, D56, E520, and D1048; or E932, D873, D56, E520, and D1048.

In certain embodiments, the amino acid substitution is a positively charged amino acid. In certain embodiments, the amino acid substitution is an arginine (R), lysine (K), or histidine (H). In certain embodiments, the amino acid substitution is an arginine (R).

In certain embodiments, the Nme2Cas9 variant comprises an amino acid substitution of any one or more of E520R, D873R, D418R, E471R, D442R, E844R, E443R, D470R, E585R, E552R, D451R, E587R, E508R, E932R, D56R, D1048R, E1079R, D660R, E887R, T72R, and E186R.

In certain embodiments, the Nme2Cas9 variant comprises an amino acid substitution of any one or more of E520R, D873R, D418R, E471R, D442R, E844R, E443R, E932R, D56R, D1048R, E1079R, D660R, E887R, T72R, and E186R.

In certain embodiments, the Nme2Cas9 variant comprises amino acid substitutions E932R and D873R; E932R and D56R; E932R and E520R; E932R and D1048R; D873R and D56R; D873R and E520R; D873R and D1048R; D56R and E520R; D56R and D1048R; E520R and D1048R; E932R, D873R, and D56R; E932R, D873R, and E520R; E932R, D873R, and D1048R; E932R, D56R, and E520R; E932R, D56R, and D1048R; E932R, E520R, and D1048R; D873R, D56R, and E520R; D873R, D56R, and D1048R; D873R, E520R, and D1048R; D56R, E520R, and D1048R; E932R, D873R, D56R, and E520R; E932R, D873R, D56R, and D1048R; E932R, D56R, E520R, and D1048R; D873R, D56R, E520R, and D1048R; or E932R, D873R, D56R, E520R, and D1048R.

In certain embodiments, the Nme2Cas9 variant comprises a protospacer adjacent motif interacting domain (PID) that interacts with an N₄CC nucleotide sequence, an N₄CA nucleotide sequence, an N₄CG nucleotide sequence, an N₄CT nucleotide sequence, or an N₄C nucleotide sequence.

In certain embodiments, the PID is an Nme2Cas9 PID or an SmuCas9 PID.

In certain embodiments, the Nme2Cas9 PID comprises an amino acid sequence set forth in (DNGDMVRVDVFCKVDKKGKNQYFIVPIYAWQVAENILPDIDCKGYRIDDSYTFCFSL HKYDLIAFQKDEKSKVEFAYYINCDSSNGRFYLAWHDKGSKEQQFRISTQNLVLIQKY QVNELGKEIRPCRLKKRPPVR)(SEQ ID NO:27).

In certain embodiments, the SmuCas9 PID comprises an amino acid sequence set forth in (DNATMVRVDVYTKAGKNYLVPVYVWQVAQGILPNRAVTSGKSEADWDLIDESFEFK FSLSRGDLVEMISNKGRIFGYYNGLDRANGSIGIREHDLEKSKGKDGVHRVGVKTATA FNKYHVDPLGKEIHRCSSEPRPTLKIKSKK) (SEQ ID NO:28).

In certain embodiments, the one or more amino acid positions are relative to an amino acid sequence of SEQ ID NO: 1 or SEQ ID NO: 2.

In certain embodiments, the Nme2Cas9 variant further comprises a nucleotide base editor (NBE) domain fused to the Nme2Cas9 variant.

In certain embodiments, the NBE domain is an inlaid NBE domain inserted into the Nme2Cas9 variant.

In certain embodiments, the inlaid NBE domain is inserted into a recognition (REC) domain of the Nme2Cas9 variant.

In certain embodiments, the inlaid NBE domain is inserted into a HNH domain of the Nme2Cas9 variant.

In certain embodiments, the inlaid NBE domain is inserted into a RuvC domain of the Nme2Cas9 variant.

In certain embodiments, the inlaid NBE domain is inserted between amino acid position 291 and amino acid position 292 of the Nme2Cas9 variant, relative to an amino acid sequence of SEQ ID NO: 1 or 2.

In certain embodiments, the inlaid NBE domain is inserted between amino acid position 761 and amino acid position 762 of the Nme2Cas9 variant, relative to an amino acid sequence of SEQ ID NO: 1 or 2.

In certain embodiments, the inlaid NBE domain is inserted between amino acid position 795 and amino acid position 796 of the Nme2Cas9 variant, relative to an amino acid sequence of SEQ ID NO: 1 or 2.

In certain embodiments, the inlaid NBE domain is flanked at the inlaid NBE domain N-terminus and/or C-terminus by an amino acid linker.

In certain embodiments, the amino acid linker comprises a (GGS)ₙ(SEQ ID NO:40) linker, wherein n corresponds to 1-6.

In certain embodiments, the amino acid linker comprises GGSGGSGGSGGSGGSGGSGG(SEQ ID NO: 15).

In certain embodiments, the amino acid linker comprises GSSGSETPGTSESATPESSG(SEQ ID NO: 21).

In certain embodiments, the amino acid linker comprises ED.

In certain embodiments, the inlaid NBE domain is flanked at the inlaid NBE domain N-terminus by GGSGGSGGSGGSGGSGGSGG(SEQ ID NO: 15) and at the inlaid NBE domain C-terminus by GSSGSETPGTSESATPESSG(SEQ ID NO: 21).

In certain embodiments, the NBE domain is linked via an amino acid linker to the N-terminus of the Nme2Cas9 variant.

In certain embodiments, the NBE domain is linked via an amino acid linker to the C-terminus of the Nme2Cas9 variant.

In certain embodiments, the amino acid linker comprises a (GGS)ₙ(SEQ ID NO:40) linker, wherein n corresponds to 1-6.

In certain embodiments, the amino acid linker comprises GGSGGSGGSGGSGGSGGSGG(SEQ ID NO: 15).

In certain embodiments, the amino acid linker comprises GSSGSETPGTSESATPESSG(SEQ ID NO: 21).

In certain embodiments, the amino acid linker comprises ED.

In certain embodiments, the inlaid NBE domain is an adenine base editor (ABE) domain.

In certain embodiments, the inlaid ABE domain is an inlaid adenosine deaminase protein domain.

In certain embodiments, the inlaid adenosine deaminase protein domain is an adenosine deaminase 8e protein domain (TadA8e).

In certain embodiments, the TadA8e comprises an amino acid sequence set forth in (SEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNSKRGAA GSLMNVLNYPGMNHRVEITEGILADECAALLCDFYRMPRQVFNAQKKAQSSIN) (SEQ ID NO: 9).

In certain embodiments, the TadA8e comprises a V105W amino acid substitution relative to the amino acid sequence set forth in (SEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNSKRGAA GSLMNVLNYPGMNHRVEITEGILADECAALLCDFYRMPRQVFNAQKKAQSSIN) (SEQ ID NO: 9).

In certain embodiments, the TadA8e comprises an amino acid sequence set forth in (SEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGWRNSKRGAA GSLMNVLNYPGMNHRVEITEGILADECAALLCDFYRMPRQVFNAQKKAQSSIN) (SEQ ID NO:29).

In certain embodiments, the inlaid NBE domain is a cytidine base editor (CBE) domain.

In certain embodiments, the inlaid CBE domain is an inlaid cytosine deaminase protein domain.

In certain embodiments, the cytosine deaminase protein domain is evoFERNY or rAPOBEC1.

In certain embodiments, the evoFERNY comprises an amino acid sequence set forth in (FERNYDPRELRKETYLLYEIKWGKSGKLWRHWCQNNRTQHAEVYFLENIFNARRFNP STHCSITWYLSWSPCAECSQKIVDFLKEHPNVNLEIYVARLYYPENERNRQGLRDLVN SGVTIRIMDLPDYNYCWKTFVSDQGGDEDYWPGHFAPWIKQYSLKL) (SEQ ID NO: 13).

In certain embodiments, the rAPOBEC1 comprises an amino acid sequence set forth in (SSETGPVAVDPTLRRRIEPHEFEVFFDPRELRKETCLLYEINWGGRHSIWRHTSQNTNK HVEVNFIEKFTTERYFCPNTRCSITWFLSWSPCGECSRAITEFLSRYPHVTLFIYIARLYH HADPRNRQGLRDLISSGVTIQIMTEQESGYCWRNFVNYSPSNEAHWPRYPHLWVRLY VLELYCIILGLPPCLNILRRKQPQLTFFTIALQSCHYQRLPPHILWATGLK) (SEQ ID NO: 11).

In certain embodiments, the Nme2Cas9 variant further comprises one or more nuclear localization signals (NLS).

In certain embodiments, the one or more NLS are any one or more of a nucleoplasmin NLS, an SV40 NLS or a C-myc NLS.

In certain embodiments, the one or more NLS comprise an amino acid sequence selected from the group consisting of MKRTADGSEFESPKKKRKV(SEQ ID NO:30), KRTADGSEFEPKKKRKV(SEQ ID NO:31), MKRPAATKKAGQAKKKK(SEQ ID NO:32), KRPAATKKAGQAKKKK(SEQ ID NO:33), MPKKKRKV(SEQ ID NO:34), and PKKKRKV(SEQ ID NO:35).

In certain embodiments, the one or more NLS are positioned at the N-terminus and/or C-terminus of the Nme2Cas9 variant.

In certain embodiments, the Nme2Cas9 variant further comprises a uracil glycosylase inhibitor (UGI).

In certain embodiments, the Nme2Cas9 variant further comprises a D16A substitution.

In one aspect, the disclosure provides a polynucleotide encoding the Nme2Cas9 variant described herein.

In certain embodiments, the polynucleotide is a messenger RNA (mRNA).

In one aspect, the disclosure provides a vector comprising the polynucleotide sequence described herein.

In one aspect, the disclosure provides a viral vector comprising the polynucleotide sequence described herein.

In certain embodiments, the viral vector is an adeno-associated virus (AAV) vector or a lentiviral vector.

In one aspect, the disclosure provides an adeno-associated virus (AAV) comprising the polynucleotide sequence described herein.

In one aspect, the disclosure provides a genome editing system comprising the Nme2Cas9 variant described herein or a polynucleotide encoding the Nme2Cas9 variant described herein, and a guide RNA (gRNA).

In certain embodiments, the gRNA comprises: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence.

In certain embodiments, the gRNA comprises at least one modified nucleotide.

In certain embodiments, the at least one modified nucleotide comprises a modification of a ribose group, a phosphate group, a nucleobase, or a combination thereof.

In certain embodiments, the modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-0,4′-C-aminomethylene bridged nucleic acid (2′,4′-BNA^{NC}).

In certain embodiments, the modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, the modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the gRNA further comprises a nucleotide or non-nucleotide loop or linker linking the 3′ end of the crRNA portion to the 5′ end of the tracrRNA portion.

In certain embodiments, the nucleotide loop is chemically modified.

In certain embodiments, the nucleotide loop comprises the nucleotide sequence of GAAA.

In one aspect, the disclosure provides a method of editing a genome, comprising: (a) introducing into the genome the genome editing system described herein; and (b) incubating the genome editing system with the genome for a time sufficient to edit the genome.

In certain embodiments, the genome edit results from a single stranded and/or double strand DNA break.

In certain embodiments, the genome edit is a base edit.

In one aspect, the disclosure provides a fusion protein comprising a Neisseria meningitidis (Nme) 2 Cas9 (Nme2Cas9) protein and an inlaid nucleotide base editor (NBE) domain, wherein the inlaid NBE domain is flanked at the inlaid NBE domain N-terminus and/or C-terminus by an amino acid linker, or a linker is absent, and wherein the total number of amino acid linker residues is less than 40 amino acids.

In certain embodiments, the amino acid linker that is present at the N-terminus of the inlaid NBE domain is 20 amino acids long, and the amino acid linker that is present at the C-terminus of the inlaid NBE domain is 19 amino acids long, 18 amino acids long, 17 amino acids long, 16 amino acids long, 15 amino acids long, 14 amino acids long, 13 amino acids long, 12 amino acids long, 11 amino acids long, 10 amino acids long, 9 amino acids long, 8 amino acids long, 7 amino acids long, 6 amino acids long, 5 amino acids long, 4 amino acids long, 3 amino acids long, 2 amino acids long, 1 amino acid long, or is absent.

In certain embodiments, the amino acid linker that is present at the N-terminus of the inlaid NBE domain is 20 amino acids long, and the amino acid linker that is present at the C-terminus of the inlaid NBE domain is 10 amino acids long, 5 amino acids long, or is absent.

In certain embodiments, the amino acid linker that is present at the C-terminus of the inlaid NBE domain is 20 amino acids long, and the amino acid linker that is present at the N-terminus of the inlaid NBE domain is 19 amino acids long, 18 amino acids long, 17 amino acids long, 16 amino acids long, 15 amino acids long, 14 amino acids long, 13 amino acids long, 12 amino acids long, 11 amino acids long, 10 amino acids long, 9 amino acids long, 8 amino acids long, 7 amino acids long, 6 amino acids long, 5 amino acids long, 4 amino acids long, 3 amino acids long, 2 amino acids long, 1 amino acid long, or is absent.

In certain embodiments, the amino acid linker that is present at the C-terminus of the inlaid NBE domain is 20 amino acids long, and the amino acid linker that is present at the N-terminus of the inlaid NBE domain is 10 amino acids long, 5 amino acids long, or is absent.

In certain embodiments, the amino acid linker that is present at the N-terminus of the inlaid NBE domain is 10 amino acids long, and the amino acid linker that is present at the C-terminus of the inlaid NBE domain is 20 amino acids long, 19 amino acids long, 18 amino acids long, 17 amino acids long, 16 amino acids long, 15 amino acids long, 14 amino acids long, 13 amino acids long, 12 amino acids long, 11 amino acids long, 10 amino acids long, 9 amino acids long, 8 amino acids long, 7 amino acids long, 6 amino acids long, 5 amino acids long, 4 amino acids long, 3 amino acids long, 2 amino acids long, 1 amino acid long, or is absent.

In certain embodiments, the amino acid linker that is present at the N-terminus of the inlaid NBE domain is 10 amino acids long, and the amino acid linker that is present at the C-terminus of the inlaid NBE domain is 20 amino acids long, 10 amino acids long, 5 amino acids long, or is absent.

In certain embodiments, the amino acid linker that is present at the N-terminus of the inlaid NBE domain is 5 amino acids long, and the amino acid linker that is present at the C-terminus of the inlaid NBE domain is 20 amino acids long, 19 amino acids long, 18 amino acids long, 17 amino acids long, 16 amino acids long, 15 amino acids long, 14 amino acids long, 13 amino acids long, 12 amino acids long, 11 amino acids long, 10 amino acids long, 9 amino acids long, 8 amino acids long, 7 amino acids long, 6 amino acids long, 5 amino acids long, 4 amino acids long, 3 amino acids long, 2 amino acids long, 1 amino acid long, or is absent.

In certain embodiments, the amino acid linker that is present at the N-terminus of the inlaid NBE domain is 5 amino acids long, and the amino acid linker that is present at the C-terminus of the inlaid NBE domain is 20 amino acids long, 10 amino acids long, 5 amino acids long, or is absent.

In certain embodiments, the amino acid linker that is present at the N-terminus of the inlaid NBE domain is absent, and the amino acid linker that is present at the C-terminus of the inlaid NBE domain is 20 amino acids long, 19 amino acids long, 18 amino acids long, 17 amino acids long, 16 amino acids long, 15 amino acids long, 14 amino acids long, 13 amino acids long, 12 amino acids long, 11 amino acids long, 10 amino acids long, 9 amino acids long, 8 amino acids long, 7 amino acids long, 6 amino acids long, 5 amino acids long, 4 amino acids long, 3 amino acids long, 2 amino acids long, 1 amino acid long, or is absent.

In certain embodiments, the amino acid linker that is present at the N-terminus of the inlaid NBE domain is absent, and the amino acid linker that is present at the C-terminus of the inlaid NBE domain is 20 amino acids long, 10 amino acids long, 5 amino acids long, or is absent.

In certain embodiments, the amino acid linker comprises a sequence selected from the group consisting of: GGSGGSGGSGGSGGSGGSGG(SEQ ID NO: 15), SGGSGGSGGS(SEQ ID NO: 17), GGSGG(SEQ ID NO: 19), GSSGSETPGTSESATPESSG(SEQ ID NO: 21), ETPGTSESAT(SEQ ID NO: 23), and GTSES(SEQ ID NO: 25).

In certain embodiments, the amino acid linker that is present at the N-terminus of the inlaid NBE domain comprises GGSGGSGGSGGSGGSGGSGG(SEQ ID NO: 15), and the amino acid linker that is present at the C-terminus of the inlaid NBE domain comprises ETPGTSESAT(SEQ ID NO: 23), GTSES(SEQ ID NO: 25), or is absent.

In certain embodiments, the amino acid linker that is present at the N-terminus of the inlaid NBE domain comprises SGGSGGSGGS(SEQ ID NO: 17), and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker that is present at the N-terminus of the inlaid NBE domain comprises GGSGG(SEQ ID NO: 19), and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker is absent at the N-terminus of the inlaid NBE domain, and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker that is present at the C-terminus of the inlaid NBE domain comprises GSSGSETPGTSESATPESSG(SEQ ID NO: 21), and the amino acid linker that is present at the N-terminus of the inlaid NBE domain comprises SGGSGGSGGS(SEQ ID NO: 17), GGSGG(SEQ ID NO: 19), or is absent.

In certain embodiments, the amino acid linker that is present at the C-terminus of the inlaid NBE domain comprises ETPGTSESAT(SEQ ID NO: 23), and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

In certain embodiments, the amino acid linker that is present at the C-terminus of the inlaid NBE domain comprises GTSES(SEQ ID NO: 25), and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

In certain embodiments, the amino acid linker is absent at the C-terminus of the inlaid NBE domain, and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

In certain embodiments, the Nme2Cas9 comprises the Nme2Cas9 variant described herein.

In one aspect, the disclosure provides a polynucleotide encoding the fusion protein described herein.

In certain embodiments, the polynucleotide is a messenger RNA (mRNA).

In one aspect, the disclosure provides a vector comprising the polynucleotide sequence described herein.

In one aspect, the disclosure provides a viral vector comprising the polynucleotide sequence described herein.

In certain embodiments, the viral vector is an adeno-associated virus (AAV) vector or a lentiviral vector.

In one aspect, the disclosure provides an adeno-associated virus (AAV) comprising the polynucleotide sequence described herein.

These and other aspects of the applicant's teachings are set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects, features, benefits, and advantages of the embodiments described herein will be apparent with regard to the following description, examples, claims, and accompanying drawings where:

FIG. 1 presents an Nme2^{Smu}Cas9 homology model using the SWISS-MODEL server. Negatively charged amino acids (represented as spheres) within 5-10 angstroms of nucleic acid phosphate backbone were selected for Arginine mutagenesis. Spheres denote amino acids in close proximity to a corresponding nucleic acid. Red, target strand (TS) DNA; orange, sgRNA and blue, non-target strand (NTS) DNA.

FIGS. 2A-2B present exemplary embodiments of the ABE mCherry reporter.

FIG. 2A: Displays a schematic of the ABE mCherry reporter system for identifying gene editing activity such as a precise A to G conversion. The ABE reporter is stably integrated into the genome of HEK293T cells.

FIG. 2B: (SEQ ID NO(s):43-44), (SEQ ID NO:102) Displays the Nme2Cas9 N₄CN PAM target sites for activating the ABE mCherry reporter.

FIGS. 3A-3B present exemplary data of the Nme2^{Smu}-ABE-i1 arginine single mutants' activity at Target-Strand (TS) and non-target strand (NTS).

FIG. 3A: Displays the activities of Nme2^{Smu}-ABE8e-i1 (denoted as WT) and Target-Strand (TS) interacting arginine mutants (light grey bars) in the mCherry ABE reporter cell line (activated upon A-to-G editing). After plasmid transfection with an N₄CC PAM targeting sgRNA plasmid and a base editor plasmid, activities were measured by flow cytometry (n=2 biological replicates; data represent mean±SD).

FIG. 3B: Displays the activities of Nme2^{Smu}-ABE8e-i1 (denoted as WT), single guide RNA (SG) and non-target strand (NTS) interacting arginine mutants (light grey bars) in the mCherry ABE reporter cell line (activated aupon A-to-G editing). After plasmid transfection with an N₄CC PAM targeting sgRNA plasmid and a base editor plasmid, activities were measured by flow cytometry (n=3 biological replicates; data represent mean±SD).

FIGS. 4A-4B present exemplary data of the Nme2^{Smu}-ABE-arginine single mutants' activity at N₄CD PAM Targets.

FIG. 4A: Displays the activities of Nme2^{Smu}-ABE8e-i1, and top-performing arginine mutants in the mCherry ABE reporter cell line (activated upon A-to-G editing) at N₄CD (D not —C) PAM targets. After plasmid transfection with associated sgRNA plasmid and a base editor plasmid, activities were measured by flow cytometry (n=3 biological replicates; data represent mean±SD).

FIG. 4B: Displays the activities of Nme2^{Smu}-ABE8e-i1 in the mCherry ABE reporter compiled from the data above. Each data point represents the mean activity of a single PAM target site. Nme2^{Smu}-ABE8e-i1 mutants (grey bars), are ordered from best to worst performing with Nme2^{Smu}-ABE8e-i1 as a reference (blue bar).

FIG. 5: (SEQ ID NO(s):45-46), (SEQ ID NO:103) presents an illustration of the Nme2Cas9 N₄CN PAM target sites for mCherry Activation in the TLR-MCV1 reporter via nuclease mediated NHEJ.

FIGS. 6A-6B present data comparing the activities of four nuclease variants within the HEK293T TLR-MCV1 reporter at N₄CN PAM targets: Nme2Cas9, eNme2-C·NR, Nme2^{Smu}Cas9 and Nme2^{Smu}Cas9.

FIG. 6A: Displays the activities of four nuclease variants within the HEK293T TLR-MCV1 reporter at N₄CN PAM targets: Nme2Cas9, eNme2-C·NR, Nme2^{Smu}Cas9 and Nme2^{Smu}Cas9: wildtype Nme2^{Smu}Cas9 nuclease activity is denoted by the black line (solid), whereas eNme2-C·NR activity is denoted by the red line (dashed). After parallel plasmid transfection with associated sgRNA plasmid and a nuclease editor plasmid, activities were measured by flow cytometry (n=2 biological replicates; data represent mean±SD).

FIG. 6B: Displays the activities of four nuclease variants within the HEK293T TLR-MCV1 reporter at N₄CN PAM targets: Nme2Cas9, eNme2-C·NR, Nme2^{Smu}Cas9 and Nme2^{Smu}Cas9. Each data point represents the mean activity of a single PAM target site. Nme2^{Smu}Cas9 mutants are ordered from best to worst performing with Nme2Cas9 and Nme2^{Smu}Cas9 as references (WT and NmeCas9).

FIG. 7 displays the correlation between ABE and nuclease Nme2^{Smu}Cas9 effectors. Fold-changes in the observed activity of the top performing Nme2Smu Arginine mutations correlate for nuclease and ABE editing when compared to Wild-Type Nme2^{Smu}Cas9 (nuclease) or Nme2Smu-ABE8e-i1 (ABE) in the reporter assays.

FIGS. 8A-8B present Nme2^{Smu}Cas9 mutations for ABE/Nuclease.

FIG. 8A: Shows an Nme2^{Smu}Cas9 homology model using the SWISS-MODEL server. The Top 5 activating Arginine mutations and their locations are represented as colored speres. Spheres are color coded in respect to the nucleic acid they are in closest proximity too: Red, target DNA strand; orange, single guide RNA and blue, non-target DNA strand.

FIG. 8B: Shows a cartoon of an open reading frame (ORF, not drawn to scale) depicting the relative positions of top 5 arginine mutants (red asterisks) within Nme2^{Smu}Cas9.

FIGS. 9A-9C present data comparing the activities of five nuclease variants within the HEK293T TLR-MCV1 reporter at PAM targets: Nme2Cas9, eNme2-C·NR (vliu), eNme2-C·NR (vEJS), Nme2^{Smu}Cas9 and Nme2^{Smu}Cas9.

FIG. 9A: Shows data comparing the activities of five nuclease variants within the HEK293T TLR-MCV1 reporter at N₄CN PAM targets: Nme2Cas9, eNme2-C·NR (vliu), eNme2-C·NR (vEJS), Nme2^{Smu}Cas9 and Nme2^{Smu}Cas9 at N₄CN PAMs. Wildtype Nme2^{Smu}Cas9 nuclease activity is denoted by the black line (solid), whereas eNme2-C·NR (vEJS) and activity is denoted by the red line (dashed). After parallel plasmid transfection with associated sgRNA plasmid and a nuclease editor plasmid, activities were measured by flow cytometry (n=2 biological replicates; data represent mean±SD).

FIG. 9B: Shows the mean activity of a the Nme2^{Smu}Cas9 variants at N₄CN PAM targets. Each data point represents the mean activity of a single N₄CN PAM target site. Nme2^{Smu}Cas9 mutants (grey bars), are ordered from best to worst performing with Nme2Cas9, Nme2^{Smu}Cas9 and eNme2-C·NR as referencesNme2^{Smu}Cas9 mutants (light grey bars), which are ordered from best to worst performing with Nme2Cas9, Nme2^{Smu}Cas9 and eNme2-C·NR as references.

FIG. 9C: Shows the mean activity of the Nme2^{Smu}Cas9 variants at N₄CD PAM targets. Each data point represents the mean activity of a single PAM target site. Nme2^{Smu}Cas9 mutants (light grey bars), are ordered from best to worst performing with Nme2Cas9, Nme2^{Smu}Cas9 and eNme2-C·NR as references.

FIGS. 10A-10D show the A-to-G editing at four endogenous HEK293T genomic loci with Nme2^{smu}-ABE8e-i1 or Nme2^{smu}-ABE8e-i8 linker variant constructs by plasmid transfection.

FIG. 10A: Shows the A-to-G editing at four endogenous HEK293T genomic loci with Nme2^{Smu}-ABE8e-i1 linker variant constructs by plasmid transfection. Maximally edited adenine for each target site was plotted as a single data point and aggregated by linker variant. Editing activities measured by amplicon sequencing. n=3 biological replicates, data represent mean±SEM.

FIG. 10B: Displays the Max A-to-G editing rate of an individual N₄CC target site summarized. Each data point represents the Max A-to-G editing rate of an individual N₄CC target site summarized in (A), measured by amplicon sequencing. n=3 biological replicates. Data represent mean±SEM.

FIG. 10C: Displays nuclease editing at endogenous HEK293T genomic loci with Nme2Cas9 or Nme2^{smu}Cas9 constructs by plasmid transfection. Editing activities measured by amplicon sequencing. n=3 biological replicates, data represent mean±SD.

FIG. 10D: Displays represents nuclease editing rate of an individual N₄CC target site. Each data point represents nuclease editing rate of an individual N₄CC target site summarized in (C), measured by amplicon sequencing. n=3 biological replicates. Data represent mean±SEM.

FIGS. 11A-11C display a schematic of the Domain-Inlaid Nme2^{Smu}-ABE's.

FIG. 11A: Shows a schematic of AAV9 Nme2-ABE-i1 with a size of approximately 4.9 kb.

FIG. 11B: Shows a schematic of a domain-inlaid Nme2^{Smu}-ABE.

FIG. 11C: Denotes combinations of N-terminal and C-Terminal linkers flanking the TadA8e deaminase domain for size minimized Nme2^{Smu}-ABE-i1 transgenes. For example, the original Nme2^{Smu}-ABE-i1 transgene has 20 amino acid linkers flanking each side of deaminase (N-term linker, N-20) and (C-term linker, C-20).

FIGS. 12A-12B display A-to-G editing at four endogenous HEK293T genomic loci with Nme2^{smu}-ABE8e-i1 or Nme2^{smu}-ABE8e-i8 linker variant constructs by plasmid transfection.

FIG. 12A: Displays A-to-G editing at four endogenous HEK293T genomic loci with Nme2^{Smu}-ABE8e-i1 linker variant constructs by plasmid transfection. Maximally edited adenine for each target site was plotted as a single data point and aggregated by linker variant. Editing activities measured by amplicon sequencing. n=3 biological replicates, data represent mean±SEM.

FIG. 12B: Displays A-to-G editing at four endogenous HEK293T genomic loci with Nme2^{Smu}-ABE8e-i8 linker variant constructs by plasmid transfection. Maximally edited adenine for each target site was plotted as a single data point and aggregated by linker variant. Editing activities measured by amplicon sequencing. n=3 biological replicates, data represent mean±SEM.

FIGS. 13A-13B display the editing windows of Nme2^{Smu}-ABE-i1 and Nme2^{Smu}-ABE-i8 linker variants tested at four endogenous N₄CN PAM Targets in HEK293T. A-to-G conversion for each variant was normalized on a scale of 0-100 (%) against adenine positions with the highest observed edited efficiencies within the window of target sites tested. n=3 biological replicates.

FIG. 13A: Displays the editing windows of Nme2^{Smu}-ABE-i1 linker variants tested at four endogenous N₄CN PAM Targets in HEK293T. A-to-G conversion for each variant was normalized on a scale of 0-100 (%) against adenine positions with the highest observed edited efficiencies within the window of target sites tested. n=3 biological replicates.

FIG. 13B: Displays the editing windows of Nme2^{Smu}-ABE-i8 linker variants tested at four endogenous N₄CN PAM Targets in HEK293T. A-to-G conversion for each variant was normalized on a scale of 0-100 (%) against adenine positions with the highest observed edited efficiencies within the window of target sites tested. n=3 biological replicates.

FIGS. 14A-14H present the characterization of the activity and specificity of Nme2- and Nme2^{Smu}Cas9 nuclease variants.

FIG. 14A: Displays nuclease-induced indels in experimental panel 2 of the guide-target activity library following plasmid transfection of Nme2Cas9 (WT and E932R, D56R variants), Nme2^{Smu}Cas9 (WT and E932R, D56R, and E520R/D873R variants) or eNme2-C·NR into HEK293T cells with integrated guide-target sites with N₄CN PAMs. The editing efficiencies for 190 target sites were plotted.

FIG. 14B: Displays nuclease-induced indels in experimental panel 1 of the guide-target activity library following plasmid transfection of Nme2Cas9 (WT), Nme2^{Smu}Cas9 (WT and E932R, D56R, and E520R/D873R variants) or eNme2-C·NR into HEK293T cells. The editing efficiencies for 173 target sites were plotted. Editing activities were measured by amplicon sequencing (n=3 biological replicates; Boxplots represent median and interquartile range; whiskers indicate 5th and 95th percentiles and the cross represents the mean).

FIG. 14C: Displays averaged indel frequencies of Nme2^{Smu}Cas9 or eNme2-C·NR across single-(S) or di-nucleotide (D) mismatched target sites within the guide-target mismatch library. Activities for each mismatched target were normalized to the activity of their respective perfectly matched target site. Orange nucleotides represent protospacer position of the transversion mutation present within the mismatched target site.

FIG. 14D: Displays bulk indel frequencies of the nuclease variants within the mismatch library for: 12 perfectly matched target sites (0 MM), 252 single-(1 MM) or 204 double-(2 MM), mismatched target sites.

FIG. 14E: Displays indel vs. specificity scores for Nme2^{Smu}Cas9 variants or eNme2-C·NR across the mismatched guide-target library. Indel efficiency was compiled data from the 12 perfectly matched target sites (0 MM) in (FIG. 14C). The specificity score was calculated as, one minus the tiled mismatched editing mean in (FIG. 14B) normalized to a scale of one to 100. Data were measured by amplicon sequencing (n=3 biological replicates; Boxplots represent median and interquartile ranges; whiskers indicate 5th and 95th percentiles and the cross represents the mean).

FIG. 14F: Displays a table showing 40 mm targets (per guide) with constant A8, A12, and A15 for the design of a NmeCase9 library. The library is for nuclease/ABE specificity assays and has the following features: (1) comprises 480 members (12 guides X 40 targets); (2) the library member targets have constant A8, A12, and A15 to enable editing of ABE within their window; (3) the breakdown for each guide in the library is the following (40 targets/guide): (a) 2 perfect match guide —targets (MMO); (b) 21 single mm [transversion] (S1-S21); and (c) 17 double mm [transversion] (D1-D17); (4) the library is based on Tol2 Transposon Integration System; (5) the library member targets are tested with nuclease and ABE editors; and (6) targets sites are synthetic and based on highly active sites.

FIG. 14G: Shows a cartoon of an open reading frame (ORF) depicting the relative position of the guide.

FIG. 14H: : (SEQ ID NO(s):47-70) Displays a table of mismatch library targets. The library is for nuclease/ABE specificity assays and has the following features: (1) comprises 480 members (12 guides X 40 targets); and (2) 12 guides perfectly matched guides-target pairs, wherein: (a) per the possible targets in the variable 6th position of the N₄CN PAM, three pam targets were designed respectively.; (b) these target sites are synthetic (not present within the genome), and based on previously validated human genomic target sites; and (c) in protospacer positions 8, 12 and 15 of the target sites mentioned in (b), adenines (lowercase a), were manually added in place of the wildtype sequence.

FIGS. 15A-15B present the specificity characterization of Nme2- and Nme2^{Smu}Cas9 nucleases at N₄CC PAM targets.

FIG. 15A: Displays the indel editing frequencies of Nme2Cas9, Nme2^{Smu}Cas9 variants and eNme2-C·NR across single-(S) or di-nucleotide (D) mismatched target sites within the guide-target mismatch library. Activities for each mismatched target were normalized to the mean efficiency of their respective perfectly matched target site. Orange nucleotides represent protospacer position of the transversion mutation present within the mismatched target site.

FIG. 15B: Displays the indel activity vs. specificity score for nuclease variants in (a) across the mismatched guide-target library. Nuclease editing data for the three N₄CC perfectly matched target sites (0 MM). The specificity score was calculated as, one minus the tiled mismatched editing mean in (a) normalized to a scale of one to 100. Editing activities were measured by amplicon sequencing (n=3 biological replicates).

FIGS. 16A-16G present the characterization of the editing window and activity of domain-inlaid Nme2^{Smu}-ABE8e variants.

FIG. 16A: Displays a table depicting rAAV genome size in bp for respective domain-inlaid editors with linker variants and associated regulatory elements (right). Regulatory elements for all-in-one AAV packaging include ITRs, Ula promoter, ABE8e editor, U6 promoter and sgRNA cassette.

FIG. 16B: Displays cartoon schematics depicting open reading frame length (in bp) of domain-inlaid Nme2-ABE8e with Nme2Cas9 PID (left, top) or Nme2Smu-ABE8e with the SmuCas9 PID (left, bottom) with 20AA linkers flanking N- and C-termini of Tad8e.

FIG. 16C: Displays the assessment of editing windows and activities from experimental panel 2 of the guide-target activity library (183 sites) for Nme2^{Smu}-ABE8e-i1 or -i8, arginine mutants (E932R, D56R, E520R/D873R) in combination with deaminase linker lengths (L20, L10, L5). Following plasmid transfection of the ABE variants into HEK293T cells with the integrated guide-target library, editing activities were measured by amplicon sequencing. Left: average editing windows across the target sites, normalized on a scale of 0-100 (%) against adenine positions with the highest observed edited efficiencies within the window. Right: activities at the maximally edited adenine for each target were plotted (n=3 biological replicates; boxplots represent median and interquartile ranges; whiskers indicate 5th and 95th percentiles and the cross represents the mean).

FIG. 16D: Displays summary data from self-targeting library maximal activity, aggregated from (FIG. 16A). The Nme2^{Smu}-ABE8e and arginine mutant activity independent of domain insertion site and linker length is displayed.

FIG. 16E: Displays summary data from self-targeting library maximal activity, aggregated from (FIG. 16A). The Nme2^{Smu}-ABE8e and arginine mutant activity by position of domain insertion is displayed.

FIG. 16F: Displays summary data from self-targeting library maximal activity, aggregated from (FIG. 16A). The Nme2^{Smu}-ABE8e and linker variant activity independent of domain insertion site and arginine mutation is displayed.

FIG. 16G: Displays summary data from self-targeting library maximal activity, aggregated from (FIG. 16A). The Nme2^{Smu}-ABE8e and linker variant activity by position of domain insertion is displayed.

FIGS. 17A-17C present the specificity characterization of domain-inlaid Nme2^{Smu}-ABE8e variants.

FIG. 17A: Displays mean A-to-G editing efficiency across the targets within the mismatch library for domain-inlaid Nme2^{Smu}-ABE8e variants or eNme2-C. Data was subset by number of mismatches between guide and target site: 12 perfectly matched sites (0 MM), 252 single mismatched sites (1 MM) and 204 double-mismatched sites (2 MM). Each data point represents the average A-to-G editing observed across a protospacer of an individual library member.

FIG. 17B: Displays mean A-to-G editing frequencies of domain-inlaid Nme2^{Smu}-ABE8e variants or eNme2-C across single-(S) or di-nucleotide (D) mismatched target sites within the guide-target mismatch library. Activities for each mismatched target were normalized to the mean efficiency of their respective perfectly matched target site. Orange nucleotides represent protospacer position of the transversion mutation present within the mismatched target site.

FIG. 17C: Displays ABE activity vs. specificity scores for base editing variants in (FIG. 17A and FIG. 17B) across the mismatched guide-target library. ABE activity was compiled from editing data for perfectly matched target sites (0 MM) in (FIG. 17A). The specificity score was calculated as, one minus the tiled mismatched editing mean in (FIG. 17B) normalized to a scale of one to 100. Data were measured by amplicon sequencing (n=3 biological replicates; Boxplots represent median and interquartile ranges; whiskers indicate 5th and 95th percentiles and the cross represents the mean).

FIGS. 18A-18C present the characterization of the activity and editing window of engineered Nme2Cas9 variants in various ABE8e formats. The assessment of editing activities and windows from experimental panel 4 of the guide-target activity library (181 target sites) for Nme2-, Nme2^{Smu}-, iNme2-, iNme2^{Smu}- and eNme2-C variants in either the n-terminal and inlaid-i1 (linker 10) format were performed.

FIG. 18A: Displays the efficiency at the maximally edited adenine for each target that was plotted for all N₄CN PAM target sites. ABEs with a WT Nme2Cas9 PID (WT PID) or N₄CN targeting PID (single-C PID) are depicted by color.

FIG. 18B: Displays mean A-to-G editing activities and editing windows across protospacer positions in the activity guide-target library for engineered Nme2-ABE8e variants in the domain-inlaid-i1 (linker 10) format.

FIG. 18C: Displays data in (FIG. 18A) subset by target site PAM identity (N₄CC, N₄CT, N₄CG, N₄CA) for the engineered Nme2-ABE8e variants in the domain-inlaid-i1 (linker 10) format. The maximally edited adenine for each target was plotted. n in graph represents the number of target sites per PAM. (n=3 biological replicates; boxplots represent median and interquartile ranges; whiskers indicate 5th and 95th percentiles and the cross represents the mean).

FIGS. 19A-19B present a summary of editing windows and genomic targetable adenines by various Nme2Cas9-derived ABEs.

FIG. 19A: Displays summary editing windows of Nme2^{Smu}-ABE8e-i1 or Nme2^{Smu}-ABE8e-i8 with the L10 linker format and E932R mutation. The data represents the normalized editing rates across the window from three independent self-targeting library experimental panels, compiled from FIG. 17A and FIG. 18A. Each experimental panel consisted of 3 biological replicates.

FIG. 19B: Displays adenines targetable within the hg38 reference genome by Nme2Cas9-derived ABE8e variants in various formats. Editing windows to calculate the targetable adenines within the reference genome consisted of the previously described window for N-terminally fused Nme2-ABE8e(Davis et al., 2022), or the editing windows observed here with the guide-target library assay for N-terminally fused eNme2-C or domain-inlaid-i1 or -i8 Nme2^{Smu}-ABE8e editors from (FIG. 19A). Targetable adenine calculations were also made for whether the ABE uses dinucleotide (N₄CC) or single nucleotide cytidine (N₄CN) PAMs. Activity above 75% of the maximum position in the window was the cutoff criteria for window selection. Code used to generate this data was adapted from Davis et al. 2022 (Davis et al., 2022).

FIGS. 20A-20B present the specificity characterization of domain-inlaid Nme2- and Nme2^{Smu}-ABE8e variants at N₄CC PAM targets.

FIG. 20A: Displays mean A-to-G editing frequencies of domain-inlaid Nme2^{Smu}-ABE8e variants or eNme2-C across single-(S) or di-nucleotide (D) mismatched target sites within the guide-target mismatch library. Activities for each mismatched target were normalized to the mean efficiency of their respective perfectly matched target site. Orange nucleotides represent protospacer position of the transversion mutation present within the mismatched target site.

FIG. 20B: Displays ABE activity vs. specificity score for base editing variants in (a) across the mismatched guide-target library. ABE activity was compiled from editing data for three perfectly matched N₄CC target sites (0 MM). The specificity score was calculated as, one minus the tiled mismatched editing mean in (a) normalized to a scale of one to 100. Editing activities were measured by amplicon sequencing (n=3 biological replicates).

FIG. 21 presents the editing window characterization of domain inlaid Nme2^{Smu}-ABEs with narrow-window adenine deaminases. Assessment of editing windows and activities from experimental panel 4 of the guide-target activity library (193 sites) for narrow window deaminases (ABE8e, or ABE9e). Test subjects include Nme2^{Smu}-ABE-i1 or -i8, Arginine mutants (E932R, D56R) in combination with deaminase linker lengths (L10 and L5) or eNme2-C. Following plasmid transfection of the ABE variants into Hek293T cells with the integrated guide-target library; left—shows average editing windows across the target sites, normalized on a scale of 0-100 (%) against adenine positions with the highest observed edited efficiencies within the window. Right—the maximally edited adenine for each target was plotted. Editing activities were measured by amplicon sequencing. (n=3 biological replicates; boxplots represent median and interquartile ranges; whiskers indicate 5th and 95th percentiles and the cross represents the mean).

FIGS. 22A-22B present the activity and editing window characterization of domain-inlaid Nme2- and Nme2^{Smu}-ABE8e variants at N₄CC or N₄CN PAM targets. Assessment of editing windows and activities from experimental panel 3 of the guide-target activity library (192 sites) for Nme2-, Nme2^{Smu}-ABE8e-i1 or -i8, and arginine mutants (E932R, D56R), in combination with the L10 deaminase linker, as well as eNme2-C.

FIG. 22A: Displays subset of data focusing on editing windows and activities for N₄CC PAM targets only (49 sites). Following plasmid transfection of the ABE variants into HEK293T cells with the integrated guide-target library, editing activities were measured by amplicon sequencing. Left: average editing windows across the target sites, normalized on a scale of 0-100 (%) against adenine positions with the highest observed edited efficiencies within the window. Right: efficiency at the maximally edited adenine for each target was plotted.

FIG. 22B: Displays subset of data at N₄CD PAM target sites, for domain inlaid Nme2- or Nme2^{Smu}-ABE8e-i1 editors. The maximally edited adenine for each target was plotted. n in graph represents the number of target sites per PAM. (n=3 biological replicates; boxplots represent median and interquartile ranges; whiskers indicate 5th and 95th percentiles and the cross represents the mean).

FIG. 23 presents the activity and editing window characterization of domain-inlaid Nme2- and Nme2^{Smu}-ABE8e variants at N₄CN PAM targets. Assessment of editing windows and activities from experimental panel 4 of the guide-target activity library (181 N₄CN PAM sites) for Nme2-, Nme2^{Smu}-, iNme2-, iNme2^{Smu}- and eNme2-C variants in either the n-terminal and inlaid-i1 (linker 10) formats. Mean A-to-G editing activities and editing windows across protospacer positions in the activity guide-target library for the engineered Nme2Cas9 ABE8e variants. Editing activities were measured by amplicon sequencing (n=3 biological replicates).

FIG. 24 presents the Activity and editing window characterization of domain-inlaid Nme2- and Nme2^{Smu}-ABE8e variants at N₄CC PAM targets. Assessment of editing windows and activities from experimental panel 4 of the guide-target activity library (38 N₄CC PAM sites) for Nme2-, Nme2^{Smu}-, iNme2-, iNme2^{Smu}- and eNme2-C variants in either the n-terminal and inlaid-i1 (linker 10) formats. Mean A-to-G editing activities and editing windows across protospacer positions in the activity guide-target library for the engineered Nme2Cas9 ABE8e variants. Editing activities were measured by amplicon sequencing (n=3 biological replicates).

FIGS. 25A-25V present nucleotide sequences of Nme2Cas9 and Nme2^{Smu}Cas9 base editors.

FIG. 25A: (SEQ ID NO:71) Displays the nucleotide sequence of Nme2-ABE8e-nt: BPSV40-NLS, Nme2Cas9, TadA8e, Linkers.

FIG. 25B: (SEQ ID NO:72) Displays the nucleotide sequence of Nme2-ABE8e−i1: BPSV40-NLS, Nme2Cas9, TadA8e, Linkers.

FIG. 25C: (SEQ ID NO:73) Displays the nucleotide sequence of Nme2-ABE8e-i2: BPSV40-NLS, Nme2Cas9, TadA8e, Linkers.

FIG. 25D: (SEQ ID NO:74) Displays the nucleotide sequence of Nme2-ABE8e-i3: BPSV40-NLS, Nme2Cas9, TadA8e, Linkers.

FIG. 25E: (SEQ ID NO:75) Displays the nucleotide sequence of Nme2-ABE8e-i4: BPSV40-NLS, Nme2Cas9, TadA8e, Linkers.

FIG. 25F: (SEQ ID NO:76) Displays the nucleotide sequence of Nme2-ABE8e-i5: BPSV40-NLS, Nme2Cas9, TadA8e, Linkers.

FIG. 25G: (SEQ ID NO:77) Displays the nucleotide sequence of Nme2-ABE8e-i6: BPSV40-NLS, Nme2Cas9, TadA8e, Linkers.

FIG. 25H: (SEQ ID NO:78) Displays the nucleotide sequence of Nme2-ABE8e-i7: BPSV40-NLS, Nme2Cas9, TadA8e, Linkers.

FIG. 251: (SEQ ID NO:79) Displays the nucleotide sequence of Nme2-ABE8e-i8: BPSV40-NLS, Nme2Cas9, TadA8e, Linkers.

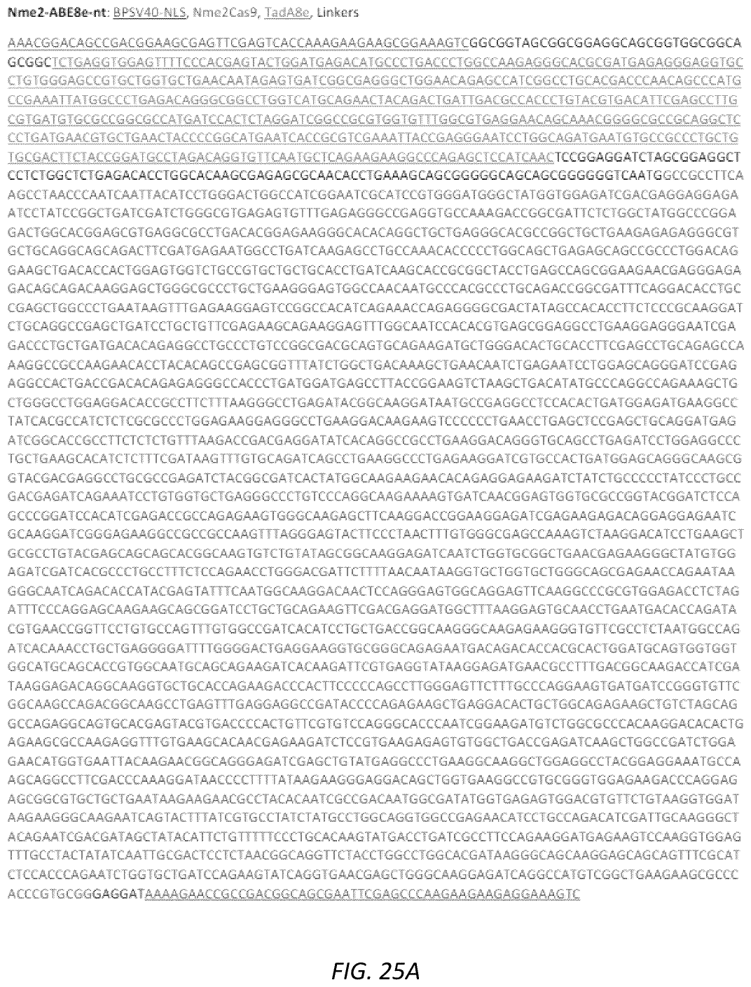

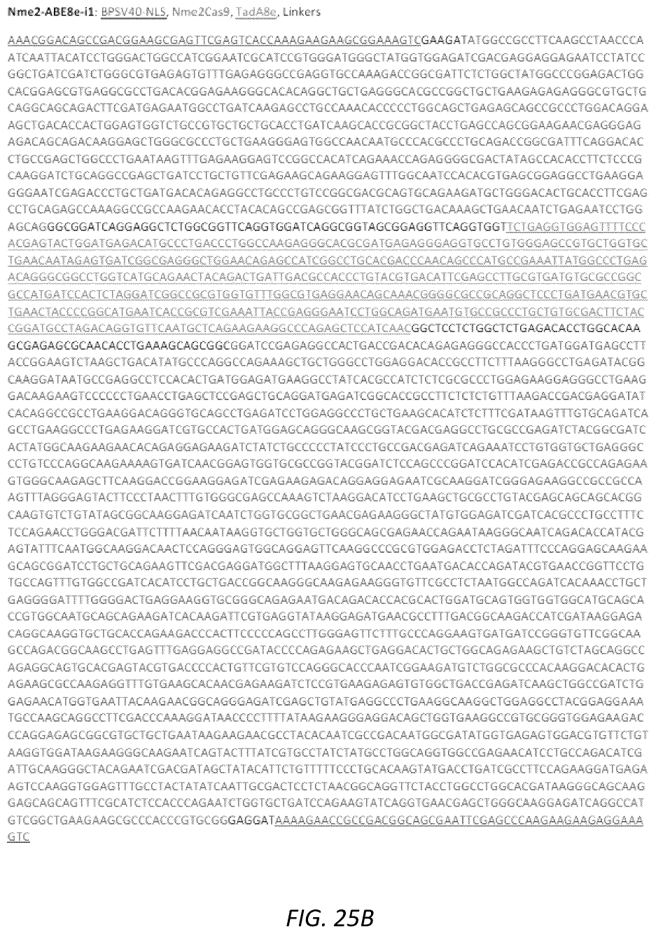

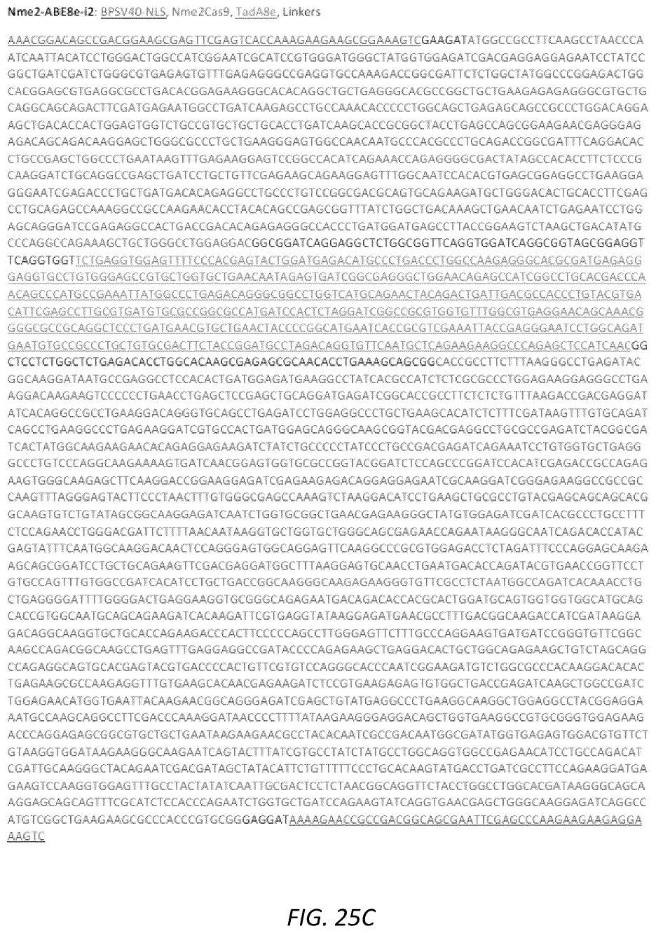

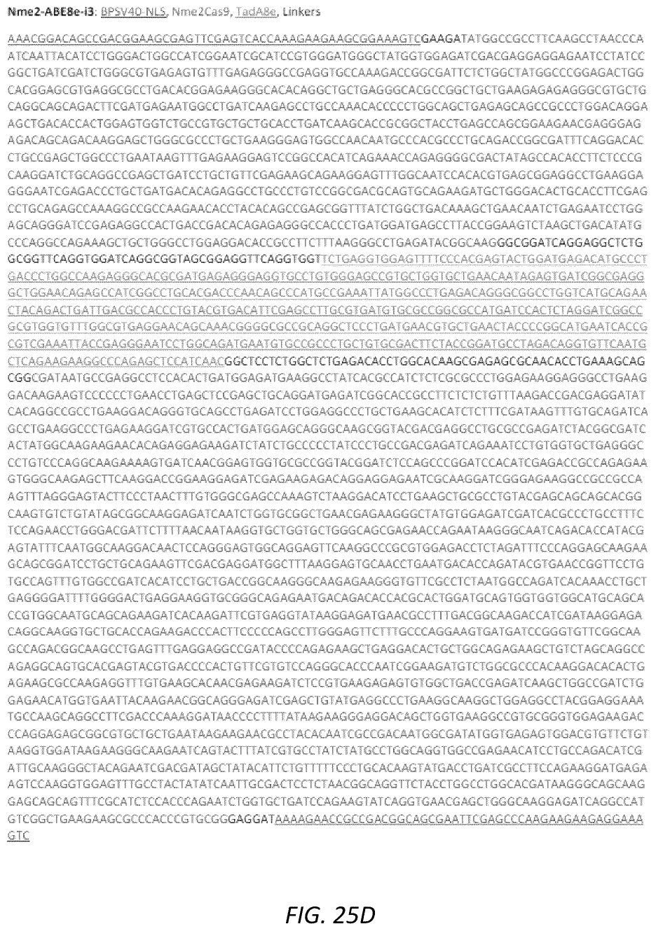

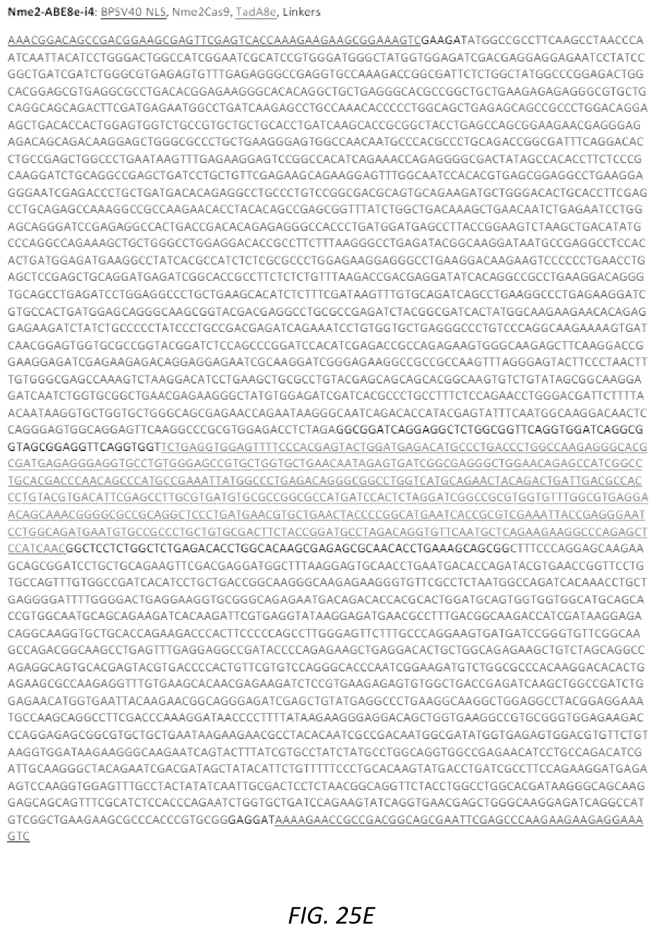

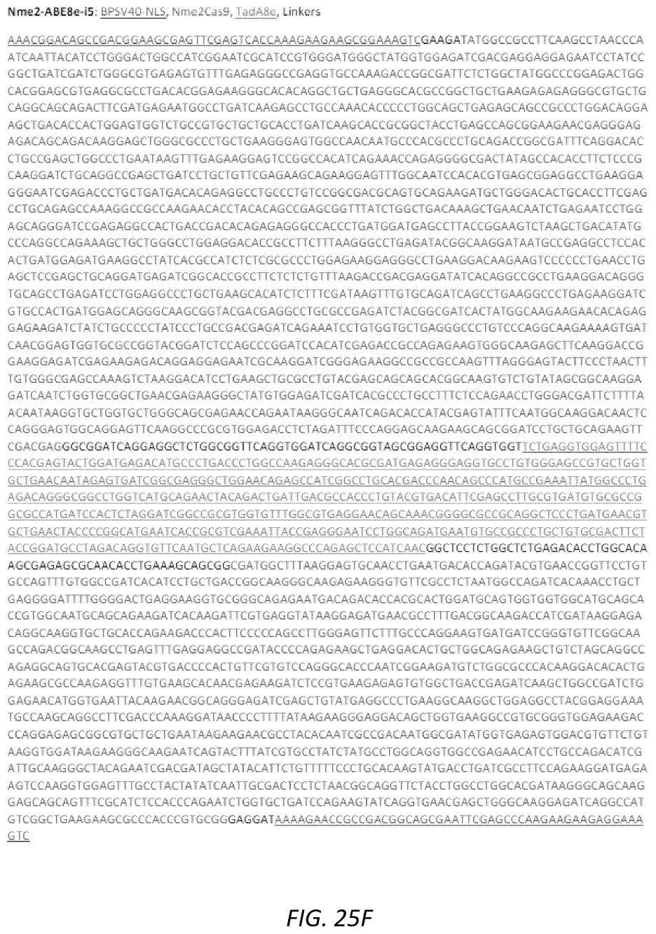

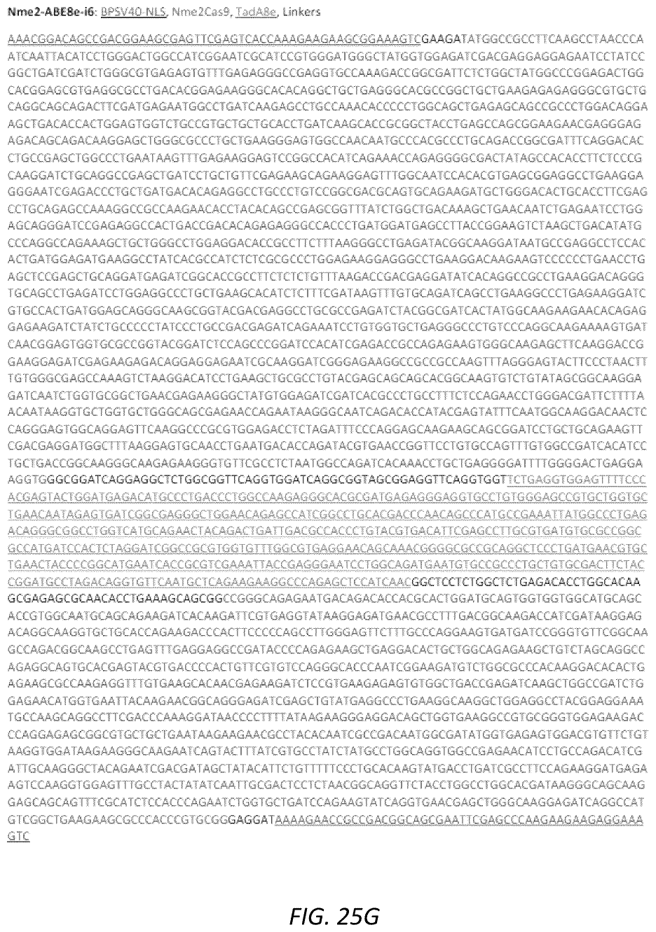

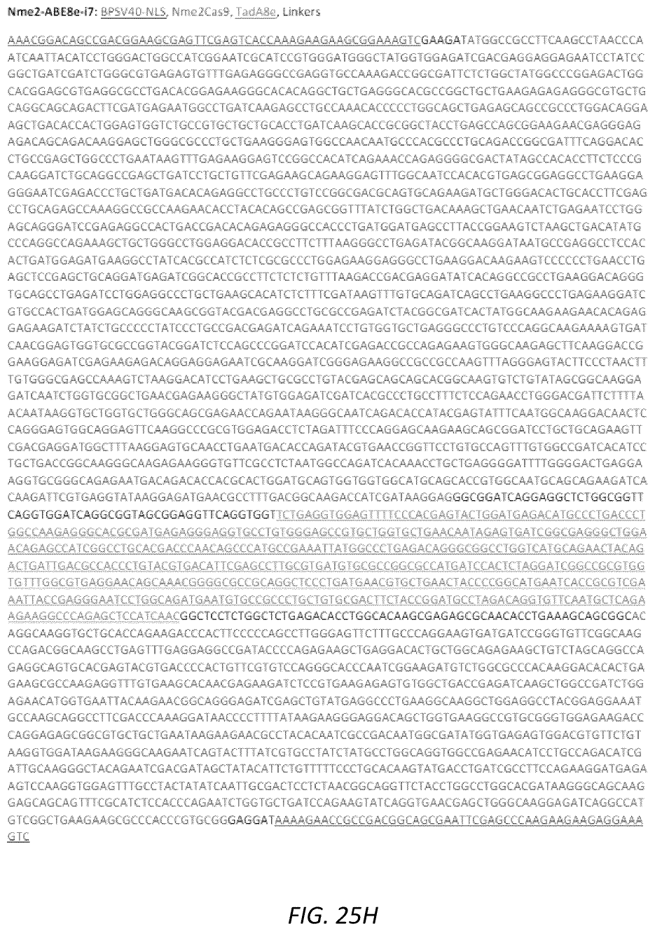

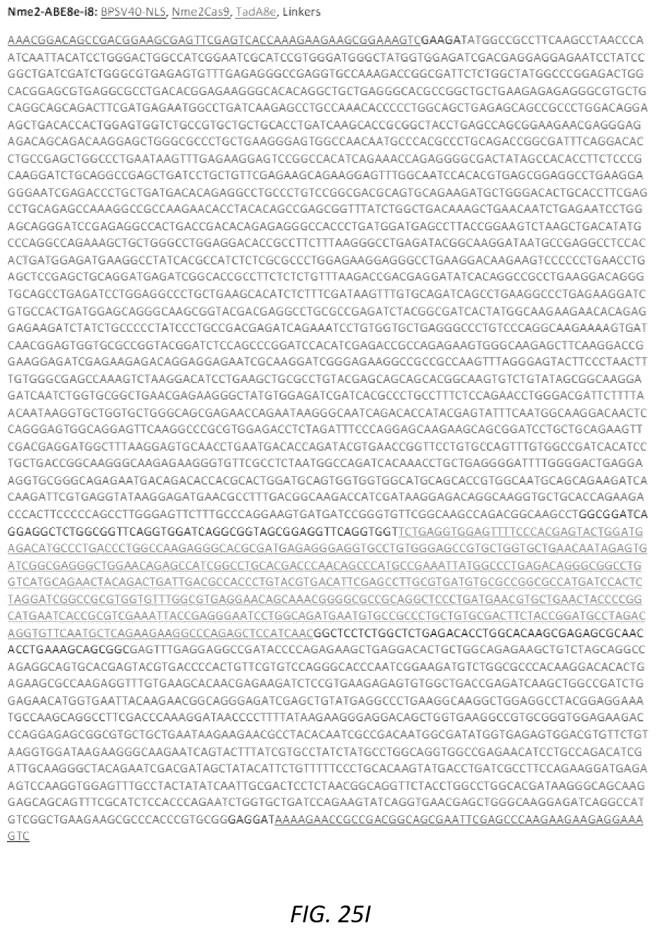

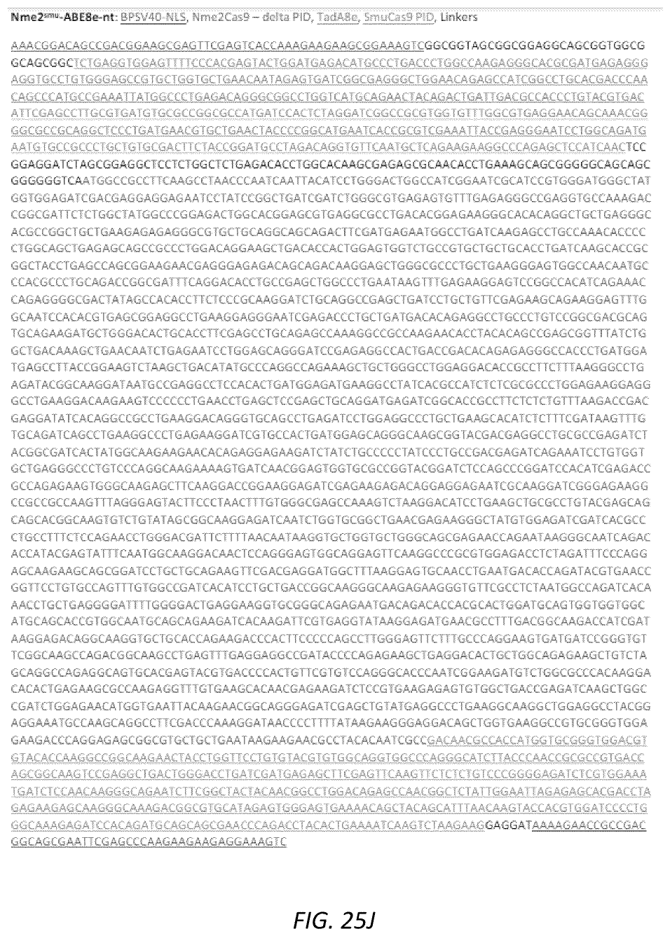

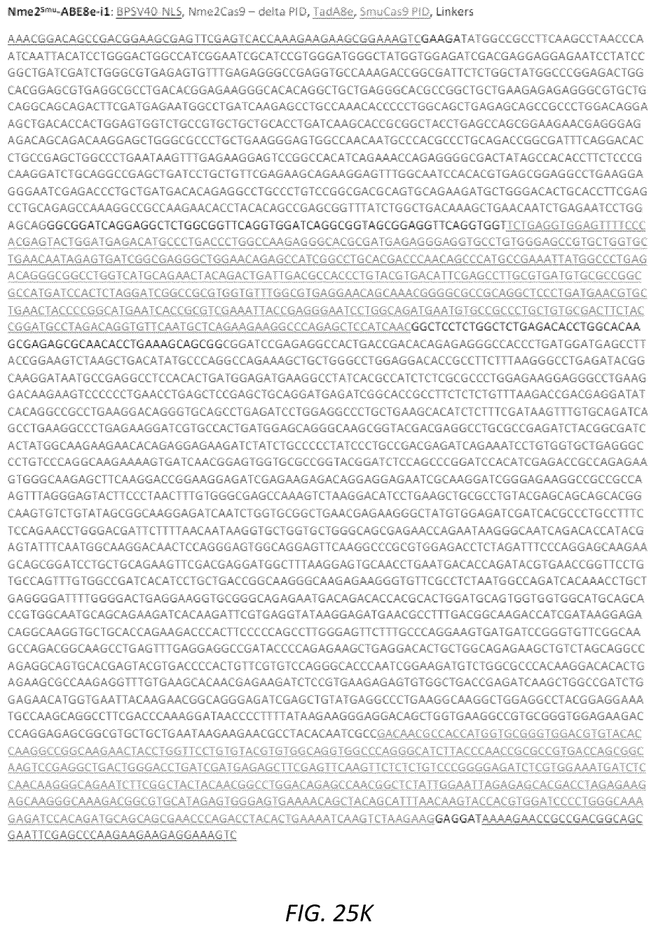

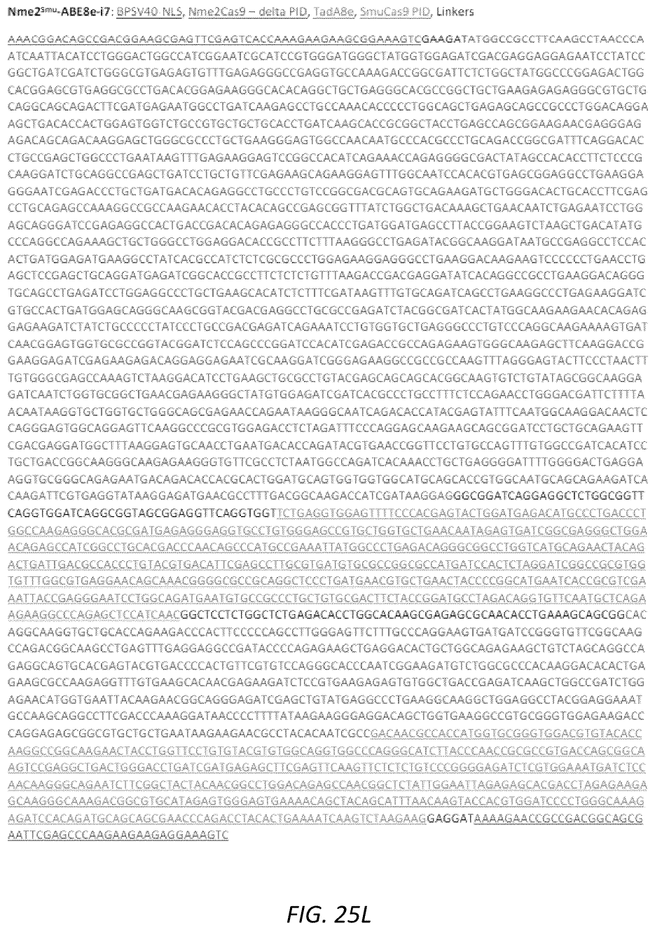

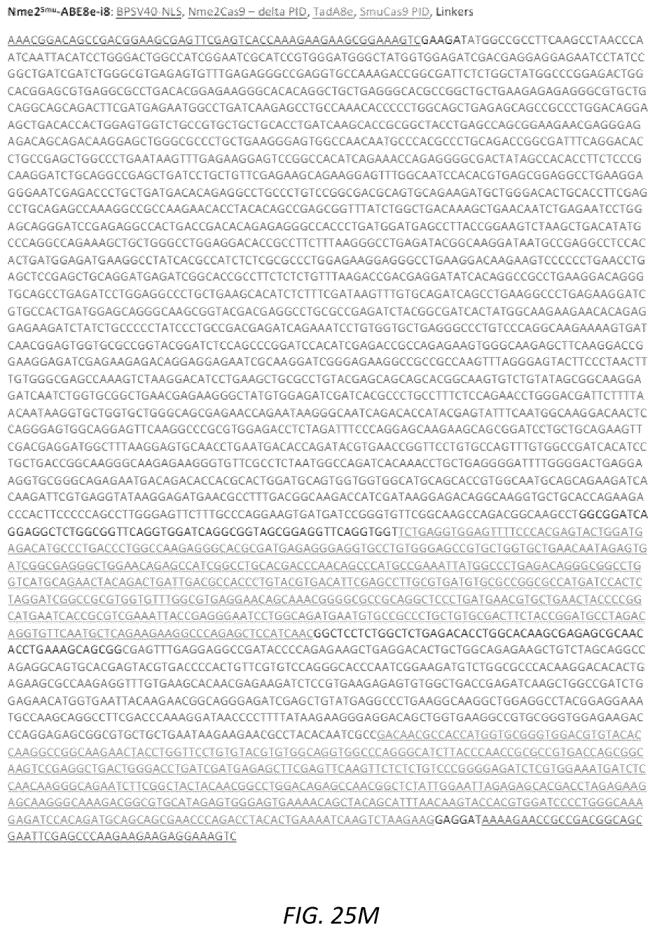

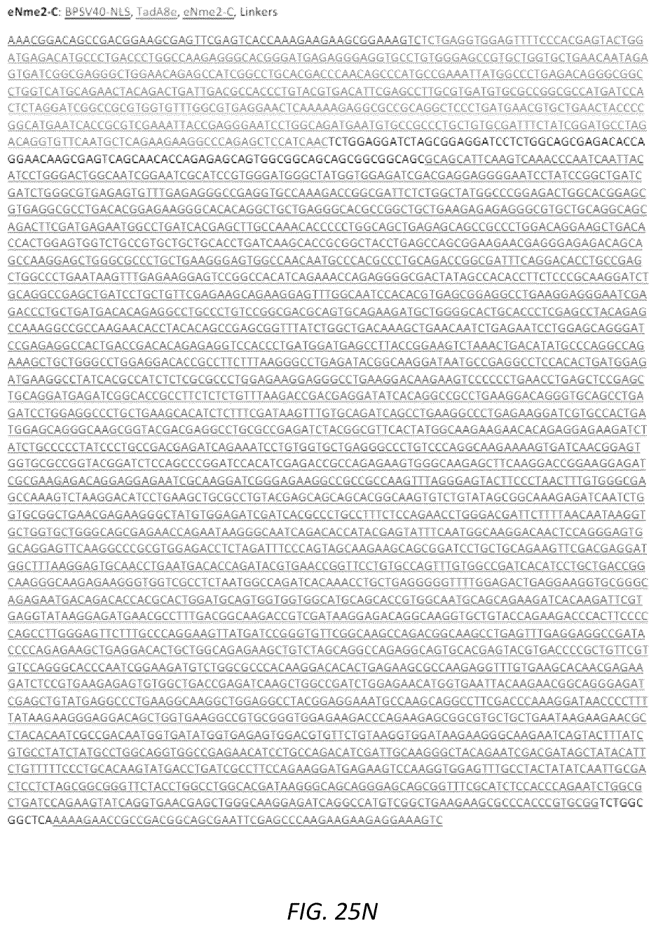

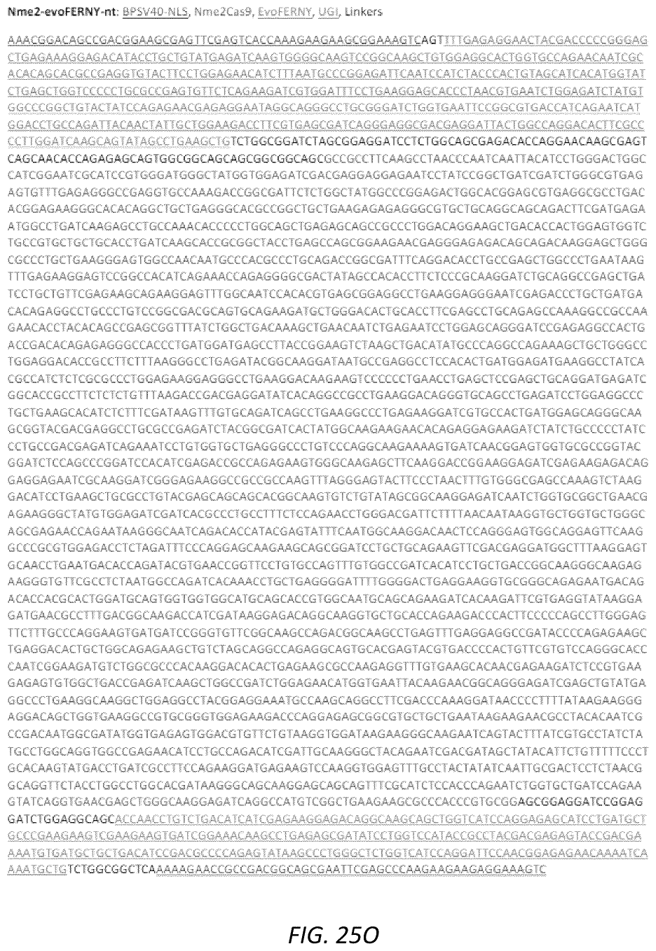

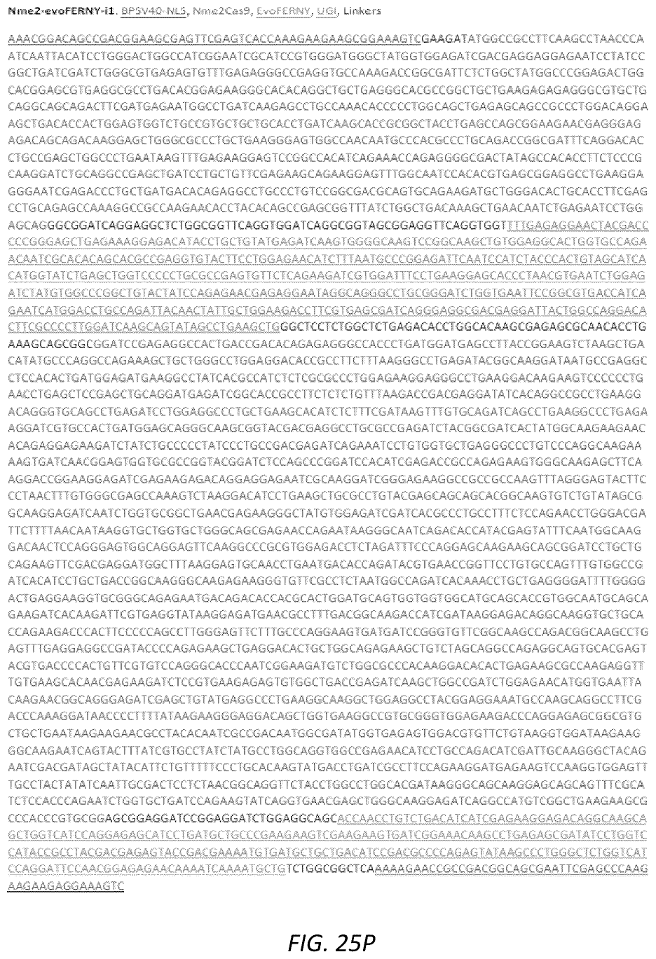

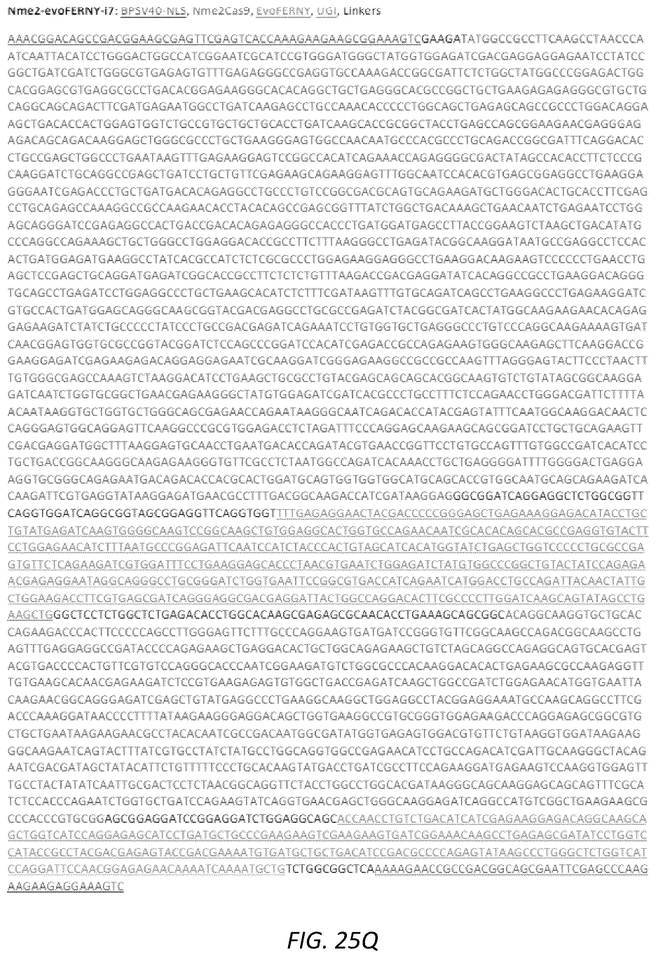

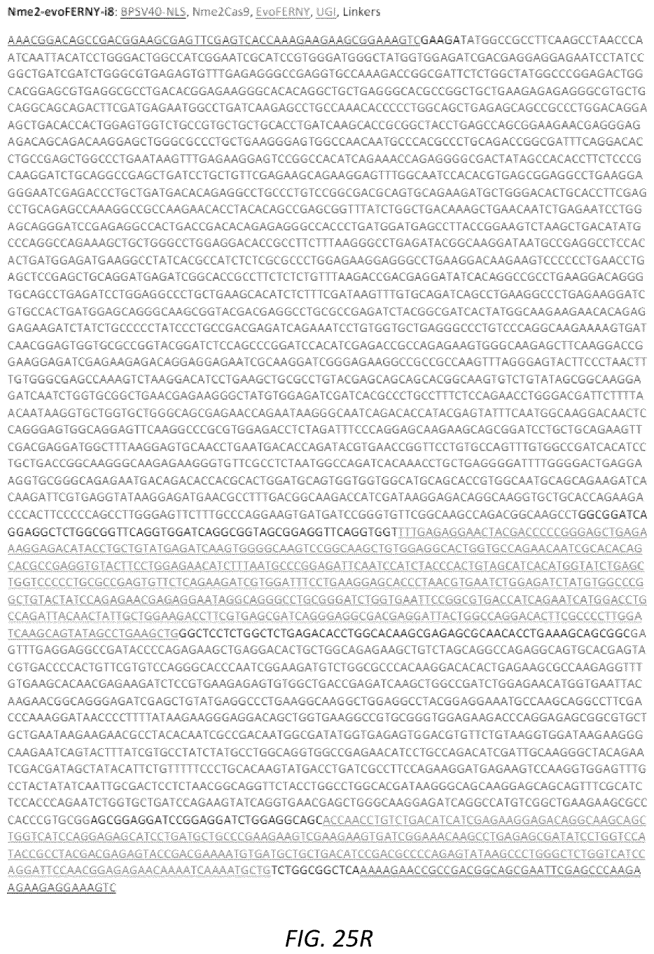

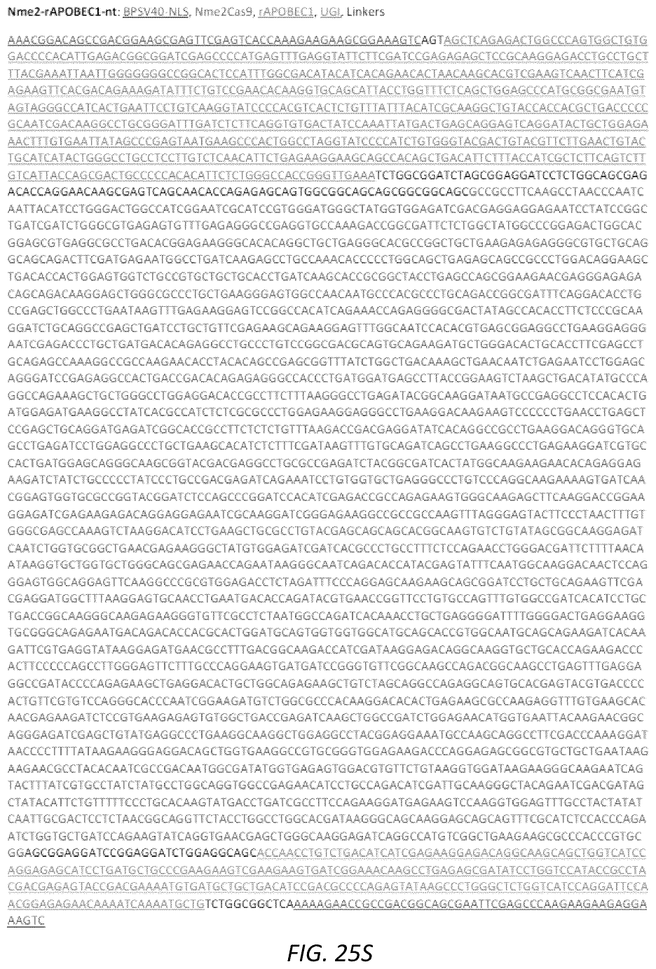

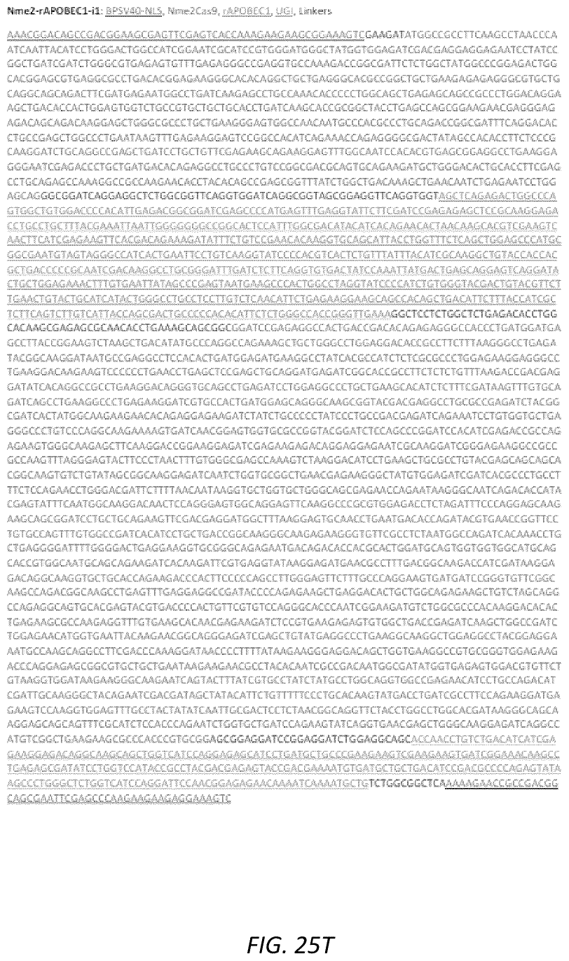

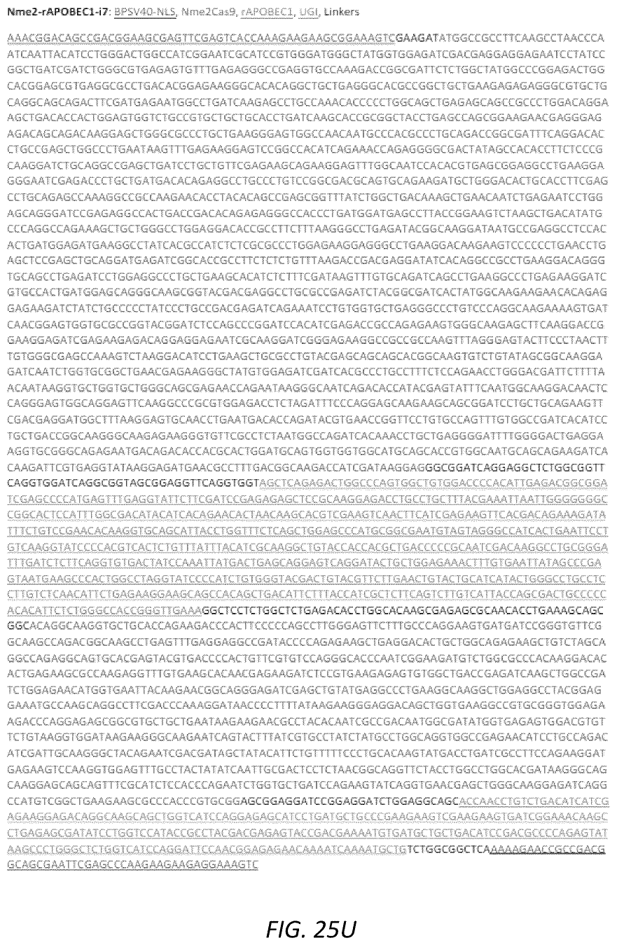

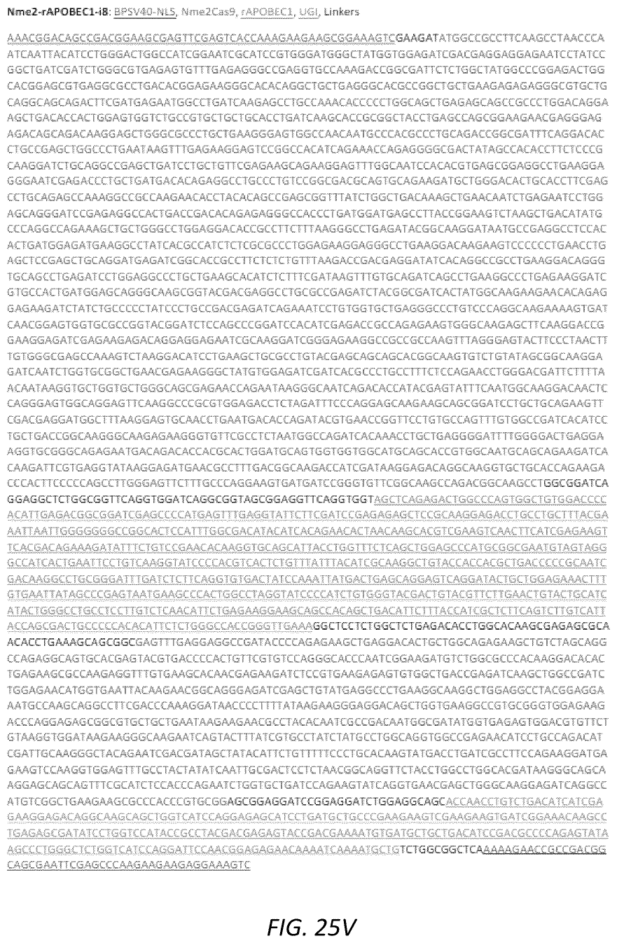

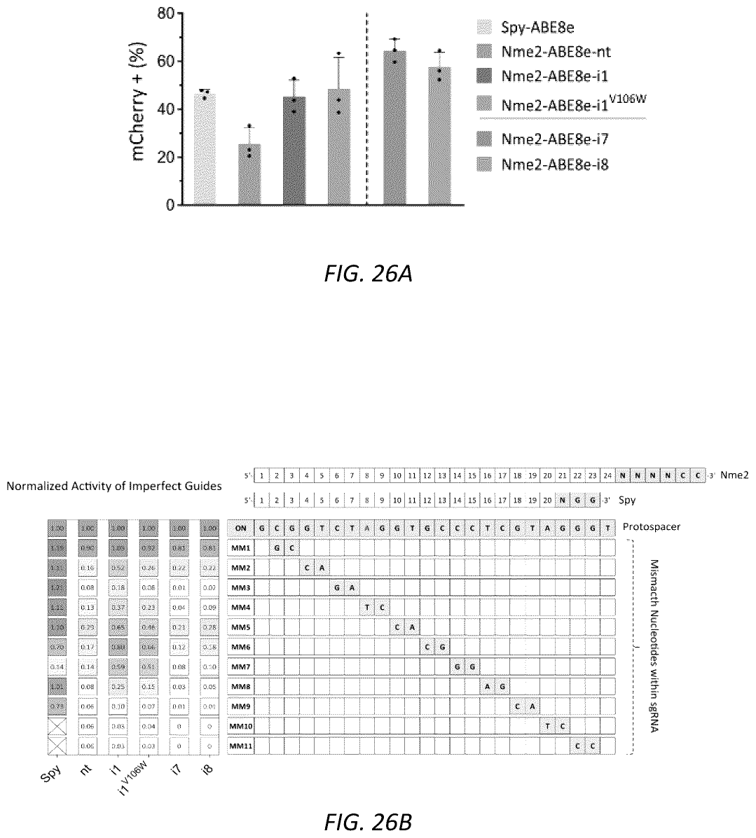

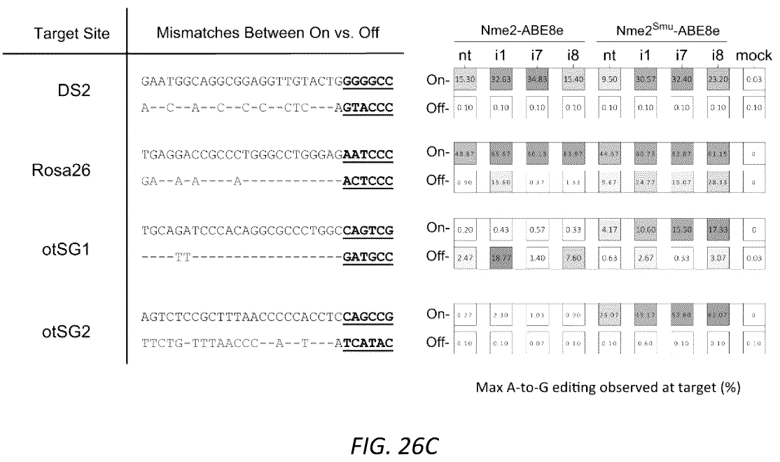

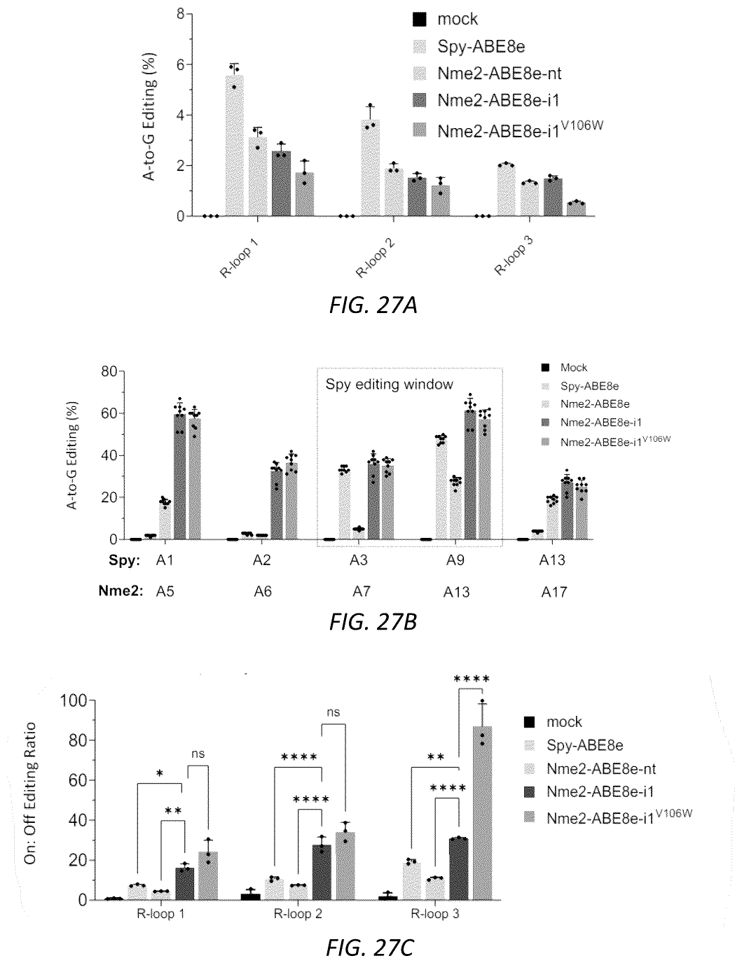

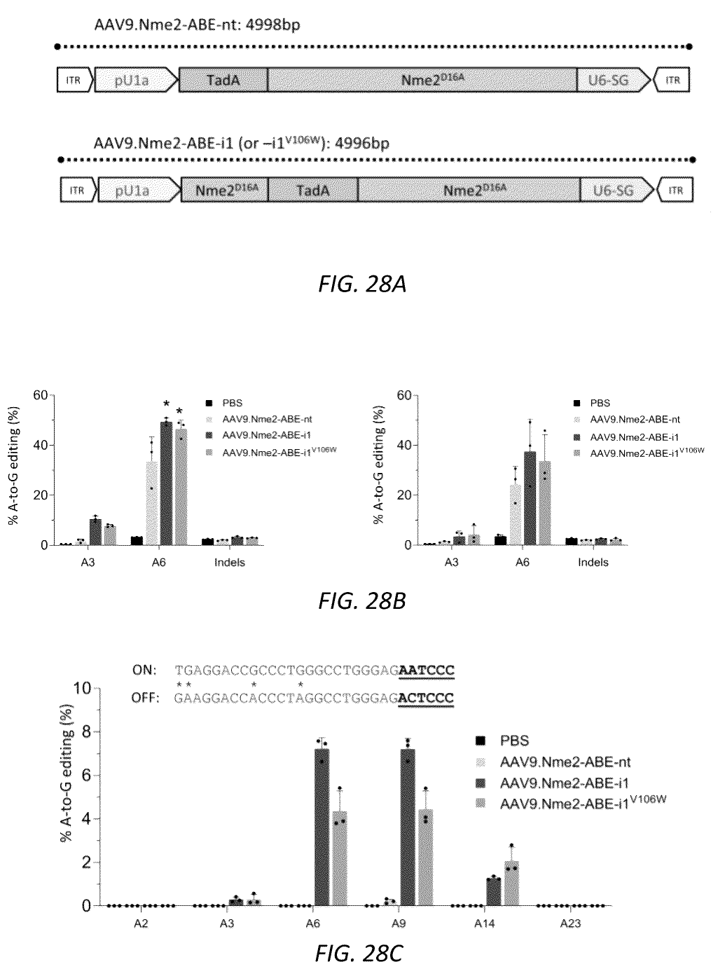

FIG. 25J: (SEQ ID NO:80) Displays the nucleotide sequence of Nme2^{Smu}-ABE8e-nt: BPSV40-NLS, Nme2Cas9—delta PID, TadA8e, SmuCas9 PID, Linkers.

FIG. 25K: (SEQ ID NO:81) Displays the nucleotide sequence of Nme2^{Smu}-ABE8e-i1: BPSV40-NLS, Nme2Cas9—delta PID, TadA8e, SmuCas9 PID, Linkers.

FIG. 25L: (SEQ ID NO:82) Displays the nucleotide sequence of Nme2^{Smu}-ABE8e-i7: BPSV40-NLS, Nme2Cas9—delta PID, TadA8e, SmuCas9 PID, Linkers.

FIG. 25M: (SEQ ID NO:83) Displays the nucleotide sequence of Nme2^{Smu}-ABE8e-i8: BPSV40-NLS, Nme2Cas9—delta PID, TadA8e, SmuCas9 PID, Linkers.

FIG. 25N: (SEQ ID NO:84) Displays the nucleotide sequence of eNme2-C: BPSV40-NLS, TadA8e, eNme2-C, Linkers.

FIG. 250: (SEQ ID NO:85) Displays the nucleotide sequence of Nme2-evoFERNY-nt: BPSV40-NLS, Nme2Cas9, EvoFERNY, UGI, Linkers.

FIG. 25P: (SEQ ID NO:86) Displays the nucleotide sequence of Nme2-evoFERNY-i1: BPSV40-NLS, Nme2Cas9, EvoFERNY, UGI, Linkers.

FIG. 25Q: (SEQ ID NO:87) Displays the nucleotide sequence of Nme2-evoFERNY-i7: BPSV40-NLS, Nme2Cas9, EvoFERNY, UGI, Linkers.

FIG. 25R: (SEQ ID NO:88) Displays the nucleotide sequence of Nme2-evoFERNY-i8: BPSV40-NLS, Nme2Cas9, EvoFERNY, UGI, Linkers.

FIG. 25S: (SEQ ID NO:89) Displays the nucleotide sequence of Nme2-rAPOBEC1-nt: BPSV40-NLS, Nme2Cas9, rAPOBEC1, UGI, Linkers.

FIG. 25T: (SEQ ID NO:90) Displays the nucleotide sequence of Nme2-rAPOBEC1-i1: BPSV40-NLS, Nme2Cas9, rAPOBEC1, UGI, Linkers.

FIG. 25U: (SEQ ID NO:91) Displays the nucleotide sequence of Nme2-rAPOBEC1-i7: BPSV40-NLS, Nme2Cas9, rAPOBEC1, UGI, Linkers.

FIG. 25V: (SEQ ID NO:92) Displays the nucleotide sequence of Nme2-rAPOBEC1-i8: BPSV40-NLS, Nme2Cas9, rAPOBEC1, UGI, Linkers.

FIGS. 26A-26C present specificities of domain-inlaid Nme2Cas9-ABE8e variants.

FIG. 26A: Displays a comparison of on-target activity of transfected Spy-ABE8e and Nme2-ABE8e effectors in activating the ABE mCherry reporter, as measured by flow cytometry (n=3 biological replicates; data represent mean±SD).

FIG. 26B: (SEQ ID NO(s):104-115) Displays the mismatch tolerance of Spy- or Nme2—ABE8e variants in ABE mCherry reporter cells at an overlapping target site positioning the target adenine for reporter activation at A8. Activities with single-guide RNAs carrying mismatched nucleotides as indicated (MM #, orange) are normalized to those of the fully complementary guides (ON, gray) (n=3 biological replicates) for each effector, as indicated in the columns to the left. Heatmap data by column represent the normalized mismatched tolerance of the tested effectors.

FIG. 26C: (SEQ ID NO(s):93-100) Displays comparison of Nme2-ABE8e variants at previously validated genomic targets. A-to-G editing was measured following transfection with WT or chimeric, PID-swapped Nme2-ABE8e plasmids at endogenous HEK293T or mouse N2A genomic loci following transfection. The editing efficiencies at the maximally edited adenine for the On- or Off-target site for each effector were marked in the heatmaps. Off-target mismatches to the spacer are denoted with red nucleotides, whereas dashes correspond to a matched nucleotide. Editing activities were measured by amplicon sequencing (n=3 biological replicates; data represent mean).

FIGS. 27A-27C present the specificity of Domain-Inlaid Nme2Cas9-ABE8e.

FIG. 27A: Displays guide-independent DNA off-target A-to-G editing at orthogonal SauCas9R-loops measured via amplicon deep sequencing. SauCas9 HNH nickase was used to increase the sensitivity of editing at the orthogonal R-loops (n=3 biological replicates; data represent mean±SD).

FIG. 27B: Displays on-target activity of the ABE8e variants tested for the R-loop assay with a PAMB:matched target site for Spy-ABE8e and Nme2-ABE8e effectors, measured via amplicon deep sequencing. SpyABE8e editing window is boxed. Overlapping target site sequence from 5′ to 3′ with adenines in red, and Spyand Nme2—PAMs bold and underlined (n=3 biological replicates per off-target R-loop in (c); data represent mean±SD).

FIG. 27C: Displays ratios of on-target vs. off-target editing of the ABE effectors tested at the overlapping Linc01588 target site and the orthogonal dSauCas9R-loops (n=3 biological replicates, data represent mean±SD). Two-way ANOVA analysis: ns, p>0.05; *, p<0.05; **, p<0.01; ***, p<0.001; ****, p<0.0001. On-target editing efficiency for Spy-ABE8e is derived from the mean editing within its editing window, so as not to skew the ratio when compared to the wider on-target editing window of Nme2-ABE8e.

FIGS. 28A-28C present in vivo editing with AAV9.Nme2-ABE8e-nt vs.−i1 vs. −i1^{V106W}.

FIG. 28A: Shows a schematic of the AAV constructs for the Nme2-ABE8e effectors.

FIG. 28B: Displays editing with AAV Nme2-ABE vectors in mouse liver (left) and striatum (right). Left, quantification of the editing efficiency at the Rosa26 locus by amplicon deep sequencing using liver genomic DNA from mice that were tail-vein-injected with the indicated vector at 4×10¹¹ vg/mouse (n=3 mice per group; data represent mean±SD). Nme2-ABE8e-i1 (p=0.04), Nme2-ABE-i1^{V106W} (p=0.015). Right, quantification of the editing efficiency at the Rosa26 locus by amplicon deep sequencing using striatum genomic DNA from mice intrastriatally injected with the indicated vector at 1×10¹⁰ vg/side (n=3 mice per group; data represent mean±SD). One-way ANOVA analysis: ns, p>0.05; *, p≤0.05.

FIG. 28C: (SEQ ID NO:95), (SEQ ID NO:101) Displays protospacer of the Rosa26 on-target site (“ON”) and a previously validated Nme2-ABE8e off-target site (OT1, “OFF”). Adenines are in red, mismatches in OT1 have asterisks, and PAM regions are bold and underlined. The bar graph shows quantification of A-to-G edits in amplicon deep sequencing reads at the OT1 site using liver genomic DNA from mice tail-vein injected in (FIG. 28B), with vectors indicated in the inset (n=3 mice per group; data represent mean±SD).

### DETAILED DESCRIPTION OF THE INVENTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target. Such a specific genomic target includes, but is not limited to, a chromosomal region, a gene, a promoter, an open reading frame or any nucleic acid sequence.

As used herein, the term “single base” refers to one, and only one, nucleotide within a nucleic acid sequence. When used in the context of single base editing, it is meant that the base at a specific position within the nucleic acid sequence is replaced with a different base. This replacement may occur by many mechanisms, including but not limited to, substitution or modification.

As used herein, the term “target” or “target site” refers to a pre-identified nucleic acid sequence of any composition and/or length. Such target sites include, but is not limited to, a chromosomal region, a gene, a promoter, an open reading frame or any nucleic acid sequence. In some embodiments, the present invention interrogates these specific genomic target sequences with complementary sequences of gRNA.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “viral vector” encompasses any nucleic acid construct derived from a virus genome capable of incorporating heterologous nucleic acid sequences for expression in a host organism. For example, such viral vectors may include, but are not limited to, adeno-associated viral vectors, lentiviral vectors, SV40 viral vectors, retroviral vectors, adenoviral vectors. Although viral vectors are occasionally created from pathogenic viruses, they may be modified in such a way as to minimize their overall health risk. This usually involves the deletion of a part of the viral genome involved with viral replication. Such a virus can efficiently infect cells but, once the infection has taken place, the virus may require a helper virus to provide the missing proteins for production of new virions. Preferably, viral vectors should have a minimal effect on the physiology of the cell it infects and exhibit genetically stable properties (e.g., do not undergo spontaneous genome rearrangement). Most viral vectors are engineered to infect as wide a range of cell types as possible. Even so, a viral receptor can be modified to target the virus to a specific kind of cell. Viruses modified in this manner are said to be pseudotyped. Viral vectors are often engineered to incorporate certain genes that help identify which cells took up the viral genes. These genes are called marker genes. For example, a common marker gene confers antibiotic resistance to a certain antibiotic.

As used herein, the term “genetic disease” refers to any medical condition having a primary causative factor of a mutated gene. The gene mutation may comprise a nucleic acid sequence wherein at least one, if not more, nucleotides are not wild type.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer DNA”. The spacers are short segments of DNA from a virus and may serve as a ‘memory’ of past exposures to facilitate an adaptive defense against future invasions.

As used herein, the term “Cas” or “CRISPR-associated (cas)” refers to genes often associated with CRISPR repeat-spacer arrays.

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence.

As used herein, the term “N-terminal domain” refers to the fusion of a first peptide or protein at the N-terminal end of a second peptide or protein. For example, a nucleotide deaminase protein may be “N-terminally” fused to the last amino acid of a Cas9 nuclease protein.

As used herein, the term “inlaid domain” refers to the fusion of a first protein between the N-terminal and C-terminal ends of a second protein. For example, a nucleotide deaminase protein is an “inlaid domain” when inserted between the N-terminal and C-terminal ends of a Cas9 nuclease protein.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “fluorescent protein” refers to a protein domain that comprises at least one organic compound moiety that emits fluorescent light in response to the appropriate wavelengths. For example, fluorescent proteins may emit red, blue and/or green light. Such proteins are readily commercially available including, but not limited to: i) mCherry (Clonetech Laboratories): excitation: 556/20 nm (wavelength/bandwidth); emission: 630/91 nm; ii) sfGFP (Invitrogen): excitation: 470/28 nm; emission: 512/23 nm; iii) TagBFP (Evrogen): excitation 387/11 nm; emission 464/23 nm.

As used herein, the term “sgRNA” refers to single guide RNA used in conjunction with CRISPR associated systems (Cas). sgRNAs contains nucleotides of sequence complementary to the desired target site. Watson-crick pairing of the sgRNA with the target site recruits the nuclease-deficient Cas9 to bind the DNA at that locus.

As used herein, the term “orthogonal” refers targets that are non-overlapping, uncorrelated, or independent. For example, if two orthogonal nuclease-deficient Cas9 gene fused to different effector domains were implemented, the sgRNAs coded for each would not cross-talk or overlap. Not all nuclease-deficient Cas9 genes operate the same, which enables the use of orthogonal nuclease-deficient Cas9 gene fused to a different effector domains provided the appropriate orthogonal sgRNAs.

As used herein, the term “phenotypic change” or “phenotype” refers to the composite of an organism's observable characteristics or traits, such as its morphology, development, biochemical or physiological properties, phenology, behavior, and products of behavior. Phenotypes result from the expression of an organism's genes as well as the influence of environmental factors and the interactions between the two.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed to a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/L NaCl, 6.9 g/L NaH₂PO₄ H₂O and 1.85 g/L EDTA, pH adjusted to 7.4 with NaOH), 0.1% SDS, 5×Denhardt's reagent {50×Denhardt's contains per 500 mL: 5 g Ficoll (Type 400, Pharmacia), 5 g BSA (Fraction V; Sigma)} and 100 g/mL denatured salmon sperm DNA followed by washing in a solution comprising 5×SSPE, 0.1% SDS at 42° C. when a probe of about 500 nucleotides in length is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

### I. CRISPR Cas9 Gene Editors

N. meningitidis Cas9 RNA-Guided Nucleases

N. meningitidis RNA-guided nucleases (e.g., Nme Cas9 or NmCas9) according to the present disclosure include, without limitation, any Cas9 nuclease obtained from N. meningitidis (e.g., Nme1Cas9, Nme2Cas9, or Nme3Cas9), as well as other Cas9 nucleases derived or obtained therefrom. N. meningitidis Cas9 nucleases belong to the Type II-C Cas9 nucleases, which are generally less than 1,100 amino acids in length and are capable of genome editing, including genome editing in mammalian cells. In functional terms, N. meningitidis RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Nme1Cas9, Nme2Cas9, or Nme3Cas9), or variation (e.g., full-length vs. truncated or split; naturally-occurring PAM specificity vs. engineered PAM specificity). The PAM sequence recognized by the Nme2Cas9 nucleases of the disclosure include N₄CC (see, Sun et al., supra; Edraki et al., supra).

Nme Cas9 nucleases are described in further detail in Esvelt et al. (Nat. Methods. 10: 1116-1121. 2013); Hou et al. (PNAS. 110: 15644-15649. 2013); Lee et al. (Mol. Thera. 24: 645-654. 2016); Amrani et al. (Genome Biol. 19: 214. 2018); Edraki et al. (Mol. Cell. 73: 714-726. 2019); U.S. Patent Publication 2014/0349405; U.S. Pat. No. 10,190,106; U.S. Patent Publication 2018/0355331; and U.S. Patent Publication 2019/0338308, each of which is incorporated herein by reference.

### Nme2Cas9 PAM Interacting Domains

Protospacer adjacent motif (PAM) recognition by Cas9 orthologs occurs predominantly through protein-DNA interactions between the PAM Interacting Domain (PID) and the nucleotides adjacent to the protospacer (Jiang and Doudna, 2017). PAM mutations often enable phage escape from type II CRISPR immunity (Paez-Espino et al., 2015), placing these systems under selective pressure not only to acquire new CRISPR spacers, but also to evolve new PAM specificities via PID mutations. In addition, some phages and MGEs express anti-CRISPR (Acr) proteins that inhibit Cas9 (Pawluk et al., 2016; Hynes et al., 2017; Rauch et al., 2017). PID binding is an effective inhibitory mechanism adopted by some Acrs (Dong et al., 2017; Shin et al., 2017; Yang and Patel, 2017), suggesting that PID variation may also be driven by selective pressure to escape Acr inhibition. Cas9 PIDs can evolve such that closely-related orthologs recognize distinct PAMs, as illustrated recently in two species of Geobacillus. The Cas9 encoded by G. stearothermophilus recognizes a N₄CRAA PAM, but when its PID was swapped with that of strain LC300's Cas9, its PAM requirement changed to N₄GMAA (Harrington et al., 2017b).

In one embodiment, the present disclosure contemplates a chimeric Nme2Cas9 protein in which the Nme2Cas9 PID is replaced with the PID of Simonsiella muelleri Cas9 (SmuCas9). This chimeric Nme2Cas9 is designated Nme2^{Smu}Cas9 herein. The PAM recognized by Nme2^{Smu}Cas9 is expanded beyond N₄CC (the WT Nme2Cas9 PAM), to N₄CN (e.g., N₄CC, N₄CT, N₄CG, and N₄CA), thereby greatly expanding the number of potential target sites in the genome. Exemplary Nme2Cas9 and Nme2^{Smu}Cas9 amino acid sequences are provided herein in Table 1. Nme2^{Smu}Cas9 is described in further detail in PCT/US22/48261, incorporated herein by reference.

In certain embodiments, the Nme2Cas9 (e.g., the Nme2Cas9 variant) comprises a PID that interacts with an N₄CC nucleotide sequence, an N₄CA nucleotide sequence, an N₄CG nucleotide sequence, an N₄CT nucleotide sequence, or an N₄C nucleotide sequence.

In certain embodiments, the PID is an Nme2Cas9 PID or an SmuCas9 PID.

In certain embodiments, the Nme2Cas9 PID comprises an amino acid sequence set forth in (DNGDMVRVDVFCKVDKKGKNQYFIVPIYAWQVAENILPDIDCKGYRIDDSYTFCFSL HKYDLIAFQKDEKSKVEFAYYINCDSSNGRFYLAWHDKGSKEQQFRISTQNLVLIQKY QVNELGKEIRPCRLKKRPPVR) (SEQ ID NO:27).

In certain embodiments, the SmuCas9 PID comprises an amino acid sequence set forth in (DNATMVRVDVYTKAGKNYLVPVYVWQVAQGILPNRAVTSGKSEADWDLIDESFEFK FSLSRGDLVEMISNKGRIFGYYNGLDRANGSIGIREHDLEKSKGKDGVHRVGVKTATA FNKYHVDPLGKEIHRCSSEPRPTLKIKSKK) (SEQ ID NO:28).

### Nme2Cas9 Variants

Described herein are Nme2Cas9 and Nme2^{Smu}Cas9 variants with increased genome editing activities (e.g., nuclease and base editing efficiencies) in mammalian cells. Specific amino acid substitutions were selected by rational design and screening that increased editing activities of Nme2Cas9 and Nme2^{Smu}Cas9 for both nuclease editing and base editing.

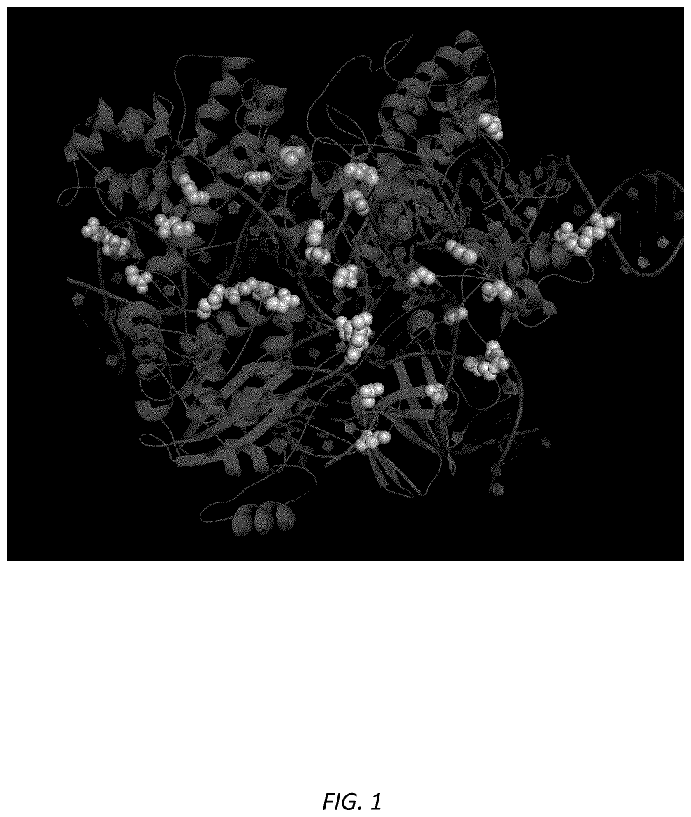

In certain embodiments, one or more amino acid substitutions are introduced into amino acids that contact the target strand (TS) DNA. In certain embodiments, one or more amino acid substitutions are introduced into amino acids that contact the non-target strand (NTS) DNA. In certain embodiments, one or more amino acid substitutions are introduced into amino acids that contact the sgRNA (SG). This is exemplified in FIG. 1.

In one aspect, the disclosure provides a Neisseria meningitidis (Nme) 2 Cas9 (Nme2Cas9) variant comprising an amino acid substitution at one or more positions selected from the group consisting of E520, D873, D418, E471, D442, E844, E443, D470, E585, E552, D451, E587, E508, E932, D56, D1048, E1079, D660, E887, T72, and E186. In certain embodiments, the target strand contacting positions correspond to E520, D873, D418, E471, D442, E844, E443, D470, E585, E552, D451, E587, and E508. In certain embodiments, the non-target strand and sgRNA contacting positions correspond to E932, D56, D1048, E1079, D660, E887, T72, and E186. The recited amino acid positions are relative to an amino acid sequence of SEQ ID NO: 1 (WT Nme2Cas9) or SEQ ID NO: 2 (Nme2^{Smu}Cas9). All of the recited amino acid positions are present in both Nme2Cas9 and Nme2^{Smu}Cas9, with the exception of positions D1048 and E1079, which are only present in the Smu PID of Nme2^{Smu}Cas9.

In certain embodiments, the Nme2Cas9 or Nme2^{Smu}Cas9 comprises 1, 2, 3, 4, or 5 amino acid substitutions (i.e., 1, 2, 3, 4, or 5 amino acid substitutions from the amino acid positions of E520, D873, D418, E471, D442, E844, E443, D470, E585, E552, D451, E587, E508, E932, D56, D1048, E1079, D660, E887, T72, and E186).

In certain embodiments, the Nme2Cas9 or Nme2^{Smu}Cas9 comprises or consists of amino acid substitutions at positions E932 and D873; E932 and D56; E932 and E520; E932 and D1048; D873 and D56; D873 and E520; D873 and D1048; D56 and E520; D56 and D1048; E520 and D1048; E932, D873, and D56; E932, D873, and E520; E932, D873, and D1048; E932, D56, and E520; E932, D56, and D1048; E932, E520, and D1048; D873, D56, and E520; D873, D56, and D1048; D873, E520, and D1048; D56, E520, and D1048; E932, D873, D56, and E520; E932, D873, D56, and D1048; E932, D56, E520, and D1048; D873, D56, E520, and D1048; or E932, D873, D56, E520, and D1048.

In certain embodiments, the amino acid substitution is a positively charged amino acid. In certain embodiments, the amino acid substitution is an arginine (R), lysine (K), or histidine (H). In certain embodiments, the amino acid substitution is an arginine (R). In certain embodiments, the Nme2Cas9 or Nme2^{Smu}Cas9 comprises an amino acid substitution of any one or more of E520R, D873R, D418R, E471R, D442R, E844R, E443R, D470R, E585R, E552R, D451R, E587R, E508R, E932R, D56R, D1048R, E1079R, D660R, E887R, T72R, and E186R.

In certain embodiments, the Nme2Cas9 or Nme2^{Smu}Cas9 comprises an amino acid substitution of any one or more of E520R, D873R, D418R, E471R, D442R, E844R, E443R, E932R, D56R, D1048R, E1079R, D660R, E887R, T72R, and E186R.

In certain embodiments, the Nme2Cas9 or Nme2^{Smu}Cas9 comprises amino acid substitutions E932R and D873R; E932R and D56R; E932R and E520R; E932R and D1048R; D873R and D56R; D873R and E520R; D873R and D1048R; D56R and E520R; D56R and D1048R; E520R and D1048R; E932R, D873R, and D56R; E932R, D873R, and E520R; E932R, D873R, and D1048R; E932R, D56R, and E520R; E932R, D56R, and D1048R; E932R, E520R, and D1048R; D873R, D56R, and E520R; D873R, D56R, and D1048R; D873R, E520R, and D1048R; D56R, E520R, and D1048R; E932R, D873R, D56R, and E520R; E932R, D873R, D56R, and D1048R; E932R, D56R, E520R, and D1048R; D873R, D56R, E520R, and D1048R; or E932R, D873R, D56R, E520R, and D1048R. 3′

### Base Editor Fusion Proteins

The Nme2Cas9 and Nme2^{Smu}Cas9 variants described herein may serve as the Cas9 domain of a base editor fusion protein. Nucleotide base editors (NBEs), such as cytosine and adenine base editors (CBEs and ABEs) were developed as a way to precisely correct point mutations without inducing double-strand breaks or requiring a DNA donor. Base editor fusion proteins are comprised of a catalytically impaired Cas9 domain that is completely inactive or cleaves only one strand (a.k.a. dead/dCas9 or nickase/nCas9, respectively) fused to one or more cytosine deaminase (CBE) or adenine deaminase (ABE) domains. For efficient base editing to occur, the Cas9 base editor fusion must recognize a short sequence motif, called a PAM, adjacent to the target site, and a target adenine within an “editing window” upstream of PAM. The PAM and editing window are defined by the Cas domain, deaminase, and the type of fusion between the two effectors.

In certain embodiments, the Nme2Cas9 and Nme2^{Smu}Cas9 variants of the disclosure further comprises a nucleotide base editor (NBE) domain fused to the Nme2Cas9 variant or Nme2^{Smu}Cas9 variant.

### NBE Domains

In certain embodiments, the NBE domain (i.e., an inlaid NBE domain or terminal NBE domain) is an adenine base editor (ABE) domain. In certain embodiments, the ABE domain is an inlaid adenosine deaminase protein domain. In certain embodiments, the adenosine deaminase protein domain is an adenosine deaminase 8e protein domain (TadA8e). In certain embodiments, the TadA8e comprises an amino acid sequence set forth in SEQ ID NO: 9 (SEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNSKRGAA GSLMNVLNYPGMNHRVEITEGILADECAALLCDFYRMPRQVFNAQKKAQSSIN), or an amino acid sequence comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, Or 100% identity) to SEQ ID NO: 9.

In certain embodiments, the NBE domain (i.e., an inlaid NBE domain or terminal NBE domain) is a cytidine base editor (CBE) domain. In certain embodiments, the CBE domain is an inlaid cytosine deaminase protein domain. In certain embodiments, the cytosine deaminase protein domain is evoFERNY or rAPOBEC1. In certain embodiments, the evoFERNY comprises an amino acid sequence set forth in SEQ ID NO: 13 (FERNYDPRELRKETYLLYEIKWGKSGKLWRHWCQNNRTQHAEVYFLENIFNARRFNP STHCSITWYLSWSPCAECSQKIVDFLKEHPNVNLEIYVARLYYPENERNRQGLRDLVN SGVTIRIMDLPDYNYCWKTFVSDQGGDEDYWPGHFAPWIKQYSLKL) or an amino acid sequence comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, Or 100% identity) to SEQ ID NO: 13.

In certain embodiments, the rAPOBEC1 comprises an amino acid sequence set forth in SEQ ID NO: 11 (SSETGPVAVDPTLRRRIEPHEFEVFFDPRELRKETCLLYEINWGGRHSIWRHTSQNTNK HVEVNFIEKFTTERYFCPNTRCSITWFLSWSPCGECSRAITEFLSRYPHVTLFIYIARLYH HADPRNRQGLRDLISSGVTIQIMTEQESGYCWRNFVNYSPSNEAHWPRYPHLWVRLY VLELYCIILGLPPCLNILRRKQPQLTFFTIALQSCHYQRLPPHILWATGLK) or an amino acid sequence comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, Or 100% identity) to SEQ ID NO: 11.

Where the percent identity of any one of SEQ ID NO: 9, 11, and 13 is less than 100%, it will be understood that the NBE domain will retain the base editing activity described herein.

In certain embodiments, the Nme2Cas9 variant or Nme2^{Smu}Cas9 variant further comprises a uracil glycosylase inhibitor (UGI). A UGI may be expressed as a separate protein or also linked to the fusion protein comprising the Nme2Cas9 protein and NBE domain. The UGI is capable of enhancing the base editing activity of a CBE domain. The CBE domain mediates a C to T change by creating a U on the free DNA strand. This U may be transformed into an apurinic/apyrimidinic (AP) site by various DNA glycosylases. A UGI may prevent the transformation of the U into an AP.

### Inlaid NBE Domain Fusions

In certain embodiments, the NBE domain is an inlaid NBE domain inserted into the Nme2Cas9 variant or Nme2^{Smu}Cas9 variant.

In certain embodiments, the inlaid NBE domain is inserted into a REC domain of the Nme2Cas9 variant or Nme2^{Smu}Cas9 variant.

In certain embodiments, the inlaid NBE domain is inserted into a HNH domain of the Nme2Cas9 variant or Nme2^{Smu}Cas9 variant.

In certain embodiments, the inlaid NBE domain is inserted into a RuvC domain of the Nme2Cas9 variant or Nme2^{Smu}Cas9 variant.

In certain embodiments, the inlaid NBE domain is inserted between amino acid position 291 and amino acid position 292 of the Nme2Cas9 variant or Nme2^{Smu}Cas9 variant, relative to an amino acid sequence of SEQ ID NO: 1 or 2. Base editor fusion proteins with an inlaid NBE domain inserted between amino acid position 291 and amino acid position 292 are referred to herein as NBE-i1 base editors (such as ABE8e-i1).

In certain embodiments, the inlaid NBE domain is inserted between amino acid position 761 and amino acid position 762 of the Nme2Cas9 variant or Nme2^{Smu}Cas9 variant, relative to an amino acid sequence of SEQ ID NO: 1 or 2. Base editor fusion proteins with an inlaid NBE domain inserted between amino acid position 761 and amino acid position 762 are referred to herein as NBE-i7 base editors (such as ABE8e-i7).

In certain embodiments, the inlaid NBE domain is inserted between amino acid position 795 and amino acid position 796 of the Nme2Cas9 variant or Nme2^{Smu}Cas9 variant, relative to an amino acid sequence of SEQ ID NO: 1 or 2. Base editor fusion proteins with an inlaid NBE domain inserted between amino acid position 795 and amino acid position 796 are referred to herein as NBE-i8 base editors (such as ABE8e-i8).

The inlaid NBE domain may be flanked at the NBE domain N-terminus and/or NBE domain C-terminus by an amino acid linker. In other embodiments, the NBE domain may be directly linked (i.e., no amino acid linker) to the Nme2Cas9 variant or Nme2^{Smu}Cas9 variant at the inlaid position (i.e., between amino acid positions 291 and 292, 761 and 762, or 795 and 796).

In certain embodiments, the amino acid linker comprises a (GGS)ₙ(SEQ ID NO:41) linker, wherein n corresponds to 1-7. In certain embodiments, the amino acid linker comprises GGSGGSGGSGGSGGSGGSGG(SEQ ID NO: 15). In certain embodiments, the amino acid linker comprises GGS. In certain embodiments, the amino acid linker comprises GGSGGS(SEQ ID NO:36). In certain embodiments, the amino acid linker comprises GGSGGSGGS(SEQ ID NO:37). In certain embodiments, the amino acid linker comprises GGSGGSGGSGGS(SEQ ID NO:38). In certain embodiments, the amino acid linker comprises GGSGGSGGSGGSGGS(SEQ ID NO:39). In certain embodiments, the amino acid linker comprises SGGSGGSGGS(SEQ ID NO: 17). In certain embodiments, the amino acid linker comprises GGSGG(SEQ ID NO: 19).

In certain embodiments, the amino acid linker consists of the six hydrophilic, chemically stable amino acids A, E, G, P, S and T. In certain embodiments, the amino acid linker comprises GSSGSETPGTSESATPESSG(SEQ ID NO: 21). In certain embodiments, the amino acid linker comprises ETPGTSESAT(SEQ ID NO: 23). In certain embodiments, the amino acid linker comprises GTSES(SEQ ID NO: 25).

In certain embodiments, the amino acid linker comprises a sequence selected from the group consisting of: GGSGGSGGSGGSGGSGGSGG(SEQ ID NO: 15), SGGSGGSGGS(SEQ ID NO: 17), GGSGG(SEQ ID NO: 19), GSSGSETPGTSESATPESSG(SEQ ID NO: 21), ETPGTSESAT(SEQ ID NO: 23), and GTSES(SEQ ID NO: 25).

In certain embodiments, the amino acid linker comprises ED.

In certain embodiments, the inlaid NBE domain is flanked at the inlaid NBE domain N-terminus by an amino acid linker and the inlaid NBE domain C-terminus lacks an amino acid linker. In certain embodiments, the inlaid NBE domain is flanked at the inlaid NBE domain C-terminus by an amino acid linker and the inlaid NBE domain N-terminus lacks an amino acid linker. In certain embodiments, the amino acid linker at the inlaid NBE domain N-terminus is different than the amino acid linker at the inlaid NBE domain C-terminus. In certain embodiments, the amino acid linker at the inlaid NBE domain N-terminus is identical to the amino acid linker at the inlaid NBE domain C-terminus.

In certain embodiments, the inlaid NBE domain is flanked at the inlaid NBE domain N-terminus by GGSGGSGGSGGSGGSGGSGG(SEQ ID NO: 15) and at the inlaid NBE domain C-terminus by GSSGSETPGTSESATPESSG(SEQ ID NO: 21). In certain embodiments, the inlaid NBE domain is flanked at the inlaid NBE domain N-terminus by GSSGSETPGTSESATPESSG(SEQ ID NO: 21) and at the inlaid NBE domain C-terminus by GGSGGSGGSGGSGGSGGSGG(SEQ ID NO: 15).

### Terminal NBE Domain Fusions

In certain embodiments, the NBE domain is linked via an amino acid linker to the N-terminus of the Nme2Cas9 variant or Nme2^{Smu}Cas9 variant (i.e., not an inlaid NBE domain).

In certain embodiments, the NBE domain is linked via an amino acid linker to the C-terminus of the Nme2Cas9 variant or Nme2^{Smu}Cas9 variant (i.e., not an inlaid NBE domain).

In certain embodiments, the amino acid linker comprises a (GGS)ₙ(SEQ ID NO:41) linker, wherein n corresponds to 1-7. In certain embodiments, the amino acid linker comprises GGSGGSGGSGGSGGSGGSGG(SEQ ID NO: 15). In certain embodiments, the amino acid linker comprises GGS. In certain embodiments, the amino acid linker comprises GGSGGS(SEQ ID NO:36). In certain embodiments, the amino acid linker comprises GGSGGSGGS(SEQ ID NO:37). In certain embodiments, the amino acid linker comprises GGSGGSGGSGGS(SEQ ID NO:38). In certain embodiments, the amino acid linker comprises GGSGGSGGSGGSGGS(SEQ ID NO:39). In certain embodiments, the amino acid linker comprises SGGSGGSGGS(SEQ ID NO: 17). In certain embodiments, the amino acid linker comprises GGSGG(SEQ ID NO: 19).

In certain embodiments, the amino acid linker consists of the six hydrophilic, chemically stable amino acids A, E, G, P, S and T. In certain embodiments, the amino acid linker comprises GSSGSETPGTSESATPESSG(SEQ ID NO: 21). In certain embodiments, the amino acid linker comprises ETPGTSESAT(SEQ ID NO: 23). In certain embodiments, the amino acid linker comprises GTSES(SEQ ID NO: 25).

In certain embodiments, the amino acid linker comprises a sequence selected from the group consisting of: GGSGGSGGSGGSGGSGGSGG(SEQ ID NO: 15), SGGSGGSGGS(SEQ ID NO: 17), GGSGG(SEQ ID NO: 19), GSSGSETPGTSESATPESSG(SEQ ID NO: 21), ETPGTSESAT(SEQ ID NO: 23), and GTSES(SEQ ID NO: 25).

In certain embodiments, the amino acid linker comprises ED.

### Inlaid Base Editor Fusion Protein Linker Optimization

Adeno-associated viruses (AAVs) are useful viral vectors for the delivery of therapeutic proteins to subjects. However, the packaging size limit of an AAV is 4.8 kb to 5.0 kb, which includes the 5′ ITR and 3′ ITR sequences, the promoter sequence, and terminator sequence. The closer the AAV vector size is to 5.0 kb, the worse AAV packaging becomes. By way of example, an AAV9 Nme2Cas9-ABE-i1 has a vector size of−4.9 kb, right against the packaging limit, with the Nme2Cas9-ABE-i1 transgene contributing 3987 bp to the vector size. The Nme2^{Smu}Cas9-ABE-i1 is 4011 bp, 24 bp larger (8 amino acids) than the Nme2Cas9-ABE−i1. Accordingly, there exists a need to reduce the transgene size of the inlaid base editors described herein to improve AAV compatibility without sacrificing base editor activity. To achieve this result, the instant disclosure describes the optimization of amino acid linker length between the inlaid NBE domain and the Nme2Cas9.

In one aspect, the disclosure provides a fusion protein comprising a Neisseria meningitidis (Nme) 2 Cas9 (Nme2Cas9) protein and an inlaid nucleotide base editor (NBE) domain, wherein the inlaid NBE domain is flanked at the inlaid NBE domain N-terminus and/or C-terminus by an amino acid linker, or a linker is absent, and wherein the total number of amino acid linker residues is less than 40 amino acids.

Non-limiting examples of the Nme2Cas9 protein include a WT Nme2Cas9 protein, a chimeric Nme2^{Smu}Cas9 protein described herein, an Nme2Cas9 variant described herein, or a Nme2^{Smu}Cas9 variant described herein.

In certain embodiments, the amino acid linker that is present at the N-terminus of the inlaid NBE domain is 20 amino acids, and the amino acid linker that is present at the C-terminus of the inlaid NBE domain is 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, 1 amino acid, or is absent.

In certain embodiments, the amino acid linker that is present at the N-terminus of the inlaid NBE domain is 20 amino acids, and the amino acid linker that is present at the C-terminus of the inlaid NBE domain is 10 amino acids, 5 amino acids, or is absent.

In certain embodiments, the amino acid linker that is present at the C-terminus of the inlaid NBE domain is 20 amino acids, and the amino acid linker that is present at the N-terminus of the inlaid NBE domain is 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, 1 amino acid, or is absent.

In certain embodiments, the amino acid linker that is present at the C-terminus of the inlaid NBE domain is 20 amino acids, and the amino acid linker that is present at the N-terminus of the inlaid NBE domain is 10 amino acids, 5 amino acids, or is absent.

In certain embodiments, the amino acid linker that is present at the N-terminus of the inlaid NBE domain is 10 amino acids, and the amino acid linker that is present at the C-terminus of the inlaid NBE domain is 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, 1 amino acid, or is absent.

In certain embodiments, the amino acid linker that is present at the N-terminus of the inlaid NBE domain is 10 amino acids, and the amino acid linker that is present at the C-terminus of the inlaid NBE domain is 20 amino acids, 10 amino acids, 5 amino acids, or is absent.

In certain embodiments, the amino acid linker that is present at the N-terminus of the inlaid NBE domain is 5 amino acids, and the amino acid linker that is present at the C-terminus of the inlaid NBE domain is 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, 1 amino acid, or is absent.

In certain embodiments, the amino acid linker that is present at the N-terminus of the inlaid NBE domain is 5 amino acids, and the amino acid linker that is present at the C-terminus of the inlaid NBE domain is 20 amino acids, 10 amino acids, 5 amino acids, or is absent.

In certain embodiments, the amino acid linker that is present at the N-terminus of the inlaid NBE domain is absent, and the amino acid linker that is present at the C-terminus of the inlaid NBE domain is 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, 1 amino acid, or is absent.

In certain embodiments, the amino acid linker that is present at the N-terminus of the inlaid NBE domain is absent, and the amino acid linker that is present at the C-terminus of the inlaid NBE domain is 20 amino acids, 10 amino acids, 5 amino acids, or is absent.

In certain embodiments, the amino acid linker comprises a sequence selected from the group consisting of: GGSGGSGGSGGSGGSGGSGG(SEQ ID NO: 15), SGGSGGSGGS(SEQ ID NO: 17), GGSGG(SEQ ID NO: 19), GSSGSETPGTSESATPESSG(SEQ ID NO: 21), ETPGTSESAT(SEQ ID NO: 23), and GTSES(SEQ ID NO: 25).

In certain embodiments, the amino acid linker that is present at the N-terminus of the inlaid NBE domain comprises GGSGGSGGSGGSGGSGGSGG(SEQ ID NO: 15), and the amino acid linker that is present at the C-terminus of the inlaid NBE domain comprises ETPGTSESAT(SEQ ID NO: 23), GTSES(SEQ ID NO: 25), or is absent.

In certain embodiments, the amino acid linker that is present at the N-terminus of the inlaid NBE domain comprises SGGSGGSGGS(SEQ ID NO: 17), and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker that is present at the N-terminus of the inlaid NBE domain comprises GGSGG(SEQ ID NO: 19), and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker is absent at the N-terminus of the inlaid NBE domain, and the amino acid linker that is present at the C-terminus of the inlaid NBE domain comprises GSSGSETPGTSESATPESSG(SEQ ID NO: 21), ETPGTSESAT(SEQ ID NO: 23), GTSES(SEQ ID NO: 25), or is absent.

In certain embodiments, the amino acid linker that is present at the C-terminus of the inlaid NBE domain comprises GSSGSETPGTSESATPESSG(SEQ ID NO: 21), and the amino acid linker that is present at the N-terminus of the inlaid NBE domain comprises SGGSGGSGGS(SEQ ID NO: 17), GGSGG(SEQ ID NO: 19), or is absent.

In certain embodiments, the amino acid linker that is present at the C-terminus of the inlaid NBE domain comprises ETPGTSESAT(SEQ ID NO: 23), and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

In certain embodiments, the amino acid linker that is present at the C-terminus of the inlaid NBE domain comprises GTSES(SEQ ID NO: 25), and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

In certain embodiments, the amino acid linker is absent at the C-terminus of the inlaid NBE domain, and the amino acid linker that is present at the N-terminus of the inlaid NBE domain comprises GGSGGSGGSGGSGGSGGSGG(SEQ ID NO: 15), SGGSGGSGGS(SEQ ID NO: 17), GGSGG(SEQ ID NO: 19), or is absent.

### Nuclear Localization Signal (NLS)

Any of the Nme2Cas9 proteins described herein (i.e., WT Nme2Cas9, Nme2^{Smu}Cas9, Nme2Cas9 variants, Nme2^{Smu}Cas9 variants, and base editor fusions of the same), may further comprise one or more nuclear localization signals (NLS).

In certain embodiments, the NLS is any one or more of a nucleoplasmin NLS, an SV40 NLS or a C-myc NLS.

In certain embodiments, the NLS comprises an amino acid sequence selected from the group consisting of MKRTADGSEFESPKKKRKV(SEQ ID NO:30), KRTADGSEFEPKKKRKV(SEQ ID NO:31), MKRPAATKKAGQAKKKK(SEQ ID NO:32), KRPAATKKAGQAKKKK(SEQ ID NO:33), MPKKKRKV(SEQ ID NO:34), or PKKKRKV(SEQ ID NO:35).

In certain embodiments, the one or more NLS are positioned at the N-terminus and/or C-terminus of the Nme2Cas9 protein (i.e., WT Nme2Cas9, Nme2^{Smu}Cas9, Nme2Cas9 variant, Nme2^{Smu}Cas9 variant, and base editor fusions of the same).

### HDR And HNH Cas9 Nickases

Cas9 enzymes use their HNH and RuvC domains to cleave the guide-complementary and non-complementary strand of the target DNA, respectively. Cas9 nickases (nCas9s), in which either the HNH or RuvC domain is mutationally inactivated, have been used to induce homology-directed repair (HDR) and to improve genome editing specificity via DSB induction by dual nickases (Mali et al., 2013a; Ran et al., 2013).

Nme2Cas9 nickases include Nme2Cas9^{D16A} (HNH nickase) and Nme2Cas9^{H588A} (RuvC nickase), which possess alanine mutations in catalytic residues of the RuvC and HNH domains, respectively (Esvelt et al., 2013; Hou et al., 2013; Zhang et al., 2013).

### Nme2Cas9 Guide RNA

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); Jinek et al. Science, 337(6096), 816-821 (2012); and Sun et al. Mol. Cell, 76, 938-952 (2019), each of which are incorporated herein by reference.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-26 nucleotides in length (for example, 16, 17, 18, 19, 20, 21, 22, 23, 24, 25, or 26 nucleotides in length), and are at or near the 5′ terminus of a Cas9 gRNA.

As used herein, a “target domain” or “target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015); Sun et al., supra, each incorporated by reference herein). It should be noted that the first and/or second complementarity domains can contain one or more poly-U tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra; Sun et al., supra). A first stem-loop near the 3′ portion of the second complementarity domain is referred to variously as the “proximal domain,” “stem loop 1” (Nishimasu 2014, supra; Nishimasu 2015, supra; Sun et al., supra) and the “nexus” (Briner 2014, supra). One or more additional stem loop structures are generally present near the 3′ end of the gRNA, with the number varying by species: N. meningitidis gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

In certain embodiments, the gRNA comprises: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence.

In certain embodiments, the gRNA comprises at least one modified nucleotide. Chemically modified guide RNAs of the disclosure contain one or more modified nucleotides comprising a modification in a ribose group, a phosphate group, a nucleobase, or a combination thereof.

Chemical modifications to the ribose group may include, but are not limited to, 2′-O-methyl, 2′-fluoro, 2′-deoxy, 2′-O-(2-methoxyethyl) (MOE), 2′-NH₂ (2′-amino), 4′-thio, 2′-O-Allyl, 2′-O-Ethylamine, 2′-O-Cyanoethyl, 2′-O-Acetalester, or a bicyclic nucleotide, such as locked nucleic acid (LNA), 2′-(S)-constrained ethyl (S-cEt), constrained MOE, or 2′-0,4′-C-aminomethylene bridged nucleic acid (2′,4′-BNA^{NC}).

The term “4′-thio” as used herein corresponds to a ribose group modification where the sugar ring oxygen of the ribose is replaced with a sulfur.

Chemical modifications to the phosphate group may include, but are not limited to, a phosphorothioate, phosphonoacetate (PACE), thiophosphonoacetate (thioPACE), amide, triazole, phosphonate, or phosphotriester modification.

Chemical modifications to the nucleobase may include, but are not limited to, 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, or halogenated aromatic groups.

The chemically modified guide RNAs may have one or more chemical modifications in the crRNA portion and/or the tracrRNA portion for a modular or dual RNA guide. The chemically modified guide RNAs may also have one or more chemical modifications in the single guide RNA for the unimolecular guide RNA.

In certain embodiments, the chemically modified Nme2Cas9 gRNA described above further comprises a nucleotide or non-nucleotide loop or linker linking the 3′ end of the crRNA portion to the 5′ end of the tracrRNA portion.

In certain embodiments, the nucleotide loop is chemically modified. In certain embodiments, the nucleotide loop comprises the nucleotide sequence of GAAA. In certain embodiments, the nucleotide loop comprises the nucleotide sequence of (mG)(mA)(mA)(mA), wherein mN corresponds to a 2′-O-methyl RNA and N corresponds to any nucleotide.

In certain embodiments, the non-nucleotide linker comprises an azide linker, an ethylene glycol oligomer, a tetrazine linker, an alkyl chain, a peptide, an amide, or a carbamate (see, e.g., Pils et al. Nucleic Acids Res. 28(9): 1859-1863 (2000)).

In one aspect, the disclosure provides a chemically modified Neisseria meningitidis (Nme) single guide RNA (sgRNA) comprising one or more chemical modifications.

The activity of a guide RNA can be readily determined by any means known in the art. In an embodiment, % activity is measured with the traffic light reporter (TLR) Multi-Cas Variant 1 system (TLR-MCV1), described below. The TLR-MCV1 system will provide a % fluorescent cells which is a measure of % activity.

Nme2Cas9 gRNAs and sgRNAs are described in further detail in WO2023064813, incorporated herein by reference.

### Sequences

TABLE 1
Nme2Cas9 and Nme2Cas9^{Smu} Amino Acid and Nucleic Acid Sequences

| Name | Sequence |
|---|---|
| Nme2Cas9 | MAAFKPNPINYILGLDIGIASVGWAMVEIDEEENPIRLIDLGVRVFE |
| Amino Acid | RAEVPKTGDSLAMARRLARSVRRLTRRRAHRLLRARRLLKREGVL |
| Nme2 PID in | QAADFDENGLIKSLPNTPWQLRAAALDRKLTPLEWSAVLLHLIKHR |
| bold underlined | GYLSQRKNEGETADKELGALLKGVANNAHALQTGDFRTPAELAL |
| text | NKFEKESGHIRNQRGDYSHTFSRKDLQAELILLFEKQKEFGNPHVS |
| | GGLKEGIETLLMTQRPALSGDAVQKMLGHCTFEPAEPKAAKNTYT |
| | AERFIWLTKLNNLRILEQGSERPLTDTERATLMDEPYRKSKLTYAQ |
| | ARKLLGLEDTAFFKGLRYGKDNAEASTLMEMKAYHAISRALEKEG |
| | LKDKKSPLNLSSELQDEIGTAFSLFKTDEDITGRLKDRVQPEILEALL |
| | KHISFDKFVQISLKALRRIVPLMEQGKRYDEACAEIYGDHYGKKNT |
| | EEKIYLPPIPADEIRNPVVLRALSQARKVINGVVRRYGSPARIHIETA |
| | REVGKSFKDRKEIEKRQEENRKDREKAAAKFREYFPNFVGEPKSKD |
| | ILKLRLYEQQHGKCLYSGKEINLVRLNEKGYVEIDHALPFSRTWDD |
| | SFNNKVLVLGSENQNKGNQTPYEYFNGKDNSREWQEFKARVETSR |
| | FPRSKKQRILLQKFDEDGFKECNLNDTRYVNRFLCQFVADHILLTG |
| | KGKRRVFASNGQITNLLRGFWGLRKVRAENDRHHALDAVVVACS |
| | TVAMQQKITRFVRYKEMNAFDGKTIDKETGKVLHQKTHFPQPWEF |
| | FAQEVMIRVFGKPDGKPEFEEADTPEKLRTLLAEKLSSRPEAVHEY |
| | VTPLFVSRAPNRKMSGAHKDTLRSAKRFVKHNEKISVKRVWLTEI |
| | KLADLENMVNYKNGREIELYEALKARLEAYGGNAKQAFDPKDNP |
| | FYKKGGQLVKAVRVEKTQESGVLLNKKNAYTIADNGDMVRVDV |
| | FCKVDKKGKNQYFIVPIYAWQVAENILPDIDCKGYRIDDSYTFC |
| | FSLHKYDLIAFQKDEKSKVEFAYYINCDSSNGRFYLAWHDKGS |
| | KEQQFRISTONLVLIQKYQVNELGKEIRPCRLKKRPPVR (SEQ |
| | ID NO: 1) |
| Nme2Cas9^{Smu} | MAAFKPNPINYILGLDIGIASVGWAMVEIDEEENPIRLIDLGVRVFE |
| Amino Acid | RAEVPKTGDSLAMARRLARSVRRLTRRRAHRLLRARRLLKREGVL |
| Smu PID in | QAADFDENGLIKSLPNTPWQLRAAALDRKLTPLEWSAVLLHLIKHR |
| bold underlined | GYLSQRKNEGETADKELGALLKGVANNAHALQTGDFRTPAELAL |
| text | NKFEKESGHIRNQRGDYSHTFSRKDLQAELILLFEKQKEFGNPHVS |
| | GGLKEGIETLLMTQRPALSGDAVQKMLGHCTFEPAEPKAAKNTYT |
| | AERFIWLTKLNNLRILEQGSERPLTDTERATLMDEPYRKSKLTYAQ |
| | ARKLLGLEDTAFFKGLRYGKDNAEASTLMEMKAYHAISRALEKEG |
| | LKDKKSPLNLSSELQDEIGTAFSLFKTDEDITGRLKDRVQPEILEALL |
| | KHISFDKFVQISLKALRRIVPLMEQGKRYDEACAEIYGDHYGKKNT |
| | EEKIYLPPIPADEIRNPVVLRALSQARKVINGVVRRYGSPARIHIETA |
| | REVGKSFKDRKEIEKRQEENRKDREKAAAKFREYFPNFVGEPKSKD |
| | ILKLRLYEQQHGKCLYSGKEINLVRLNEKGYVEIDHALPFSRTWDD |
| | SFNNKVLVLGSENQNKGNQTPYEYFNGKDNSREWQEFKARVETSR |
| | FPRSKKQRILLQKFDEDGFKECNLNDTRYVNRFLCQFVADHILLTG |
| | KGKRRVFASNGQITNLLRGFWGLRKVRAENDRHHALDAVVVACS |
| | TVAMQQKITRFVRYKEMNAFDGKTIDKETGKVLHQKTHFPQPWEF |
| | FAQEVMIRVFGKPDGKPEFEEADTPEKLRTLLAEKLSSRPEAVHEY |
| | VTPLFVSRAPNRKMSGAHKDTLRSAKRFVKHNEKISVKRVWLTEI |
| | KLADLENMVNYKNGREIELYEALKARLEAYGGNAKQAFDPKDNP |
| | FYKKGGQLVKAVRVEKTQESGVLLNKKNAYTIADNATMVRVDV |
| | YTKAGKNYLVPVYVWQVAQGILPNRAVTSGKSEADWDLIDESF |
| | EFKFSLSRGDLVEMISNKGRIFGYYNGLDRANGSIGIREHDLEK |
| | SKGKDGVHRVGVKTATAFNKYHVDPLGKEIHRCSSEPRPTLKI |
| | KSKK (SEQ ID NO: 2) |
| Nme2Cas9^{Smu} | ATGGCCGCCTTCAAGCCTAACCCAATCAATTACATCCTGGGACT |
| Nucleic Acid | GGACATCGGAATCGCATCCGTGGGATGGGCTATGGTGGAGATC |
| | GACGAGGAGGAGAATCCTATCCGGCTGATCGATCTGGGCGTGA |
| | GAGTGTTTGAGAGGGCCGAGGTGCCAAAGACCGGCGATTCTCTG |
| | GCTATGGCCCGGAGACTGGCACGGAGCGTGAGGCGCCTGACAC |
| | GGAGAAGGGCACACAGGCTGCTGAGGGCACGCCGGCTGCTGAA |
| | GAGAGAGGGCGTGCTGCAGGCAGCAGACTTCGATGAGAATGGC |
| | CTGATCAAGAGCCTGCCAAACACCCCCTGGCAGCTGAGAGCAG |
| | CCGCCCTGGACAGGAAGCTGACACCACTGGAGTGGTCTGCCGTG |
| | CTGCTGCACCTGATCAAGCACCGCGGCTACCTGAGCCAGCGGAA |
| | GAACGAGGGAGAGACAGCAGACAAGGAGCTGGGCGCCCTGCTG |
| | AAGGGAGTGGCCAACAATGCCCACGCCCTGCAGACCGGCGATT |
| | TCAGGACACCTGCCGAGCTGGCCCTGAATAAGTTTGAGAAGGA |
| | GTCCGGCCACATCAGAAACCAGAGGGGCGACTATAGCCACACC |
| | TTCTCCCGCAAGGATCTGCAGGCCGAGCTGATCCTGCTGTTCGA |
| | GAAGCAGAAGGAGTTTGGCAATCCACACGTGAGCGGAGGCCTG |
| | AAGGAGGGAATCGAGACCCTGCTGATGACACAGAGGCCTGCCC |
| | TGTCCGGCGACGCAGTGCAGAAGATGCTGGGACACTGCACCTTC |
| | GAGCCTGCAGAGCCAAAGGCCGCCAAGAACACCTACACAGCCG |
| | AGCGGTTTATCTGGCTGACAAAGCTGAACAATCTGAGAATCCTG |
| | GAGCAGGGATCCGAGAGGCCACTGACCGACACAGAGAGGGCCA |
| | CCCTGATGGATGAGCCTTACCGGAAGTCTAAGCTGACATATGCC |
| | CAGGCCAGAAAGCTGCTGGGCCTGGAGGACACCGCCTTCTTTAA |
| | GGGCCTGAGATACGGCAAGGATAATGCCGAGGCCTCCACACTG |
| | ATGGAGATGAAGGCCTATCACGCCATCTCTCGCGCCCTGGAGAA |
| | GGAGGGCCTGAAGGACAAGAAGTCCCCCCTGAACCTGAGCTCC |
| | GAGCTGCAGGATGAGATCGGCACCGCCTTCTCTCTGTTTAAGAC |
| | CGACGAGGATATCACAGGCCGCCTGAAGGACAGGGTGCAGCCT |
| | GAGATCCTGGAGGCCCTGCTGAAGCACATCTCTTTCGATAAGTT |
| | TGTGCAGATCAGCCTGAAGGCCCTGAGAAGGATCGTGCCACTGA |
| | TGGAGCAGGGCAAGCGGTACGACGAGGCCTGCGCCGAGATCTA |
| | CGGCGATCACTATGGCAAGAAGAACACAGAGGAGAAGATCTAT |
| | CTGCCCCCTATCCCTGCCGACGAGATCAGAAATCCTGTGGTGCT |
| | GAGGGCCCTGTCCCAGGCAAGAAAAGTGATCAACGGAGTGGTG |
| | CGCCGGTACGGATCTCCAGCCCGGATCCACATCGAGACCGCCAG |
| | AGAAGTGGGCAAGAGCTTCAAGGACCGGAAGGAGATCGAGAAG |
| | AGACAGGAGGAGAATCGCAAGGATCGGGAGAAGGCCGCCGCCA |
| | AGTTTAGGGAGTACTTCCCTAACTTTGTGGGCGAGCCAAAGTCT |
| | AAGGACATCCTGAAGCTGCGCCTGTACGAGCAGCAGCACGGCA |
| | AGTGTCTGTATAGCGGCAAGGAGATCAATCTGGTGCGGCTGAAC |
| | GAGAAGGGCTATGTGGAGATCGATCACGCCCTGCCTTTCTCCAG |
| | AACCTGGGACGATTCTTTTAACAATAAGGTGCTGGTGCTGGGCA |
| | GCGAGAACCAGAATAAGGGCAATCAGACACCATACGAGTATTT |
| | CAATGGCAAGGACAACTCCAGGGAGTGGCAGGAGTTCAAGGCC |
| | CGCGTGGAGACCTCTAGATTTCCCAGGAGCAAGAAGCAGCGGA |
| | TCCTGCTGCAGAAGTTCGACGAGGATGGCTTTAAGGAGTGCAAC |
| | CTGAATGACACCAGATACGTGAACCGGTTCCTGTGCCAGTTTGT |
| | GGCCGATCACATCCTGCTGACCGGCAAGGGCAAGAGAAGGGTG |
| | TTCGCCTCTAATGGCCAGATCACAAACCTGCTGAGGGGATTTTG |
| | GGGACTGAGGAAGGTGCGGGCAGAGAATGACAGACACCACGCA |
| | CTGGATGCAGTGGTGGTGGCATGCAGCACCGTGGCAATGCAGC |
| | AGAAGATCACAAGATTCGTGAGGTATAAGGAGATGAACGCCTT |
| | TGACGGCAAGACCATCGATAAGGAGACAGGCAAGGTGCTGCAC |
| | CAGAAGACCCACTTCCCCCAGCCTTGGGAGTTCTTTGCCCAGGA |
| | AGTGATGATCCGGGTGTTCGGCAAGCCAGACGGCAAGCCTGAG |
| | TTTGAGGAGGCCGATACCCCAGAGAAGCTGAGGACACTGCTGG |
| | CAGAGAAGCTGTCTAGCAGGCCAGAGGCAGTGCACGAGTACGT |
| | GACCCCACTGTTCGTGTCCAGGGCACCCAATCGGAAGATGTCTG |
| | GCGCCCACAAGGACACACTGAGAAGCGCCAAGAGGTTTGTGAA |
| | GCACAACGAGAAGATCTCCGTGAAGAGAGTGTGGCTGACCGAG |
| | ATCAAGCTGGCCGATCTGGAGAACATGGTGAATTACAAGAACG |
| | GCAGGGAGATCGAGCTGTATGAGGCCCTGAAGGCAAGGCTGGA |
| | GGCCTACGGAGGAAATGCCAAGCAGGCCTTCGACCCAAAGGAT |
| | AACCCCTTTTATAAGAAGGGAGGACAGCTGGTGAAGGCCGTGC |
| | GGGTGGAGAAGACCCAGGAGAGCGGCGTGCTGCTGAATAAGAA |
| | GAACGCCTACACAATCGCCGACAACGCCACCATGGTGCGGGTG |
| | GACGTGTACACCAAGGCCGGCAAGAACTACCTGGTTCCTGTGTA |
| | CGTGTGGCAGGTGGCCCAGGGCATCTTACCCAACCGCGCCGTGA |
| | CCAGCGGCAAGTCCGAGGCTGACTGGGACCTGATCGATGAGAG |
| | CTTCGAGTTCAAGTTCTCTCTGTCCCGGGGAGATCTCGTGGAAA |
| | TGATCTCCAACAAGGGCAGAATCTTCGGCTACTACAACGGCCTG |
| | GACAGAGCCAACGGCTCTATTGGAATTAGAGAGCACGACCTAG |
| | AGAAGAGCAAGGGCAAAGACGGCGTGCATAGAGTGGGAGTGA |
| | AAACAGCTACAGCATTTAACAAGTACCACGTGGATCCCCTGGGC |
| | AAAGAGATCCACAGATGCAGCAGCGAACCCAGACCTACACTGA |
| | AAATCAAGTCTAAGAAG (SEQ ID NO: 3) |
| Nme2Cas9^{Smu} - | MKRTADGSEFESPKKKRKVEDMAAFKPNPINYILGLDIGIASVGW |
| BPSV40-NLS | AMVEIDEEENPIRLIDLGVRVFERAEVPKTGDSLAMARRLARSVRR |
| Amino Acid | LTRRRAHRLLRARRLLKREGVLQAADFDENGLIKSLPNTPWQLRA |
| NLS sequences | AALDRKLTPLEWSAVLLHLIKHRGYLSQRKNEGETADKELGALLK |
| in bold | GVANNAHALQTGDFRTPAELALNKFEKESGHIRNQRGDYSHTFSR |
| underlined text | KDLQAELILLFEKQKEFGNPHVSGGLKEGIETLLMTQRPALSGDAV |
| “ED” | QKMLGHCTFEPAEPKAAKNTYTAERFIWLTKLNNLRILEQGSERPL |
| amino | TDTERATLMDEPYRKSKLTYAQARKLLGLEDTAFFKGLRYGKDNA |
| acid linkers in | EASTLMEMKAYHAISRALEKEGLKDKKSPLNLSSELQDEIGTAFSLF |
| bold italicized | KTDEDITGRLKDRVQPEILEALLKHISFDKFVQISLKALRRIVPLMEQ |
| text | GKRYDEACAEIYGDHYGKKNTEEKIYLPPIPADEIRNPVVLRALSQ |
| | ARKVINGVVRRYGSPARIHIETAREVGKSFKDRKEIEKRQEENRKD |
| | REKAAAKFREYFPNFVGEPKSKDILKLRLYEQQHGKCLYSGKEINL |
| | VRLNEKGYVEIDHALPFSRTWDDSFNNKVLVLGSENQNKGNQTPY |
| | EYFNGKDNSREWQEFKARVETSRFPRSKKQRILLQKFDEDGFKECN |
| | LNDTRYVNRFLCQFVADHILLTGKGKRRVFASNGQITNLLRGFWG |
| | LRKVRAENDRHHALDAVVVACSTVAMQQKITRFVRYKEMNAFDG |
| | KTIDKETGKVLHQKTHFPQPWEFFAQEVMIRVFGKPDGKPEFEEAD |
| | TPEKLRTLLAEKLSSRPEAVHEYVTPLFVSRAPNRKMSGAHKDTLR |
| | SAKRFVKHNEKISVKRVWLTEIKLADLENMVNYKNGREIELYEAL |
| | KARLEAYGGNAKQAFDPKDNPFYKKGGQLVKAVRVEKTQESGVL |
| | LNKKNAYTIADNATMVRVDVYTKAGKNYLVPVYVWQVAQGILP |
| | NRAVTSGKSEADWDLIDESFEFKFSLSRGDLVEMISNKGRIFGYYN |
| | GLDRANGSIGIREHDLEKSKGKDGVHRVGVKTATAFNKYHVDPLG |
| | KEIHRCSSEPRPTLKIKSKKEDKRTADGSEFEPKKKRKV (SEQ ID |
| | NO: 4) |
| Nme2Cas9^{Smu} - | ATGAAACGGACAGCCGACGGAAGCGAGTTCGAGTCACCAAAGA |
| BPSV40-NLS | AGAAGCGGAAAGTCGAAGATATGGCCGCCTTCAAGCCTAACCC |
| Nucleic Acid | AATCAATTACATCCTGGGACTGGACATCGGAATCGCATCCGTGG |
| | GATGGGCTATGGTGGAGATCGACGAGGAGGAGAATCCTATCCG |
| | GCTGATCGATCTGGGCGTGAGAGTGTTTGAGAGGGCCGAGGTGC |
| | CAAAGACCGGCGATTCTCTGGCTATGGCCCGGAGACTGGCACGG |
| | AGCGTGAGGCGCCTGACACGGAGAAGGGCACACAGGCTGCTGA |
| | GGGCACGCCGGCTGCTGAAGAGAGAGGGCGTGCTGCAGGCAGC |
| | AGACTTCGATGAGAATGGCCTGATCAAGAGCCTGCCAAACACCC |
| | CCTGGCAGCTGAGAGCAGCCGCCCTGGACAGGAAGCTGACACC |
| | ACTGGAGTGGTCTGCCGTGCTGCTGCACCTGATCAAGCACCGCG |
| | GCTACCTGAGCCAGCGGAAGAACGAGGGAGAGACAGCAGACAA |
| | GGAGCTGGGCGCCCTGCTGAAGGGAGTGGCCAACAATGCCCAC |
| | GCCCTGCAGACCGGCGATTTCAGGACACCTGCCGAGCTGGCCCT |
| | GAATAAGTTTGAGAAGGAGTCCGGCCACATCAGAAACCAGAGG |
| | GGCGACTATAGCCACACCTTCTCCCGCAAGGATCTGCAGGCCGA |
| | GCTGATCCTGCTGTTCGAGAAGCAGAAGGAGTTTGGCAATCCAC |
| | ACGTGAGCGGAGGCCTGAAGGAGGGAATCGAGACCCTGCTGAT |
| | GACACAGAGGCCTGCCCTGTCCGGCGACGCAGTGCAGAAGATG |
| | CTGGGACACTGCACCTTCGAGCCTGCAGAGCCAAAGGCCGCCA |
| | AGAACACCTACACAGCCGAGCGGTTTATCTGGCTGACAAAGCTG |
| | AACAATCTGAGAATCCTGGAGCAGGGATCCGAGAGGCCACTGA |
| | CCGACACAGAGAGGGCCACCCTGATGGATGAGCCTTACCGGAA |
| | GTCTAAGCTGACATATGCCCAGGCCAGAAAGCTGCTGGGCCTGG |
| | AGGACACCGCCTTCTTTAAGGGCCTGAGATACGGCAAGGATAAT |
| | GCCGAGGCCTCCACACTGATGGAGATGAAGGCCTATCACGCCAT |
| | CTCTCGCGCCCTGGAGAAGGAGGGCCTGAAGGACAAGAAGTCC |
| | CCCCTGAACCTGAGCTCCGAGCTGCAGGATGAGATCGGCACCGC |
| | CTTCTCTCTGTTTAAGACCGACGAGGATATCACAGGCCGCCTGA |
| | AGGACAGGGTGCAGCCTGAGATCCTGGAGGCCCTGCTGAAGCA |
| | CATCTCTTTCGATAAGTTTGTGCAGATCAGCCTGAAGGCCCTGA |
| | GAAGGATCGTGCCACTGATGGAGCAGGGCAAGCGGTACGACGA |
| | GGCCTGCGCCGAGATCTACGGCGATCACTATGGCAAGAAGAAC |
| | ACAGAGGAGAAGATCTATCTGCCCCCTATCCCTGCCGACGAGAT |
| | CAGAAATCCTGTGGTGCTGAGGGCCCTGTCCCAGGCAAGAAAA |
| | GTGATCAACGGAGTGGTGCGCCGGTACGGATCTCCAGCCCGGAT |
| | CCACATCGAGACCGCCAGAGAAGTGGGCAAGAGCTTCAAGGAC |
| | CGGAAGGAGATCGAGAAGAGACAGGAGGAGAATCGCAAGGAT |
| | CGGGAGAAGGCCGCCGCCAAGTTTAGGGAGTACTTCCCTAACTT |
| | TGTGGGCGAGCCAAAGTCTAAGGACATCCTGAAGCTGCGCCTGT |
| | ACGAGCAGCAGCACGGCAAGTGTCTGTATAGCGGCAAGGAGAT |
| | CAATCTGGTGCGGCTGAACGAGAAGGGCTATGTGGAGATCGAT |
| | CACGCCCTGCCTTTCTCCAGAACCTGGGACGATTCTTTTAACAAT |
| | AAGGTGCTGGTGCTGGGCAGCGAGAACCAGAATAAGGGCAATC |
| | AGACACCATACGAGTATTTCAATGGCAAGGACAACTCCAGGGA |
| | GTGGCAGGAGTTCAAGGCCCGCGTGGAGACCTCTAGATTTCCCA |
| | GGAGCAAGAAGCAGCGGATCCTGCTGCAGAAGTTCGACGAGGA |
| | TGGCTTTAAGGAGTGCAACCTGAATGACACCAGATACGTGAACC |
| | GGTTCCTGTGCCAGTTTGTGGCCGATCACATCCTGCTGACCGGC |
| | AAGGGCAAGAGAAGGGTGTTCGCCTCTAATGGCCAGATCACAA |
| | ACCTGCTGAGGGGATTTTGGGGACTGAGGAAGGTGCGGGCAGA |
| | GAATGACAGACACCACGCACTGGATGCAGTGGTGGTGGCATGC |
| | AGCACCGTGGCAATGCAGCAGAAGATCACAAGATTCGTGAGGT |
| | ATAAGGAGATGAACGCCTTTGACGGCAAGACCATCGATAAGGA |
| | GACAGGCAAGGTGCTGCACCAGAAGACCCACTTCCCCCAGCCTT |
| | GGGAGTTCTTTGCCCAGGAAGTGATGATCCGGGTGTTCGGCAAG |
| | CCAGACGGCAAGCCTGAGTTTGAGGAGGCCGATACCCCAGAGA |
| | AGCTGAGGACACTGCTGGCAGAGAAGCTGTCTAGCAGGCCAGA |
| | GGCAGTGCACGAGTACGTGACCCCACTGTTCGTGTCCAGGGCAC |
| | CCAATCGGAAGATGTCTGGCGCCCACAAGGACACACTGAGAAG |
| | CGCCAAGAGGTTTGTGAAGCACAACGAGAAGATCTCCGTGAAG |
| | AGAGTGTGGCTGACCGAGATCAAGCTGGCCGATCTGGAGAACA |
| | TGGTGAATTACAAGAACGGCAGGGAGATCGAGCTGTATGAGGC |
| | CCTGAAGGCAAGGCTGGAGGCCTACGGAGGAAATGCCAAGCAG |
| | GCCTTCGACCCAAAGGATAACCCCTTTTATAAGAAGGGAGGACA |
| | GCTGGTGAAGGCCGTGCGGGTGGAGAAGACCCAGGAGAGCGGC |
| | GTGCTGCTGAATAAGAAGAACGCCTACACAATCGCCGACAACG |
| | CCACCATGGTGCGGGTGGACGTGTACACCAAGGCCGGCAAGAA |
| | CTACCTGGTTCCTGTGTACGTGTGGCAGGTGGCCCAGGGCATCT |
| | TACCCAACCGCGCCGTGACCAGCGGCAAGTCCGAGGCTGACTG |
| | GGACCTGATCGATGAGAGCTTCGAGTTCAAGTTCTCTCTGTCCC |
| | GGGGAGATCTCGTGGAAATGATCTCCAACAAGGGCAGAATCTTC |
| | GGCTACTACAACGGCCTGGACAGAGCCAACGGCTCTATTGGAAT |
| | TAGAGAGCACGACCTAGAGAAGAGCAAGGGCAAAGACGGCGTG |
| | CATAGAGTGGGAGTGAAAACAGCTACAGCATTTAACAAGTACC |
| | ACGTGGATCCCCTGGGCAAAGAGATCCACAGATGCAGCAGCGA |
| | ACCCAGACCTACACTGAAAATCAAGTCTAAGAAGGAGGATAAA |
| | AGAACCGCCGACGGCAGCGAATTCGAGCCCAAGAAGAAGAGGA |
| | AAGTC (SEQ ID NO: 5) |
| Nme2^{Smu}- | MAAFKPNPINYILGLAIGIASVGWAMVEIDEEENPIRLIDLGVRVFE |
| ABE8e-i1 | RAEVPKTGDSLAMARRLARSVRRLTRRRAHRLLRARRLLKREGVL |
| Amino Acid | QAADFDENGLIKSLPNTPWQLRAAALDRKLTPLEWSAVLLHLIKHR |
| TadA8e | GYLSQRKNEGETADKELGALLKGVANNAHALQTGDFRTPAELAL |
| sequence in | NKFEKESGHIRNQRGDYSHTFSRKDLQAELILLFEKQKEFGNPHVS |
| bold underlined | GGLKEGIETLLMTQRPALSGDAVQKMLGHCTFEPAEPKAAKNTYT |
| text | AERFIWLTKLNNLRILEQXₙSEVEFSHEYWMRHALTLAKRARDER |
| “Xₙ” amino | EVPVGAVLVLNNRVIGEGWNRAIGLHDPTAHAEIMALRQGGL |
| acid linkers in | VMQNYRLIDATLYVTFEPCVMCAGAMIHSRIGRVVFGVRNSKR |
| bold italicized | GAAGSLMNVLNYPGMNHRVEITEGILADECAALLCDFYRMPR |
| text, wherein | QVFNAQKKAQSSINXₙGSERPLTDTERATLMDEPYRKSKLTYAQA |
| “X” | RKLLGLEDTAFFKGLRYGKDNAEASTLMEMKAYHAISRALEKEGL |
| corresponds to | KDKKSPLNLSSELQDEIGTAFSLFKTDEDITGRLKDRVQPEILEALLK |
| any amino acid | HISFDKFVQISLKALRRIVPLMEQGKRYDEACAEIYGDHYGKKNTE |
| and “n” | EKIYLPPIPADEIRNPVVLRALSQARKVINGVVRRYGSPARIHIETAR |
| corresponds to | EVGKSFKDRKEIEKRQEENRKDREKAAAKFREYFPNFVGEPKSKDI |
| an integer of | LKLRLYEQQHGKCLYSGKEINLVRLNEKGYVEIDHALPFSRTWDDS |
| between 0 and | FNNKVLVLGSENQNKGNQTPYEYFNGKDNSREWQEFKARVETSRF |
| 20. When “n” is | PRSKKQRILLQKFDEDGFKECNLNDTRYVNRFLCQFVADHILLTGK |
| 0, the amino | GKRRVFASNGQITNLLRGFWGLRKVRAENDRHHALDAVVVACST |
| acid linker “X” | VAMQQKITRFVRYKEMNAFDGKTIDKETGKVLHQKTHFPQPWEFF |
| is absent. | AQEVMIRVFGKPDGKPEFEEADTPEKLRTLLAEKLSSRPEAVHEYV |
| | TPLFVSRAPNRKMSGAHKDTLRSAKRFVKHNEKISVKRVWLTEIKL |
| | ADLENMVNYKNGREIELYEALKARLEAYGGNAKQAFDPKDNPFY |
| | KKGGQLVKAVRVEKTQESGVLLNKKNAYTIADNATMVRVDVYTK |
| | AGKNYLVPVYVWQVAQGILPNRAVTSGKSEADWDLIDESFEFKFS |
| | LSRGDLVEMISNKGRIFGYYNGLDRANGSIGIREHDLEKSKGKDGV |
| | HRVGVKTATAFNKYHVDPLGKEIHRCSSEPRPTLKIKSKK (SEQ ID |
| | NO: 6) |
| Nme2^{Smu}- | ATGGCCGCCTTCAAGCCTAACCCAATCAATTACATCCTGGGACT |
| ABE8e-i1 | GGCCATCGGAATCGCATCCGTGGGATGGGCTATGGTGGAGATCG |
| Nucleic Acid | ACGAGGAGGAGAATCCTATCCGGCTGATCGATCTGGGCGTGAG |
| “X” nucleic | AGTGTTTGAGAGGGCCGAGGTGCCAAAGACCGGCGATTCTCTGG |
| acid sequence | CTATGGCCCGGAGACTGGCACGGAGCGTGAGGCGCCTGACACG |
| encoding the | GAGAAGGGCACACAGGCTGCTGAGGGCACGCCGGCTGCTGAAG |
| amino acid | AGAGAGGGCGTGCTGCAGGCAGCAGACTTCGATGAGAATGGCC |
| linkers | TGATCAAGAGCCTGCCAAACACCCCCTGGCAGCTGAGAGCAGC |
| | CGCCCTGGACAGGAAGCTGACACCACTGGAGTGGTCTGCCGTGC |
| | TGCTGCACCTGATCAAGCACCGCGGCTACCTGAGCCAGCGGAAG |
| | AACGAGGGAGAGACAGCAGACAAGGAGCTGGGCGCCCTGCTGA |
| | AGGGAGTGGCCAACAATGCCCACGCCCTGCAGACCGGCGATTTC |
| | AGGACACCTGCCGAGCTGGCCCTGAATAAGTTTGAGAAGGAGT |
| | CCGGCCACATCAGAAACCAGAGGGGCGACTATAGCCACACCTT |
| | CTCCCGCAAGGATCTGCAGGCCGAGCTGATCCTGCTGTTCGAGA |
| | AGCAGAAGGAGTTTGGCAATCCACACGTGAGCGGAGGCCTGAA |
| | GGAGGGAATCGAGACCCTGCTGATGACACAGAGGCCTGCCCTG |
| | TCCGGCGACGCAGTGCAGAAGATGCTGGGACACTGCACCTTCGA |
| | GCCTGCAGAGCCAAAGGCCGCCAAGAACACCTACACAGCCGAG |
| | CGGTTTATCTGGCTGACAAAGCTGAACAATCTGAGAATCCTGGA |
| | GCAGXTCTGAGGTGGAGTTTTCCCACGAGTACTGGATGAGACAT |
| | GCCCTGACCCTGGCCAAGAGGGCACGCGATGAGAGGGAGGTGC |
| | CTGTGGGAGCCGTGCTGGTGCTGAACAATAGAGTGATCGGCGA |
| | GGGCTGGAACAGAGCCATCGGCCTGCACGACCCAACAGCCCAT |
| | GCCGAAATTATGGCCCTGAGACAGGGCGGCCTGGTCATGCAGA |
| | ACTACAGACTGATTGACGCCACCCTGTACGTGACATTCGAGCCT |
| | TGCGTGATGTGCGCCGGCGCCATGATCCACTCTAGGATCGGCCG |
| | CGTGGTGTTTGGCGTGAGGAACAGCAAACGGGGCGCCGCAGGC |
| | TCCCTGATGAACGTGCTGAACTACCCCGGCATGAATCACCGCGT |
| | CGAAATTACCGAGGGAATCCTGGCAGATGAATGTGCCGCCCTGC |
| | TGTGCGACTTCTACCGGATGCCTAGACAGGTGTTCAATGCTCAG |
| | AAGAAGGCCCAGAGCTCCATCAACXCGGATCCGAGAGGCCACT |
| | GACCGACACAGAGAGGGCCACCCTGATGGATGAGCCTTACCGG |
| | AAGTCTAAGCTGACATATGCCCAGGCCAGAAAGCTGCTGGGCCT |
| | GGAGGACACCGCCTTCTTTAAGGGCCTGAGATACGGCAAGGAT |
| | AATGCCGAGGCCTCCACACTGATGGAGATGAAGGCCTATCACGC |
| | CATCTCTCGCGCCCTGGAGAAGGAGGGCCTGAAGGACAAGAAG |
| | TCCCCCCTGAACCTGAGCTCCGAGCTGCAGGATGAGATCGGCAC |
| | CGCCTTCTCTCTGTTTAAGACCGACGAGGATATCACAGGCCGCC |
| | TGAAGGACAGGGTGCAGCCTGAGATCCTGGAGGCCCTGCTGAA |
| | GCACATCTCTTTCGATAAGTTTGTGCAGATCAGCCTGAAGGCCC |
| | TGAGAAGGATCGTGCCACTGATGGAGCAGGGCAAGCGGTACGA |
| | CGAGGCCTGCGCCGAGATCTACGGCGATCACTATGGCAAGAAG |
| | AACACAGAGGAGAAGATCTATCTGCCCCCTATCCCTGCCGACGA |
| | GATCAGAAATCCTGTGGTGCTGAGGGCCCTGTCCCAGGCAAGAA |
| | AAGTGATCAACGGAGTGGTGCGCCGGTACGGATCTCCAGCCCG |
| | GATCCACATCGAGACCGCCAGAGAAGTGGGCAAGAGCTTCAAG |
| | GACCGGAAGGAGATCGAGAAGAGACAGGAGGAGAATCGCAAG |
| | GATCGGGAGAAGGCCGCCGCCAAGTTTAGGGAGTACTTCCCTAA |
| | CTTTGTGGGCGAGCCAAAGTCTAAGGACATCCTGAAGCTGCGCC |
| | TGTACGAGCAGCAGCACGGCAAGTGTCTGTATAGCGGCAAGGA |
| | GATCAATCTGGTGCGGCTGAACGAGAAGGGCTATGTGGAGATC |
| | GATCACGCCCTGCCTTTCTCCAGAACCTGGGACGATTCTTTTAAC |
| | AATAAGGTGCTGGTGCTGGGCAGCGAGAACCAGAATAAGGGCA |
| | ATCAGACACCATACGAGTATTTCAATGGCAAGGACAACTCCAGG |
| | GAGTGGCAGGAGTTCAAGGCCCGCGTGGAGACCTCTAGATTTCC |
| | CAGGAGCAAGAAGCAGCGGATCCTGCTGCAGAAGTTCGACGAG |
| | GATGGCTTTAAGGAGTGCAACCTGAATGACACCAGATACGTGA |
| | ACCGGTTCCTGTGCCAGTTTGTGGCCGATCACATCCTGCTGACC |
| | GGCAAGGGCAAGAGAAGGGTGTTCGCCTCTAATGGCCAGATCA |
| | CAAACCTGCTGAGGGGATTTTGGGGACTGAGGAAGGTGCGGGC |
| | AGAGAATGACAGACACCACGCACTGGATGCAGTGGTGGTGGCA |
| | TGCAGCACCGTGGCAATGCAGCAGAAGATCACAAGATTCGTGA |
| | GGTATAAGGAGATGAACGCCTTTGACGGCAAGACCATCGATAA |
| | GGAGACAGGCAAGGTGCTGCACCAGAAGACCCACTTCCCCCAG |
| | CCTTGGGAGTTCTTTGCCCAGGAAGTGATGATCCGGGTGTTCGG |
| | CAAGCCAGACGGCAAGCCTGAGTTTGAGGAGGCCGATACCCCA |
| | GAGAAGCTGAGGACACTGCTGGCAGAGAAGCTGTCTAGCAGGC |
| | CAGAGGCAGTGCACGAGTACGTGACCCCACTGTTCGTGTCCAGG |
| | GCACCCAATCGGAAGATGTCTGGCGCCCACAAGGACACACTGA |
| | GAAGCGCCAAGAGGTTTGTGAAGCACAACGAGAAGATCTCCGT |
| | GAAGAGAGTGTGGCTGACCGAGATCAAGCTGGCCGATCTGGAG |
| | AACATGGTGAATTACAAGAACGGCAGGGAGATCGAGCTGTATG |
| | AGGCCCTGAAGGCAAGGCTGGAGGCCTACGGAGGAAATGCCAA |
| | GCAGGCCTTCGACCCAAAGGATAACCCCTTTTATAAGAAGGGAG |
| | GACAGCTGGTGAAGGCCGTGCGGGTGGAGAAGACCCAGGAGAG |
| | CGGCGTGCTGCTGAATAAGAAGAACGCCTACACAATCGCCGAC |
| | AACGCCACCATGGTGCGGGTGGACGTGTACACCAAGGCCGGCA |
| | AGAACTACCTGGTTCCTGTGTACGTGTGGCAGGTGGCCCAGGGC |
| | ATCTTACCCAACCGCGCCGTGACCAGCGGCAAGTCCGAGGCTGA |
| | CTGGGACCTGATCGATGAGAGCTTCGAGTTCAAGTTCTCTCTGT |
| | CCCGGGGAGATCTCGTGGAAATGATCTCCAACAAGGGCAGAAT |
| | CTTCGGCTACTACAACGGCCTGGACAGAGCCAACGGCTCTATTG |
| | GAATTAGAGAGCACGACCTAGAGAAGAGCAAGGGCAAAGACG |
| | GCGTGCATAGAGTGGGAGTGAAAACAGCTACAGCATTTAACAA |
| | GTACCACGTGGATCCCCTGGGCAAAGAGATCCACAGATGCAGC |
| | AGCGAACCCAGACCTACACTGAAAATCAAGTCTAAGAAG (SEQ |
| | ID NO: 7) |
| Nme2^{Smu}- | MAAFKPNPINYILGLAIGIASVGWAMVEIDEEENPIRLIDLGVRVFE |
| ABE8e-i8 | RAEVPKTGDSLAMARRLARSVRRLTRRRAHRLLRARRLLKREGVL |
| Amino Acid | QAADFDENGLIKSLPNTPWQLRAAALDRKLTPLEWSAVLLHLIKHR |
| TadA8e | GYLSQRKNEGETADKELGALLKGVANNAHALQTGDFRTPAELAL |
| sequence in | NKFEKESGHIRNQRGDYSHTFSRKDLQAELILLFEKQKEFGNPHVS |
| bold underlined | GGLKEGIETLLMTQRPALSGDAVQKMLGHCTFEPAEPKAAKNTYT |
| text | AERFIWLTKLNNLRILEQGSERPLTDTERATLMDEPYRKSKLTYAQ |
| “Xₙ” amino | ARKLLGLEDTAFFKGLRYGKDNAEASTLMEMKAYHAISRALEKEG |
| acid linkers in | LKDKKSPLNLSSELQDEIGTAFSLFKTDEDITGRLKDRVQPEILEALL |
| bold italicized | KHISFDKFVQISLKALRRIVPLMEQGKRYDEACAEIYGDHYGKKNT |
| text, wherein | EEKIYLPPIPADEIRNPVVLRALSQARKVINGVVRRYGSPARIHIETA |
| “X” | REVGKSFKDRKEIEKRQEENRKDREKAAAKFREYFPNFVGEPKSKD |
| corresponds to | ILKLRLYEQQHGKCLYSGKEINLVRLNEKGYVEIDHALPFSRTWDD |
| any amino acid | SFNNKVLVLGSENQNKGNQTPYEYFNGKDNSREWQEFKARVETSR |
| and ″n″ | FPRSKKQRILLQKFDEDGFKECNLNDTRYVNRFLCQFVADHILLTG |
| corresponds to | KGKRRVFASNGQITNLLRGFWGLRKVRAENDRHHALDAVVVACS |
| an integer of | TVAMQQKITRFVRYKEMNAFDGKTIDKETGKVLHQKTHFPQPWEF |
| between 0 and | FAQEVMIRVFGKPDGKPXₙSEVEFSHEYWMRHALTLAKRARDER |
| 20. When “n” is | EVPVGAVLVLNNRVIGEGWNRAIGLHDPTAHAEIMALRQGGL |
| 0, the amino | VMQNYRLIDATLYVTFEPCVMCAGAMIHSRIGRVVFGVRNSKR |
| acid linker “X” | GAAGSLMNVLNYPGMNHRVEITEGILADECAALLCDFYRMPR |
| is absent. | QVFNAQKKAQSSINXₙEFEEADTPEKLRTLLAEKLSSRPEAVHEYV |
| | TPLFVSRAPNRKMSGAHKDTLRSAKRFVKHNEKISVKRVWLTEIKL |
| | ADLENMVNYKNGREIELYEALKARLEAYGGNAKQAFDPKDNPFY |
| | KKGGQLVKAVRVEKTQESGVLLNKKNAYTIADNATMVRVDVYTK |
| | AGKNYLVPVYVWQVAQGILPNRAVTSGKSEADWDLIDESFEFKFS |
| | LSRGDLVEMISNKGRIFGYYNGLDRANGSIGIREHDLEKSKGKDGV |
| | HRVGVKTATAFNKYHVDPLGKEIHRCSSEPRPTLKIKSKK (SEQ ID |
| | NO: 8) |

TABLE 2
Base Editor Amino Acid and Nucleic Acid Sequences

| Name | Sequence |
|---|---|
| TadA8e | SEVEFSHEYWMRHALTLAKRARDEREVPVGAVLVLNNRVIGEGW |
| Amino Acid | NRAIGLHDPTAHAEIMALRQGGLVMQNYRLIDATLYVTFEPCVMC |
| | AGAMIHSRIGRVVFGVRNSKRGAAGSLMNVLNYPGMNHRVEITEG |
| | ILADECAALLCDFYRMPRQVFNAQKKAQSSIN (SEQ ID NO: 9) |
| TadA8e | TCTGAGGTGGAGTTTTCCCACGAGTACTGGATGAGACATGCCCT |
| Nucleic Acid | GACCCTGGCCAAGAGGGCACGCGATGAGAGGGAGGTGCCTGTG |
| | GGAGCCGTGCTGGTGCTGAACAATAGAGTGATCGGCGAGGGCT |
| | GGAACAGAGCCATCGGCCTGCACGACCCAACAGCCCATGCCGA |
| | AATTATGGCCCTGAGACAGGGCGGCCTGGTCATGCAGAACTACA |
| | GACTGATTGACGCCACCCTGTACGTGACATTCGAGCCTTGCGTG |
| | ATGTGCGCCGGCGCCATGATCCACTCTAGGATCGGCCGCGTGGT |
| | GTTTGGCGTGAGGAACAGCAAACGGGGCGCCGCAGGCTCCCTG |
| | ATGAACGTGCTGAACTACCCCGGCATGAATCACCGCGTCGAAAT |
| | TACCGAGGGAATCCTGGCAGATGAATGTGCCGCCCTGCTGTGCG |
| | ACTTCTACCGGATGCCTAGACAGGTGTTCAATGCTCAGAAGAAG |
| | GCCCAGAGCTCCATCAAC (SEQ ID NO: 10) |
| rAPOBEC | SSETGPVAVDPTLRRRIEPHEFEVFFDPRELRKETCLLYEINWGGRH |
| Amino Acid | SIWRHTSQNTNKHVEVNFIEKFTTERYFCPNTRCSITWFLSWSPCGE |
| | CSRAITEFLSRYPHVTLFIYIARLYHHADPRNRQGLRDLISSGVTIQI |
| | MTEQESGYCWRNFVNYSPSNEAHWPRYPHLWVRLYVLELYCIILG |
| | LPPCLNILRRKQPQLTFFTIALQSCHYQRLPPHILWATGLK (SEQ ID |
| | NO: 11) |
| rAPOBEC | AGCTCAGAGACTGGCCCAGTGGCTGTGGACCCCACATTGAGACG |
| Nucleic Acid | GCGGATCGAGCCCCATGAGTTTGAGGTATTCTTCGATCCGAGAG |
| | AGCTCCGCAAGGAGACCTGCCTGCTTTACGAAATTAATTGGGGG |
| | GGCCGGCACTCCATTTGGCGACATACATCACAGAACACTAACAA |
| | GCACGTCGAAGTCAACTTCATCGAGAAGTTCACGACAGAAAGA |
| | TATTTCTGTCCGAACACAAGGTGCAGCATTACCTGGTTTCTCAGC |
| | TGGAGCCCATGCGGCGAATGTAGTAGGGCCATCACTGAATTCCT |
| | GTCAAGGTATCCCCACGTCACTCTGTTTATTTACATCGCAAGGCT |
| | GTACCACCACGCTGACCCCCGCAATCGACAAGGCCTGCGGGATT |
| | TGATCTCTTCAGGTGTGACTATCCAAATTATGACTGAGCAGGAG |
| | TCAGGATACTGCTGGAGAAACTTTGTGAATTATAGCCCGAGTAA |
| | TGAAGCCCACTGGCCTAGGTATCCCCATCTGTGGGTACGACTGT |
| | ACGTTCTTGAACTGTACTGCATCATACTGGGCCTGCCTCCTTGTC |
| | TCAACATTCTGAGAAGGAAGCAGCCACAGCTGACATTCTTTACC |
| | ATCGCTCTTCAGTCTTGTCATTACCAGCGACTGCCCCCACACATT |
| | CTCTGGGCCACCGGGTTGAAA (SEQ ID NO: 12) |
| evoFERNY | FERNYDPRELRKETYLLYEIKWGKSGKLWRHWCQNNRTQHAEVY |
| Amino Acid | FLENIFNARRFNPSTHCSITWYLSWSPCAECSQKIVDFLKEHPNVNL |
| | EIYVARLYYPENERNRQGLRDLVNSGVTIRIMDLPDYNYCWKTFVS |
| | DQGGDEDYWPGHFAPWIKQYSLKL (SEQ ID NO: 13) |
| evoFERNY | TTTGAGAGGAACTACGACCCCCGGGAGCTGAGAAAGGAGACAT |
| Nucleic Acid | ACCTGCTGTATGAGATCAAGTGGGGCAAGTCCGGCAAGCTGTGG |
| | AGGCACTGGTGCCAGAACAATCGCACACAGCACGCCGAGGTGT |
| | ACTTCCTGGAGAACATCTTTAATGCCCGGAGATTCAATCCATCT |
| | ACCCACTGTAGCATCACATGGTATCTGAGCTGGTCCCCCTGCGC |
| | CGAGTGTTCTCAGAAGATCGTGGATTTCCTGAAGGAGCACCCTA |
| | ACGTGAATCTGGAGATCTATGTGGCCCGGCTGTACTATCCAGAG |
| | AACGAGAGGAATAGGCAGGGCCTGCGGGATCTGGTGAATTCCG |
| | GCGTGACCATCAGAATCATGGACCTGCCAGATTACAACTATTGC |
| | TGGAAGACCTTCGTGAGCGATCAGGGAGGCGACGAGGATTACT |
| | GGCCAGGACACTTCGCCCCTTGGATCAAGCAGTATAGCCTGAAG |
| | CTG (SEQ ID NO: 14) |

TABLE 3
Linker Amino Acid and Nucleic Acid Sequences

| Name | Sequence |
|---|---|
| “GGS” linker - | GGSGGSGGSGGSGGSGGSGG (SEQ ID NO: 15) |
| 20 amino acids | |
| Amino Acid | |
| “GGS” linker - | GGCGGATCAGGAGGCTCTGGCGGTTCAGGTGGATCAGGCGGTA |
| 20 amino acids | GCGGAGGTTCAGGTGGT (SEQ ID NO: 16) |
| Nucleic Acid | |
| “GGS” linker - | SGGSGGSGGS (SEQ ID NO: 17) |
| 10 amino acids | |
| Amino Acid | |
| “GGS” linker - | TCTGGCGGTTCAGGTGGATCAGGCGGTAGC (SEQ ID NO: 18) |
| 10 amino acids | |
| Nucleic Acid | |
| “GGS” linker - | GGSGG (SEQ ID NO: 19) |
| 5 amino acids | |
| Amino Acid | |
| “GGS” linker - | GGCGGTTCAGGTGGA (SEQ ID NO: 20) |
| 5 amino acids | |
| Nucleic Acid | |
| “SES” linker - | GSSGSETPGTSESATPESSG (SEQ ID NO: 21) |
| 20 amino acids | |
| Amino Acid | |
| “SES” linker - | GGCTCCTCTGGCTCTGAGACACCTGGCACAAGCGAGAGCGCAAC |
| 20 amino acids | ACCTGAAAGCAGCGGC (SEQ ID NO: 22) |
| Nucleic Acid | |
| “SES” linker - | ETPGTSESAT (SEQ ID NO: 23) |
| 10 amino acids | |
| Amino Acid | |
| “SES” linker - | GAGACACCTGGCACAAGCGAGAGCGCAACA (SEQ ID NO: 24) |
| 10 amino acids | |
| Nucleic Acid | |
| “SES” linker - | GTSES (SEQ ID NO: 25) |
| 5 amino acids | |
| Amino Acid | |
| “SES” linker - | GGCACAAGCGAGAGC (SEQ ID NO: 26) |
| 5 amino acids | |
| Nucleic Acid | |

### EXAMPLES

While several experimental Examples are contemplated, these Examples are intended to be non-limiting.

### Example I

### Material and Method

### Molecular Cloning

Nucleotide sequences of Nme2Cas9 and Nme2^{Smu}Cas9 base editors are provided in Table 1 and FIG. 25. Plasmids expressing Nme2-ABE variants were constructed by Gibson assembly using Addgene plasmid #122610 as a backbone containing the CMV promoter and N- and C-terminal BP-SV40 NLSs. To generate Nme2-ABE-nt, the open reading frame of the N-terminally fused Nme2-ABE (see Zhang, H. et al. Adenine base editing in vivo with a single adeno-associated virus vector. GEN Biotechnol. 1, 285-299 (2022)) was PCR-amplified and cloned into the CMV backbone. The domain-inlaid Nme2-ABEs were constructed with two sequential assemblies: first, nNme2D16A was assembled into the CMV backbone, and second, a gene block encoding the TadA8e domain and linkers was assembled into the assigned insertion sites. The domain-inlaid CBE deaminases were cloned in similar fashion to the ABE constructs, with Addgene #122610 as a backbone containing the CMV promoter, terminal BP-SV40 NLSs and a single UGI domain, with gene blocks encoding the evoFERNY (see Thuronyi, B. W. et al. Continuous evolution of base editors with expanded target compatibility and improved activity. Nat Biotechnol 37, 1070-1079 (2019)) or rAPOBEC1 (rA1) (see Komor, A. C., Kim, Y. B., Packer, M. S., Zuris, J. A. & Liu, D. R. Programmable editing of a target base in genomic DNA without double-stranded DNA cleavage. Nature 533, 420-424 (2016)). deaminase. Nme2-evoFERNY-nt was constructed via Gibson assembly by replacing nSpyD10A (Addgene #122610) with nNme2D16A and removing one of the UGI domains. Nme2-rAl-nt was subsequently cloned by replacing the evoFERNY domain with rA1 using the Nme2-evoFERNY-nt plasmid. Nme2-ABE-i1^{V106W} was cloned by site-directed mutagenesis (SDM), using NEB's KLD enzyme mix (NEB #M0554S) with the appropriate Nme2Cas9 effector plasmid as a template. The nSauCas9D10A plasmid used for the orthogonal R-loop assay was also cloned by SDM using CMV-dSauCas9 (Addgene #138162) as a template. U6-driven sgRNA plasmids for the various Cas effectors were cloned using pBluescript sgRNA expression plasmids (Addgene #122089, #122090, #122091 for SpyCas9, SauCas9 and Nme2Cas9 respectively). In brief, the sgRNA plasmids were digested with BfuAI, followed by Gibson assembly with ssDNA bridge oligos containing a spacer of interest (G/N23 for Nme2Cas9, G/N19 for SpyCas9 and G/N21 for SauCas9). Nme2^{Smu}-ABE variants were cloned by replacing the Nme2Cas9 PID with the SmuCas9 PID using a gene block and Gibson assembly. The single-vector AAV plasmids were cloned by replacing the Nme2-ABE effector from AAV-Nme2-ABE8e_V2 μlasmid as described by Zhang (see Zhang, H. et al. Adenine base editing in vivo with a single adeno-associated virus vector. GEN Biotechnol. 1, 285-299 (2022)). with the described domain-inlaid variants.

In Vitro mRNA Synthesis

mRNAs used in this manuscript were in vitro transcribed as described by Zhang (see Zhang, H. et al. Adenine base editing in vivo with a single adeno-associated virus vector. GEN Biotechnol. 1, 285-299 (2022)), using the Hiscribe T7 RNA synthesis kit (NEB #E2040S). In brief, 500 ng of linearized plasmid template was used for the reaction, with complete substitution of uridine to 1-methylpseudouridine and CleanCap AG analog (N-1081 and N-7113, TriLink Biotechnologies).

### Transient Transfection

Mouse N2A (ATCC #CCL-131), HEK293T (ATCC #CRL-3216) cells and their reporter-transduced derivatives were cultured in Dulbecco's Modified Eagle's Medium (DMEM; Genesee Scientific #25-500) supplemented with 10% fetal bovine serum (FBS; Gibco #26140079). All cells were incubated at 37° C. with 5% CO2. For plasmid transfections, cells were seeded in 96-well plates at−15,000 cells per well and incubated overnight. The following day, cells were transfected with plasmid DNA using Lipofectamine 2000 (ThermoFisher #11668019) following the manufacturer's protocol. For editing the mCherry reporter and endogenous target sites, 100 ng of effector plasmid and 100 ng of sgRNA plasmid was transfected with 0.75 μl Lipofectamine 2000. For the orthogonal R-loop assay, 125 ng of each effector and each sgRNA was used with 0.75 μl Lipofectamine 2000. For editing experiments with amplicon sequencing analysis, genomic DNA was extracted from cells 72 h post-transfection with QuickExtract (Lucigen #QE0905) following the manufacturer's protocol.

### Electroporation

Rett syndrome PDFs were obtained from the Rett Syndrome Research Trust and cultured in Dulbecco's Modified Eagle's Medium (DMEM; Genesee Scientific #25-500) supplemented with 15% fetal bovine serum (Gibco #26140079) and 1× nonessential amino acids (Gibco #11140050). These cells were also incubated at 37° C. with 5% CO2. PDF electroporation's were performed using the Neon Transfection System 10 μl kit (ThermoFisher #MPK1096) as described by Zang (see Zhang, H. et al. Adenine base editing in vivo with a single adeno-associated virus vector. GEN Biotechnol. 1, 285-299 (2022)). A total of 500 ng ABE mRNA and 100 pmol sgRNA were electroporated into−50,000 PDF cells. 48 h post-electroporation, genomic DNA was extracted with QuickExtract (Lucigen #QE09050) for amplicon sequencing.

### Flow Cytometry

In total, 72 h post-transfection, cells were trypsinized, collected, and washed with FACS buffer (chilled PBS and 3% fetal bovine serum). Cells were resuspended in 300 μl FACS buffer for flow cytometry analysis using the MACSQuant VYB system. 10,000 cells per sample were counted for analysis with Flowjo v10.

### Amplicon Sequencing and Data Analysis

Amplicon sequencing, library preparation, and analysis were performed as described by Zhang (see Zhang, H. et al. Adenine base editing in vivo with a single adeno-associated virus vector. GEN Biotechnol. 1, 285-299 (2022)). Briefly, Q5 High-Fidelity polymerase (NEB #M0492) was used to amplify genomic DNA for library preparation, and libraries were pooled and purified twice after gel extraction with the Zymo gel extraction kit and DNA Clean and Concentrator (Zymo Research #11-301 and #11-303). Pooled amplicons were then sequenced on an Illumina MiniSeq system (300 cycles, Illumina sequencing kit #FC-420-1004) following the manufacturer's protocol. Sequencing data was analyzed with CRISPResso2 (see Clement, K. et al. CRISPResso2 provides accurate and rapid genome editing sequence analysis. Nat. Biotechnol. 37, 224-226 (2019)) (version 2.0.40) in BE output batch mode with and the following flags:−w 12,−wc −12,−q 30.

### Guide-Target Library Cloning

A 200-member guide-target library was designed and ordered as an oligo pool from Twist Bioscience. The oligo pool was PCR-amplified according to the recommended Twist amplification protocol. The amplified pool was then cloned via Gibson assembly into p2Tol-U6-2×BbsI-sgRNA-HygR plasmid (Addgene, #71485) cut with XbaI and BbsI. The assembled product was column-purified and electroporated into 10-beta electrocompetent cells (NEB #C3020K) as described by Miller and Arbab (see Miller, S. M. et al. Continuous evolution of SpCas9 variants compatible with non-G PAMs. Nat Biotechnol 38, 471-481 (2020); and Arbab, M. et al. Determinants of base editing outcomes from target library analysis and machine learning. Cell 182, 463-480.e30 (2020)) with the following adaptations. Following electroporation, the plasmid library was grown in an overnight liquid culture and isolated by miniprep plasmid purification. The number of transformants was assessed by serial dilution and counted colonies were above 200,000 for >1,000× library coverage.

### Guide-Target Library Cell Line Generation and Editing

Stable integration of the Tol2 guide-target library was achieved as described by Arbab (see Arbab, M. et al. Determinants of base editing outcomes from target library analysis and machine learning. Cell 182, 463-480.e30 (2020)) with the following alterations.−6×10⁶ HEK293T cells in a 10-cm plate were transfected with 30 μg plasmid DNA at a 1:1 molar ratio of Tol2 transposase plasmid to guide-target plasmid library using Lipofectamine 2000 (ThermoFisher #11668019) and following the manufacturer's protocol. 1 day post-transfection, culture media was supplemented with hygromycin [50 μg mL−1] for a minimum of 2 weeks before use in editing experiments. Library cells were maintained with over 200,000 cells for >1000× library coverage. The library cell line was transfected with ABE8e constructs that had been cloned into p2T-CMV-ABEmax-BlastR (Addgene, #152989) via Gibson assembly. For the transfections, cells were seeded with non-selective medium in 12-well plates at−200,000 cells per well and incubated overnight. The following day, cells were transfected with 1.6 μg of plasmid DNA using Lipofectamine 2000 (ThermoFisher #11668019) following the manufacturer's protocol. 1 day post-transfection, culture media was supplemented with Blasticidin S [10 μg mL-1]. After 3 days, genomic DNA was extracted from cells with QuickExtract (Lucigen #QE0905), column-purified and used for NGS library preparation.

### Guide-Target Library Editing and Analysis

NGS preparation and sequencing was done as described above with the following modifications. >1 μg of input DNA was used to ensure >500× library coverage (see Kim, H. K. et al. In vivo high-throughput profiling of CRISPR-Cpf1 activity. Nat Methods 14, 153-159 (2017)), pooled amplicons were sequenced on an Illumina NextSeq 2000 system (200 cycles, Illumina sequencing kit #20046812) following the manufacturer's protocol. Sequencing data were further processed and binned by matching spacers and their barcode sequences using a custom demultiplexing script. Sequencing data was analyzed with CRISPResso2 (version 2.0.40) in BE output batch mode. Guide-target library members with <40 reads were omitted from analysis in all samples.

### AAV Production

AAV vector packaging was done at the Viral Vector Core of the Horae Gene Therapy Center at the UMass Chan Medical School as described by Zhang (see Zhang, H. et al. Adenine base editing in vivo with a single adeno-associated virus vector. GEN Biotechnol. 1, 285-299 (2022). Constructs were packaged in AAV9 capsids and viral titers were determined by digital droplet PCR and gel electrophoresis followed by silver staining.

### Mouse Tail Vein Injection

All animal study protocols were approved by the Institutional Animal Care and Use Committee (IACUC) at UMass Chan Medical School. The 8-week-old C57BL/6 J mice (Jackson Laboratory, Stock No. 000664) were tail-vein injected with a dosage of 4×1011 vg per mouse (in 200 μl saline). Mice were euthanized at 6 weeks post injection and perfused with PBS. Livers were harvested and pulverized in liquid nitrogen, and 15 mg of the tissue from each mouse liver was used for genomic DNA extraction. Genomic DNA from mouse liver or striatum (see below) was extracted using GenElute Mammalian Genomic DNA Miniprep Kit (Millipore Sigma #G1N350). Three mice per group were used to determine in vivo editing efficiency.

### Stereotactic Intrastriatal Injection

8-15-week-old C57BL/6 J mice were weighed and anesthetized by intraperitoneal injection of a 0.1 mg/kg Fentanyl, 5 mg/kg Midazolam, and 0.25 mg/kg Dexmedetomidine mixture. Once pedal reflex ceased, mice were shaved and a total dose of 1×1010 vg of AAV was administered via bilateral intrastriatal injection (2 μL per side) performed at the following coordinates from bregma: +1.0 mm anterior-posterior (AP), ±2.0 mm mediolateral, and −3.0 mm dorsoventral. Once the injection was completed, mice were intraperitoneally injected with 0.5 mg/kg Flumazenil and 5.0 mg/kg Atipamezole and subcutaneously injected with 0.3 mg/kg Buprenorphine. Mice were euthanized at 6 weeks post-injection and perfused with PBS. Brains were harvested and biopsies at the striatum were taken for genomic DNA extraction.

### Western Blot

Plasmids encoding C-terminal 6×-His(SEQ ID NO:42) tagged Nme2-ABE8e's were delivered with sgRNA into HEK293T cells via transient transfection as described above. Protein lysates were collected 72 h post-transfection by direct addition of 2× Laemmli sample buffer (BioRad #1610737EDU) followed by lysis at 95° C. for 10 min. Western blots were performed as described by Lee (see Lee, J. et al. Tissue-restricted genome editing in vivo specified by microRNA-repressible anti-CRISPR proteins. RNA 25, 1421-1431 (2019)). Primary mouse-anti-6×His(SEQ ID NO:42) (ThermoFisher #MA1-21315, 1:2000 dilution) was used for Nme2-ABE8e detection and rabbit-anti-LaminB1 (Abcam #AB16048, 1:10,000 dilution) was used for detection of the loading control. After incubation with secondary antibodies, goat-anti-mouse IRDye®800CW (LI-COR #925-32210, 1:20,000 dilution) and goat-anti-rabbit IRDye®680RD (LI-COR #926-68071, 1:20,000 dilution), blots were visualized using a BioRad imaging system.

### Statistical Analysis

Statistical analysis was performed using one- or two-way ANOVA using Dunnett's multiple comparisons test for correction in GraphPad Prism 9.4.0.

### Example II

### Directed evolution of Nme2^{Smu}Cas9 Effectors

Nme2^{Smu}Cas9 effectors edit N₄CN PAM targets, but with reduced activity. To improve PID Chimeric Nme2 activity, compensatory mutations were introduced via rational design and directed evolution. An Nme2^{Smu}Cas9 homology model was created using the SWISS-MODEL server. Negatively charged amino acids within 5-10 angstroms of nucleic acid phosphate backbone were selected for Arginine mutagenesis (FIG. 1). Several substitutions were isolated for further analysis.

### Example III

### Nme2^{Smu}-ABE Arginine Mutations at N₄CN PAM Targets

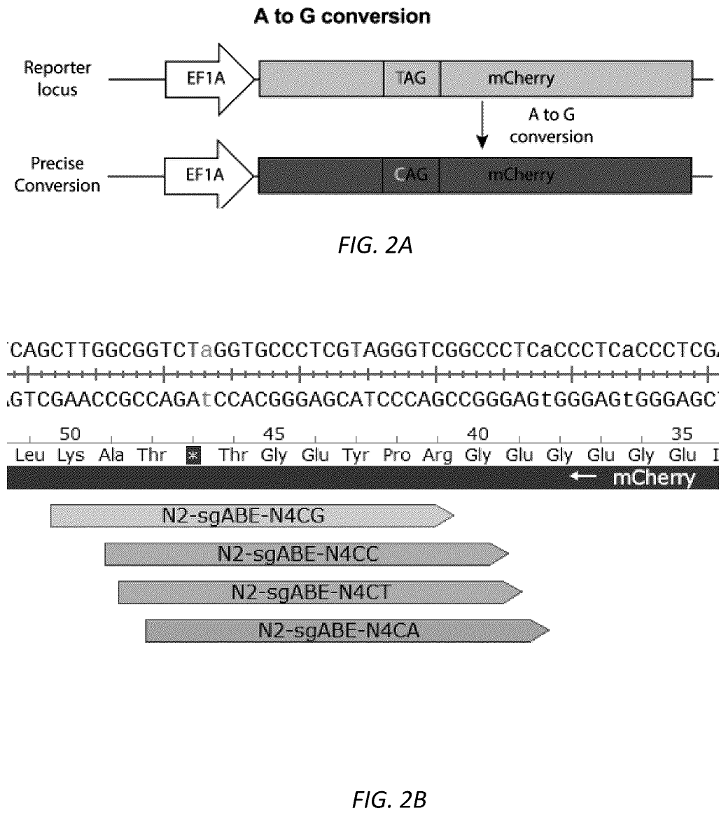

To test the efficacy of the novel Nme2^{Smu}Cas9 effectors, a modified, fluorescence-based Traffic Light Reporter (TLR2.0) was used (Certo et al., 2011). Briefly, a disrupted GFP is followed by an out-of-frame T2A peptide and mCherry cassette (FIG. 2A). When DNA double-strand breaks (DSBs) are introduced in the broken-GFP cassette, a subset of non-homologous end joining (NHEJ) repair events leave +1-frameshifted indels, placing mCherry in frame and yielding red fluorescence that can be easily quantified by flow cytometry. Homology-directed repair (HDR) outcomes can also be scored simultaneously by including a DNA donor that restores the functional GFP sequence, yielding a green fluorescence (Certo et al., 2011). Because some indels do not introduce a +1 frameshift, the fluorescence readout generally provides an underestimate of the true editing efficiency. Nonetheless, the speed, simplicity, and low cost of the assay makes it useful as an initial, semi-quantitative measure of genome editing in HEK293T cells carrying a single TLR2.0 locus incorporated via lentivector. Nme2^{Smu}Cas9 have four N₄CN PAM target sites for activating the ABE mCherry reporter (FIG. 2B).

### Example IV

### Testing Top Nme2^{Smu}-ABE Arginine Mutations at N₄CD PAM Targets

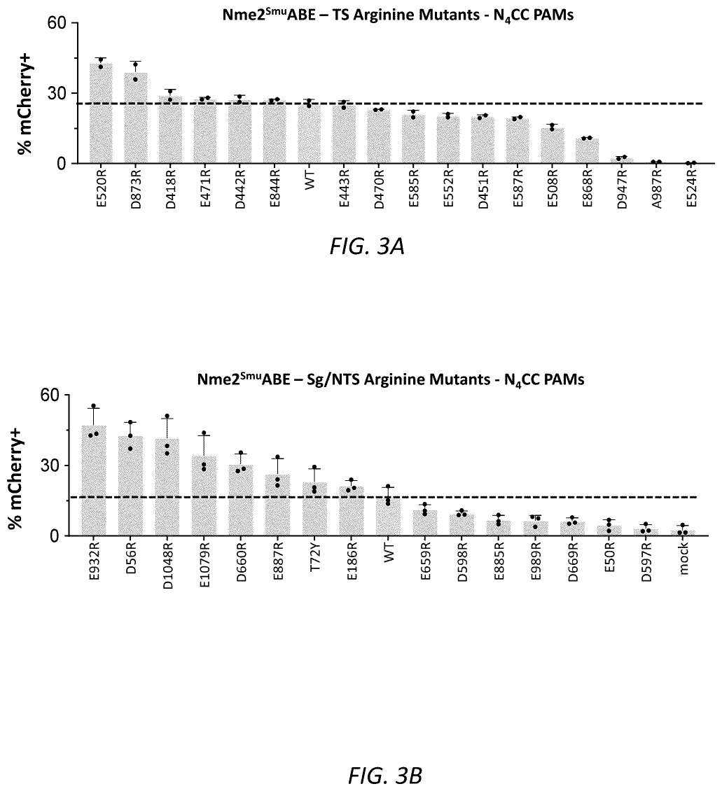

Activities of Nme2^{Smu}-ABE8e-i1 and the target-strand (TS) and of Nme2^{Smu}-ABE8e-i1, the single guide RNA (SG) and the non-target strand (NTS) interacting arginine mutants were tested in the mCherry ABE reporter cell line (activated upon A-to-G editing). Activities were measured by flow cytometry after plasmid transfection with an N₄CC PAM targeting sgRNA plasmid and a base editor plasmid (n=2 biological replicates; data represent mean±SD). Nme2^{Smu}-ABE8e-i1 variant comprising an arginine substitution at the following positions showed improved editing in the reporter assay: E520, D873, D418, E471, D442, and E844 in the Nme2^{Smu}-ABE8e-i1 and the TS (FIG. 3A) and E932, D56, D1048, E1079, D660, E887, T72, and E186 in the Nme2^{Smu}-ABE8e-i1, the SG and the NTS (FIG. 3B).

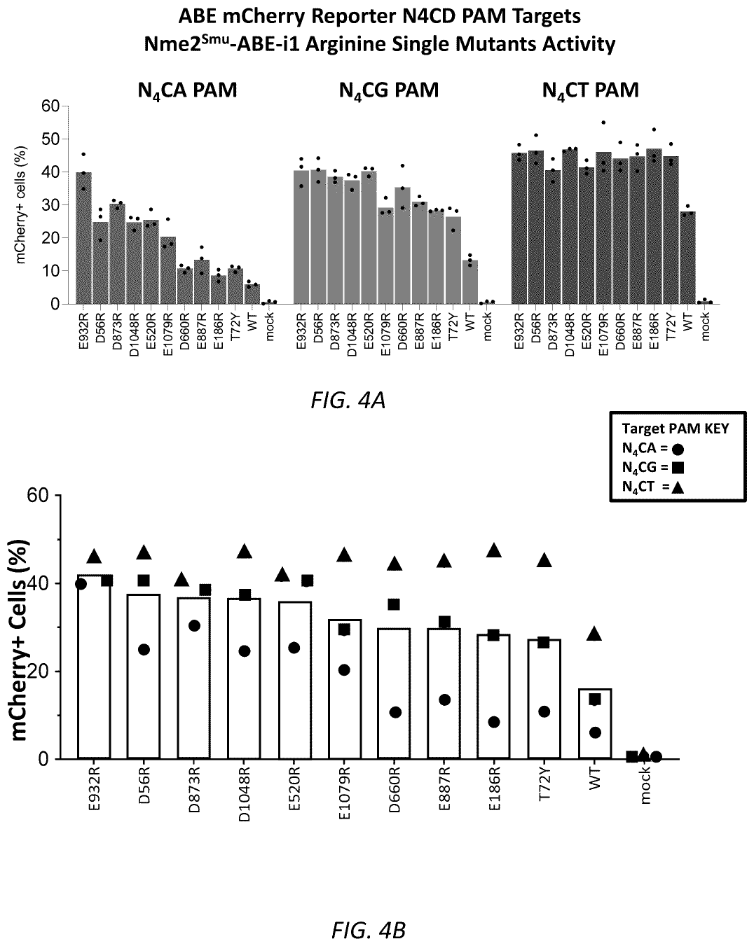

The top-performing arginine mutants (Nme2^{Smu}-ABE8e-i1 variants comprising an arginine substitution at the following positions E932, D56, D873, D1048, E520R, E1079, D660, E887, E186, and T72Y) were further tested in the mCherry ABE reporter cell line (activated upon A-to-G editing) at N₄CD PAM targets (FIG. 4A). Activities were measured by flow cytometry after plasmid transfection with associated sgRNA plasmid and a base editor plasmid (n=3 biological replicates; data represent mean±SD). The mean activity of these mutants was averaged to select for the best performing Nme2^{Smu}-ABE8e-i1 variants. All of the variants performed better than the wild type except for the variant comprising an arginine substitution at the T72Y position.

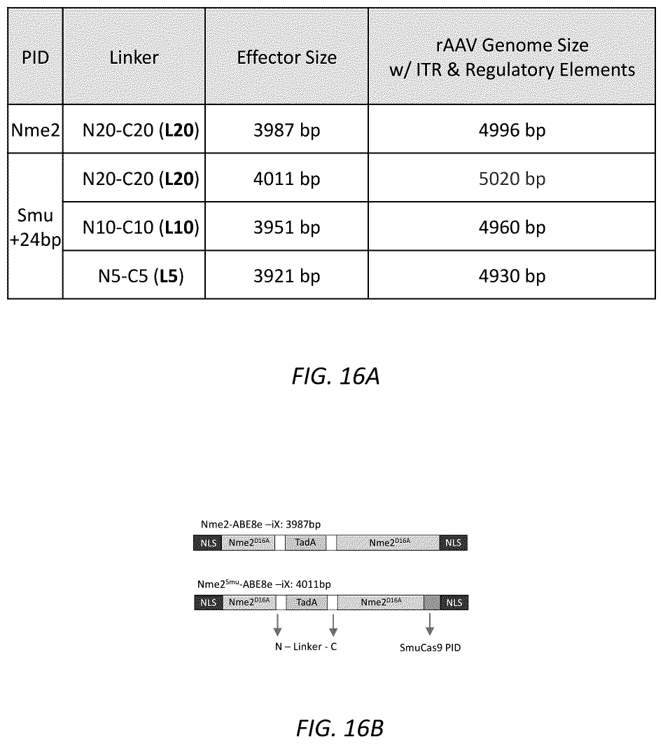

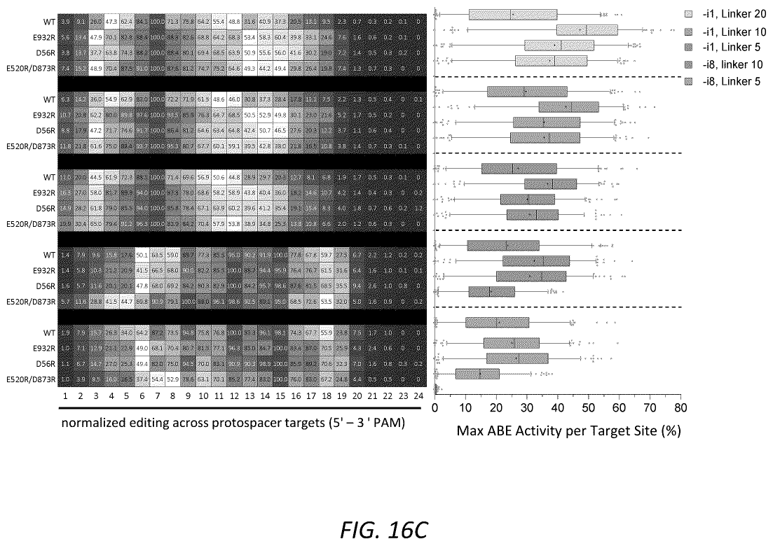

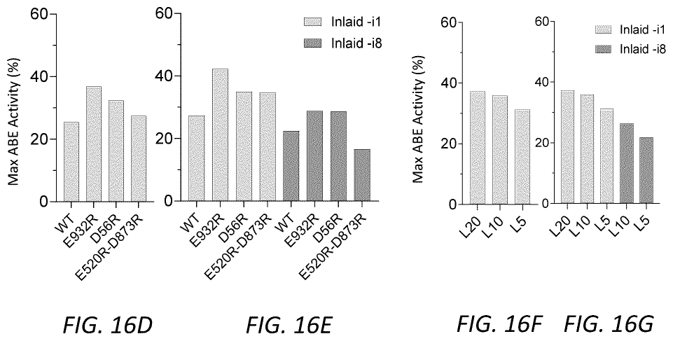

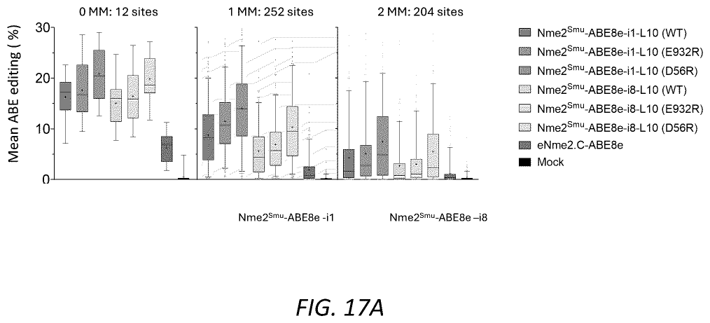

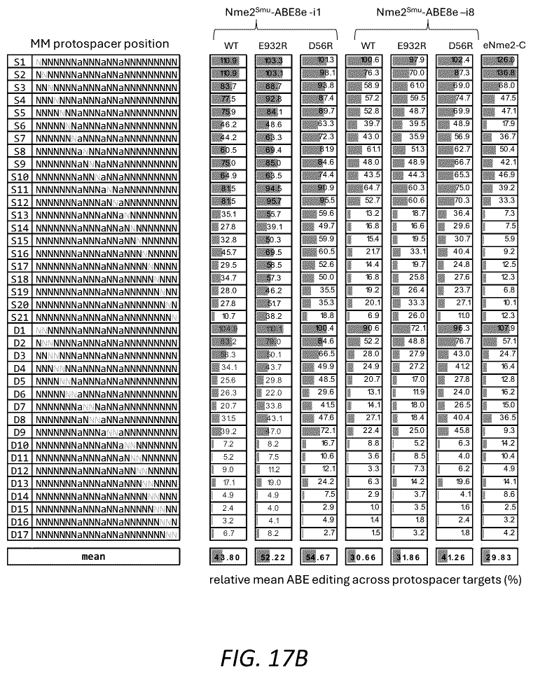

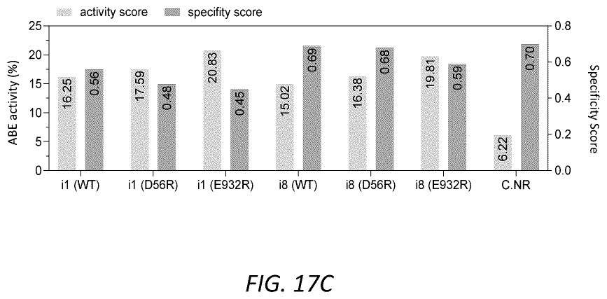

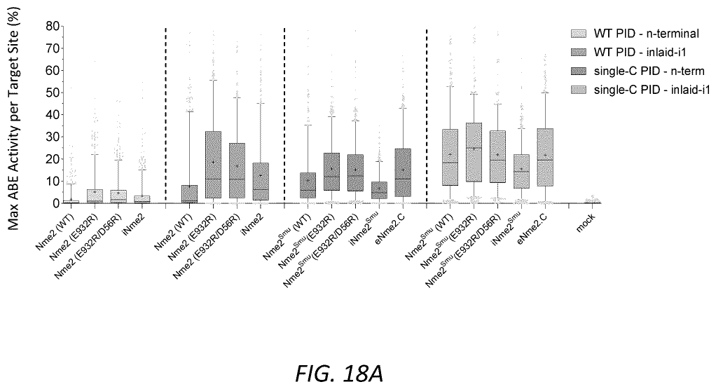

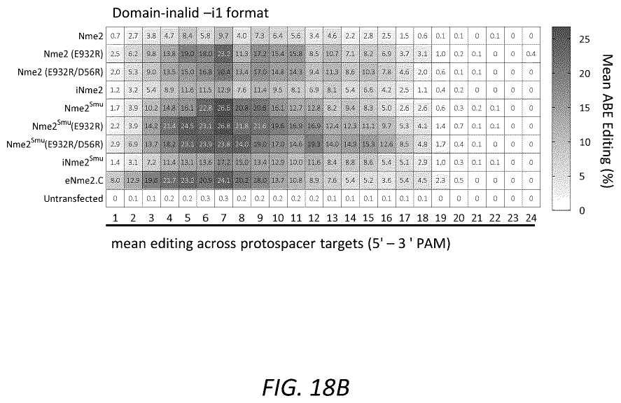

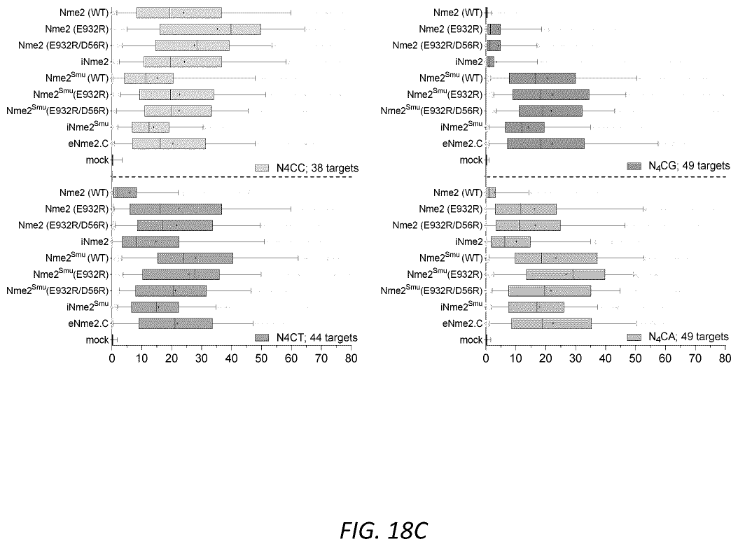

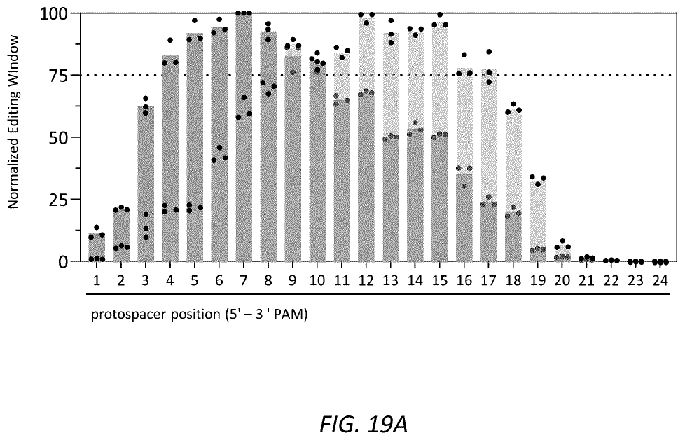

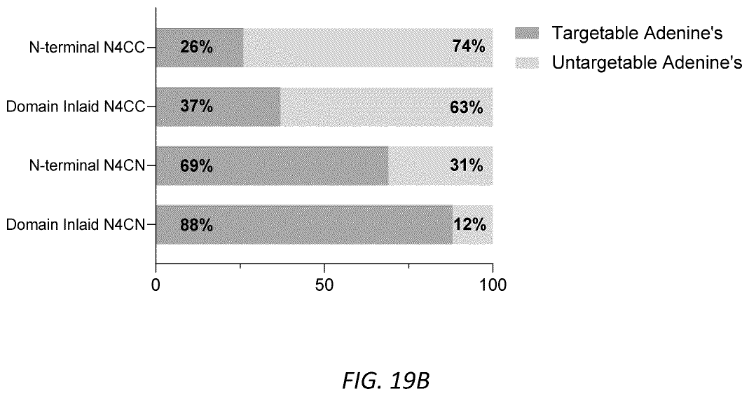

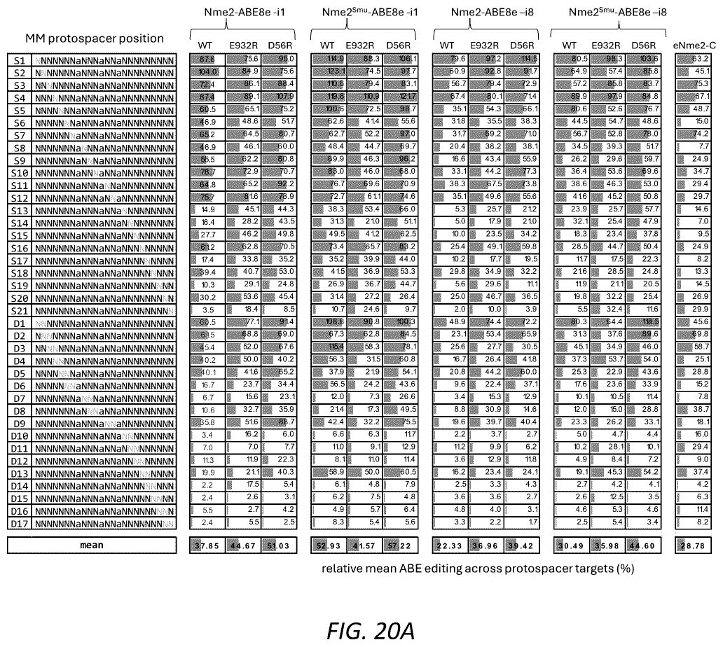

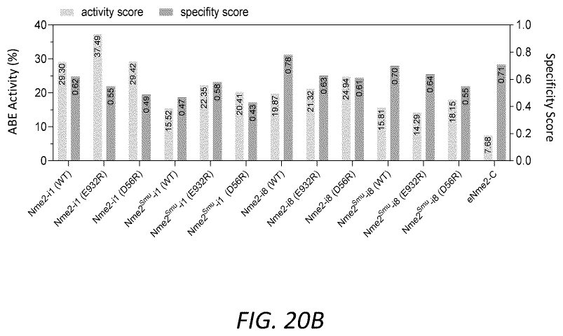

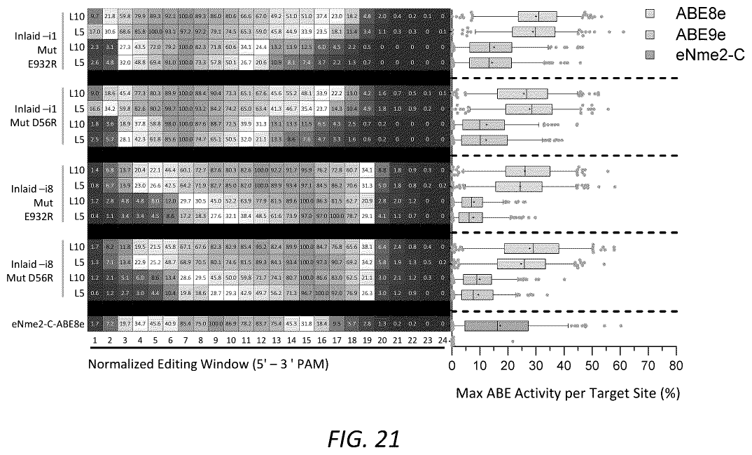

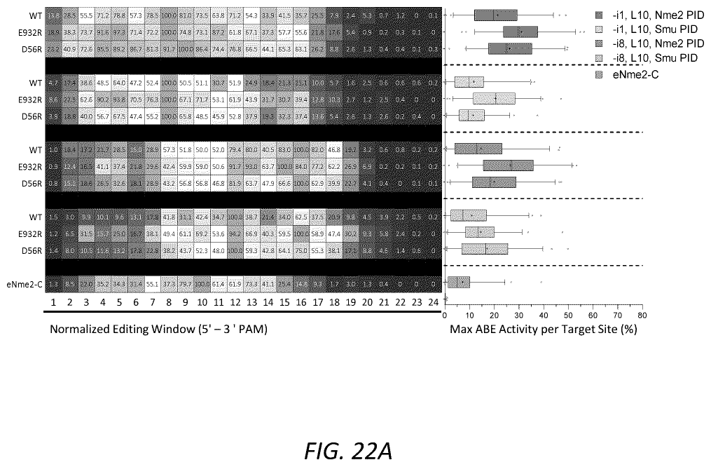

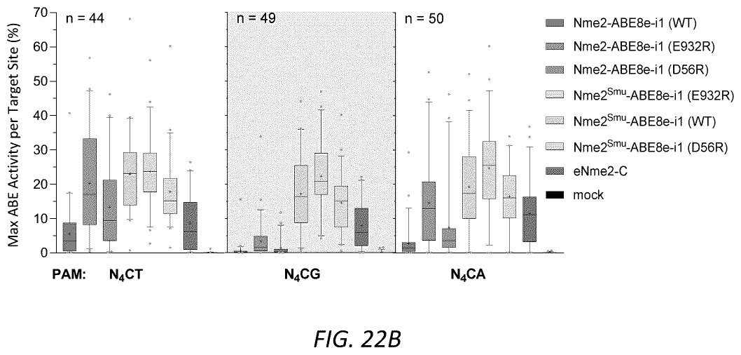

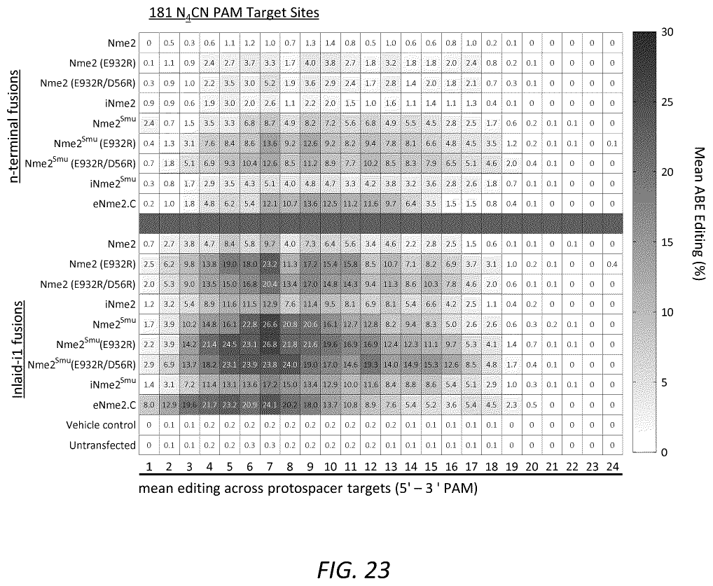

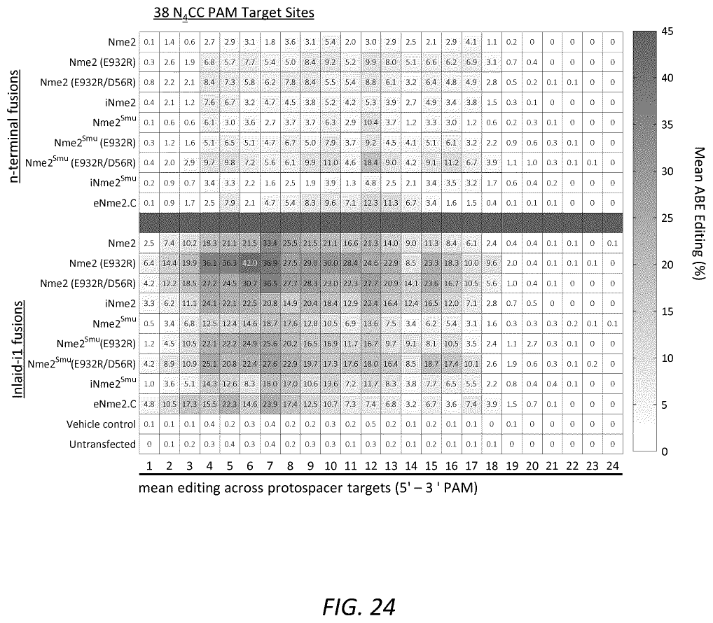

Characterization of activity of Nme2^{Smu}-ABE8e variants are also presented in FIGS. 16-24.

### Example V

### Testing Top Arginine Mutations with Nme2^{Smu}Cas9 Nuclease

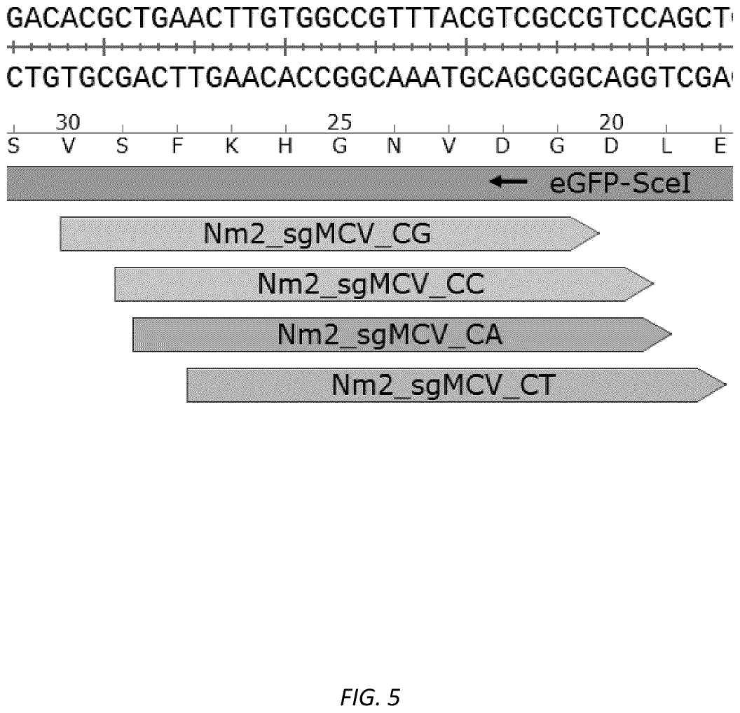

The HEK293T TLR-MCV1 reporter encodes a broken GFP, followed by a an out of frame T2A and mCherry. DSBs within a specific region of the broken GFP can result in imprecise NHEJ repair events. In cases of a +1 frameshift, mCherry is expressed. Nme2Cas9 N₄CN PAM target sites for mCherry Activation in the TLR-MCV1 reporter occurs via nuclease mediated NHEJ. Nme2Cas9 N₄CN PAM has four target sites for mCherry activation in the TLR-MCV1 reporter via nuclease mediated NHEJ (FIG. 5).

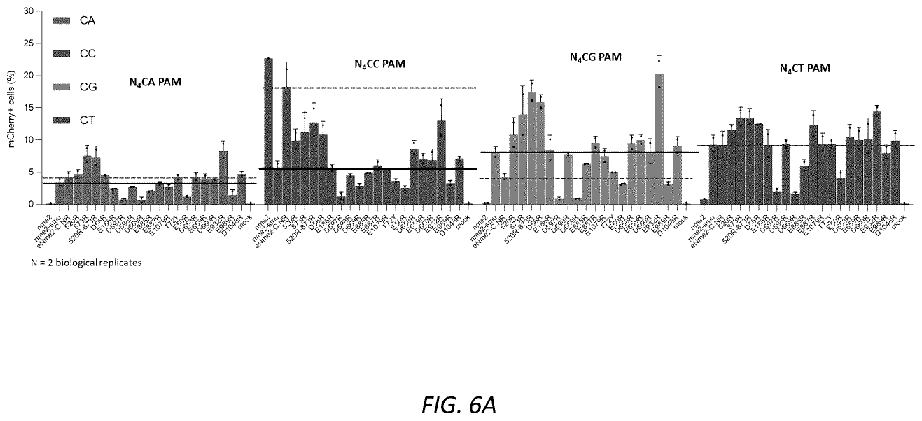

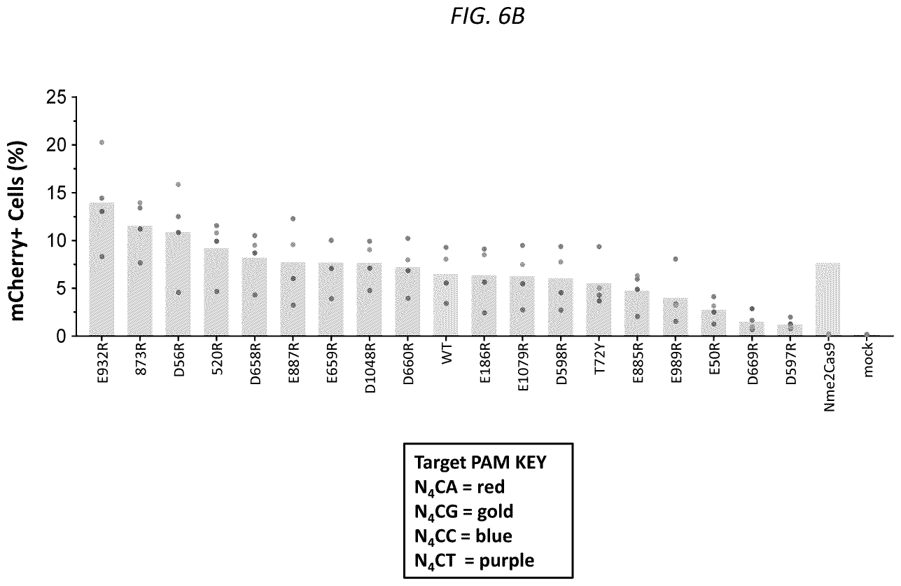

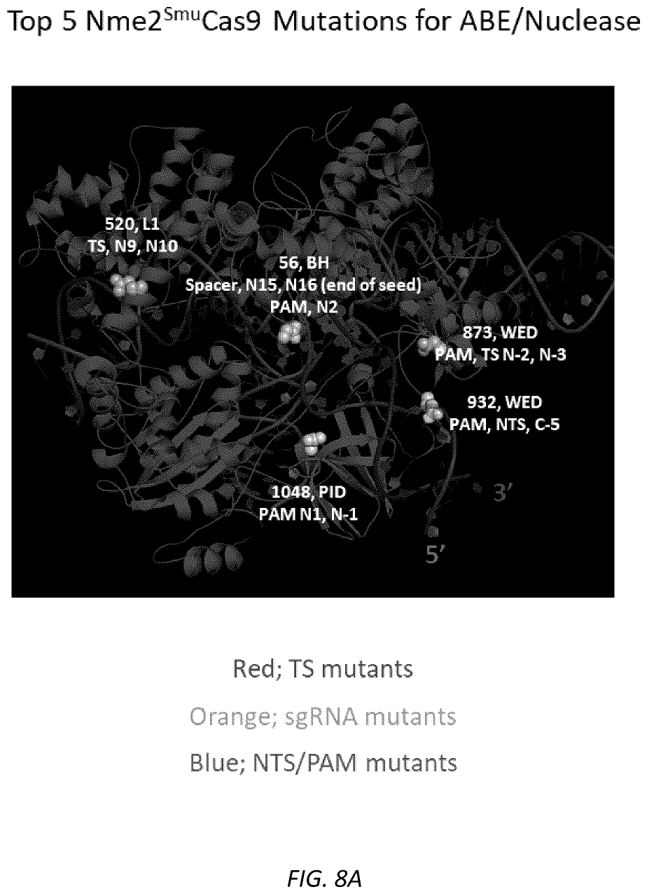

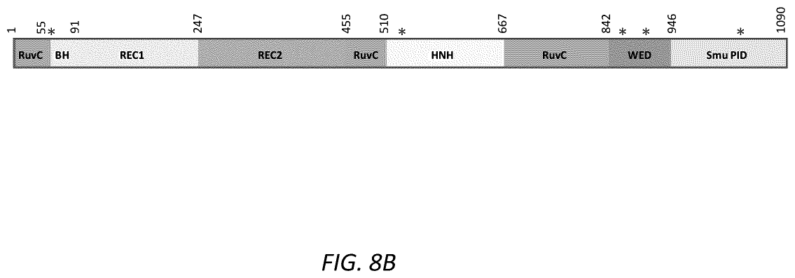

Activities of four Nme2^{Smu}Cas9 nuclease single mutants within the HEK293T TLR-MCV1 reporter were tested at N₄CN PAM targets Nme2Cas9 vs. eNme2-C·NR (vliu) vs. Nme2^{Smu}Cas9 and Nme2^{Smu}Cas9. After parallel plasmid transfection with associated sgRNA plasmid and a nuclease editor plasmid, activities were measured by flow cytometry (n=2 biological replicates; data represent mean±SD). The mean activity of these variants at a single PAM target site were then calculated to compare their performance with Nme2Cas9 and Nme2^{Smu}Cas9 as references. About half of the variants performed better than the WT (FIG. 6A and FIG. 6B), meanwhile all the variants performed better than Nme2Cas9 (FIG. 6A and FIG. 6B). The locations of the variants' top 5 activating arginine mutations can be seen in the Nme2^{Smu}Cas9 homology model built using the SWISS-MODEL server seen in FIG. 8A and FIG. 8B.

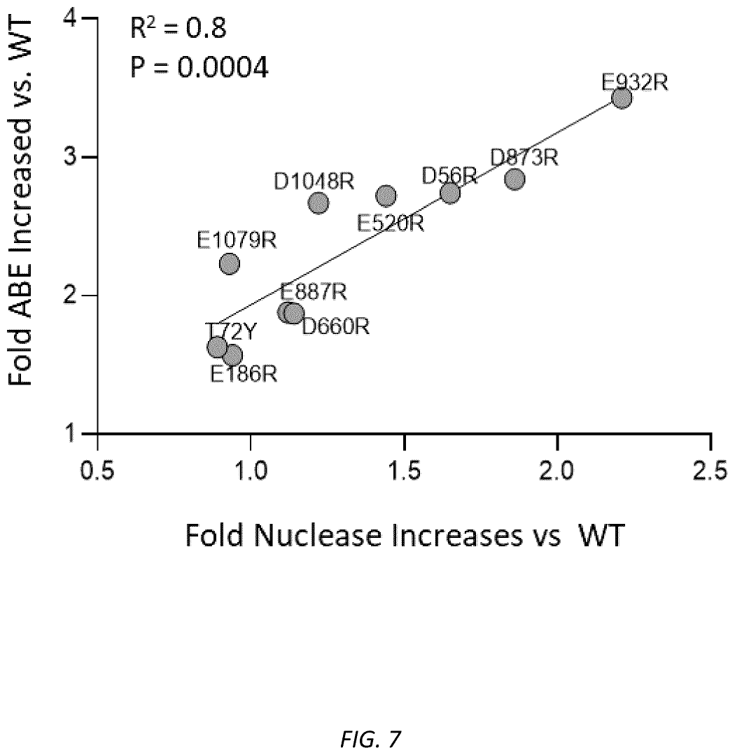

Next, to understand whether an improve in base editing activity also related to an improve in nuclease activity, the correlation between ABE and nuclease Nme2^{Smu}Cas9 effectors was measured. Indeed, the observed activity of the top performing Nme2^{Smu} Arginine mutations correlate for nuclease and ABE editing when compared to Wild-Type Nme2^{Smu}Cas9 (nuclease) or Nme2^{Smu}-ABE8e-i1 (ABE) in the reporter assays (FIG. 7).

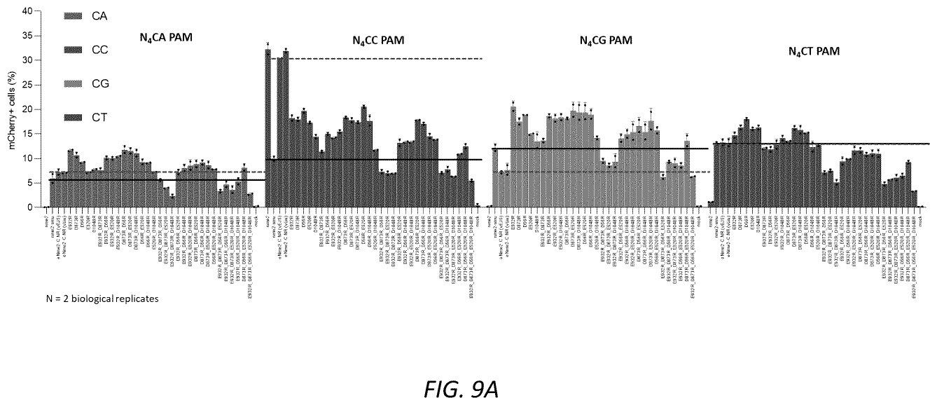

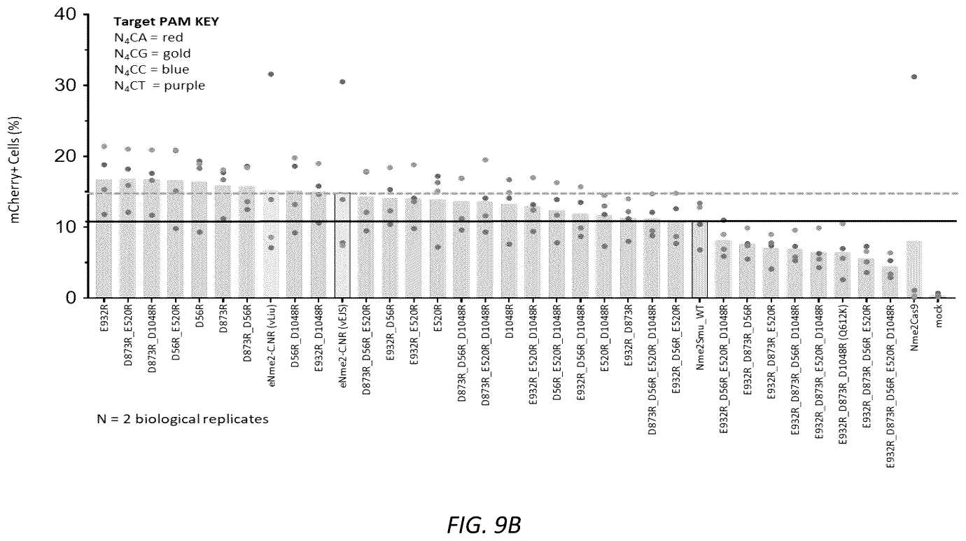

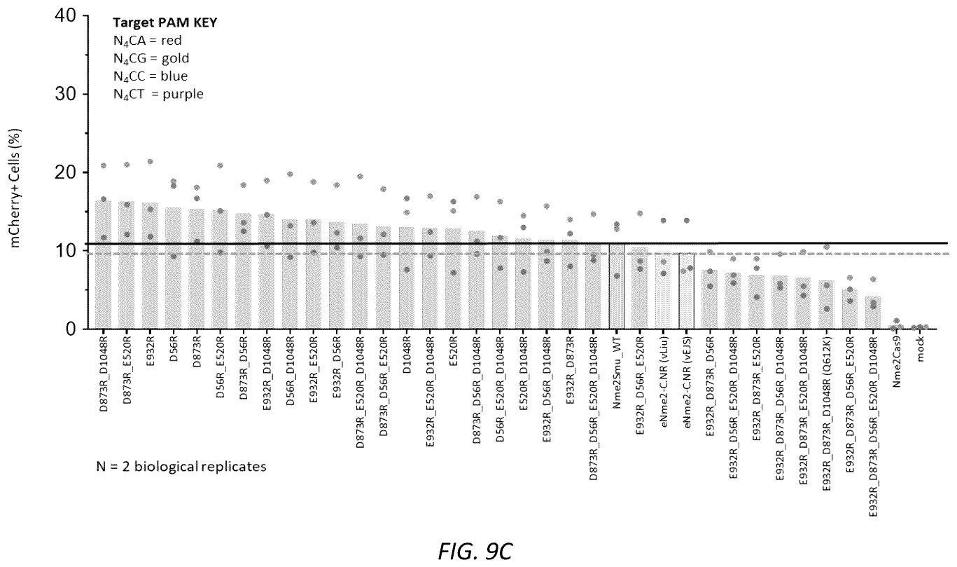

The activities of the nuclease variants were also tested for combination mutants within the HEK293T TLR-MCV1 reporter at N₄CN PAM targets. Nme2Cas9, eNme2-C·NR (vliu), eNme2-C·NR (vEJS), and Nme2^{Smu}Cas9 and Nme2^{Smu}Cas9's nuclease activity was tested at N₄CA, N₄CC, and N₄CG PAM targets (FIG. 9A). After parallel plasmid transfection with associated sgRNA plasmid and a nuclease editor plasmid, activities were measured by flow cytometry (n=2 biological replicates; data represent mean±SD). The average activity for the Nme2^{Smu} mutants is increased compared to eNme2-C·NR at N₄CD PAM target sites for activating the TLR-MCV1 reporter (FIGS. 9B and 9C).

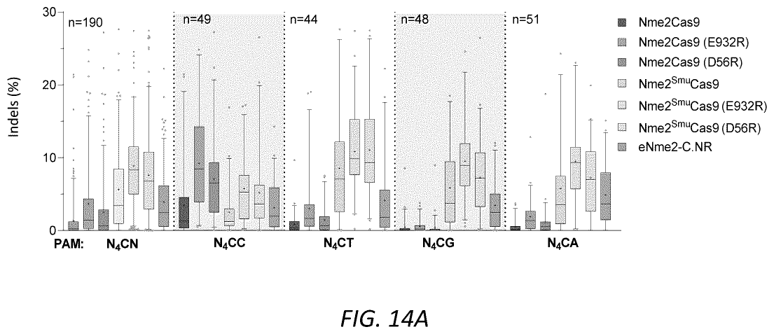

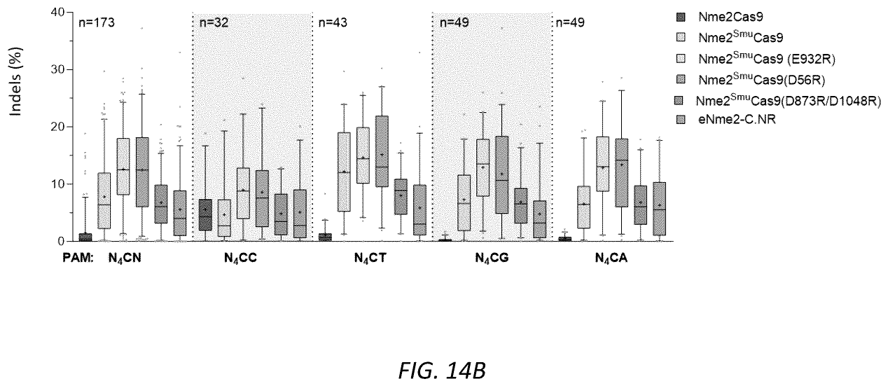

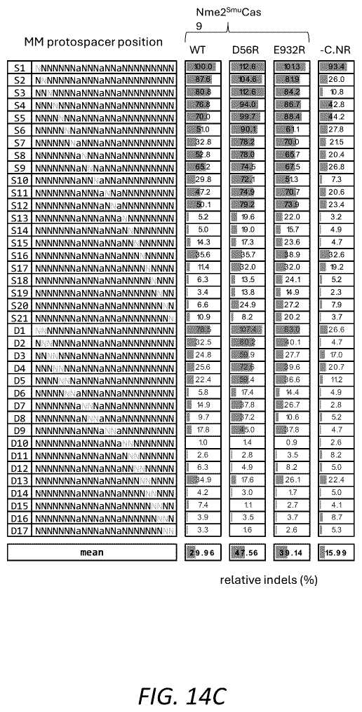

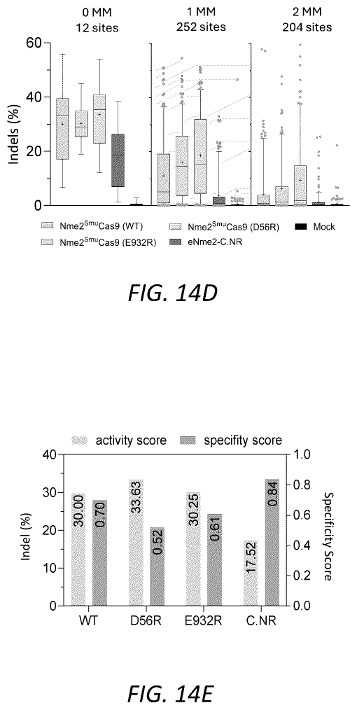

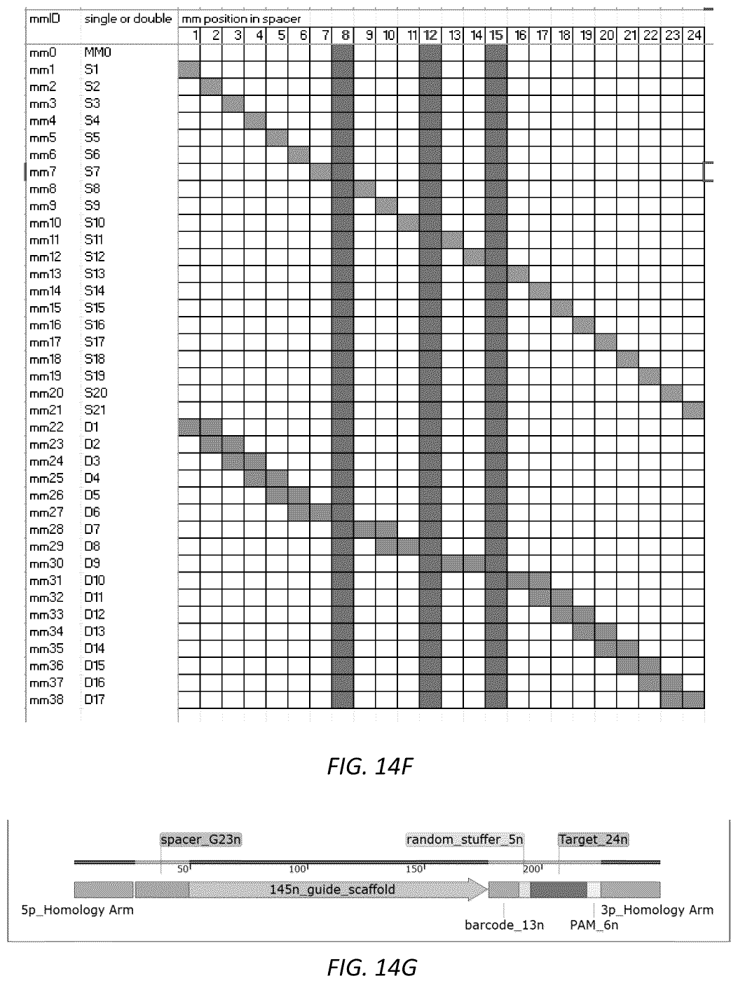

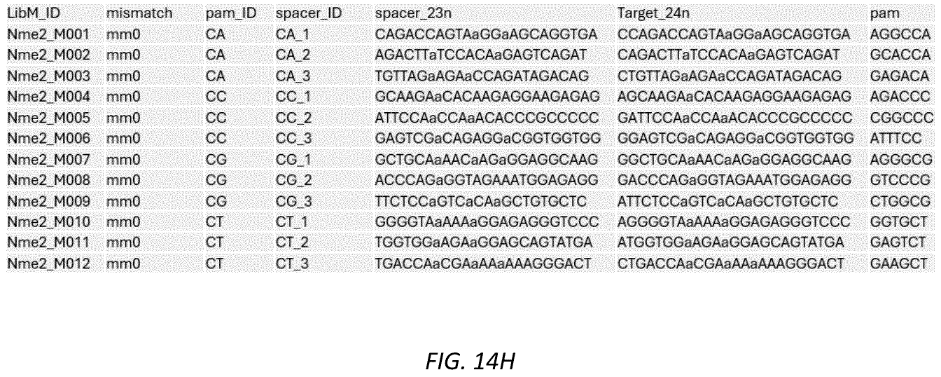

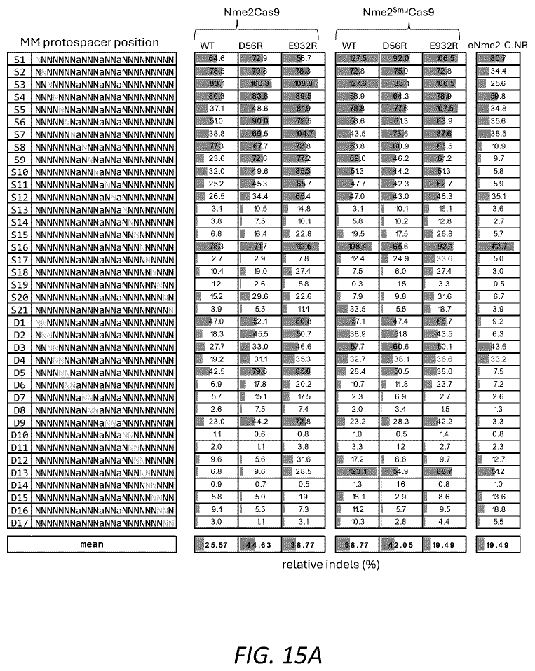

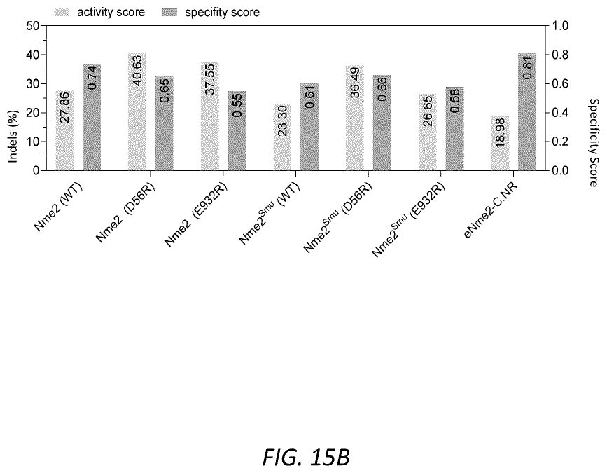

Characterization of the activity and specificity of Nme2- and Nme2^{Smu}Cas9 nuclease variants are also presented in FIG. 14 and FIG. 15.

### Example VI

### Compatibility of Enhanced Arginine Mutations with Nme2Cas9 ABE and Nuclease at N₄CC PAM Targets

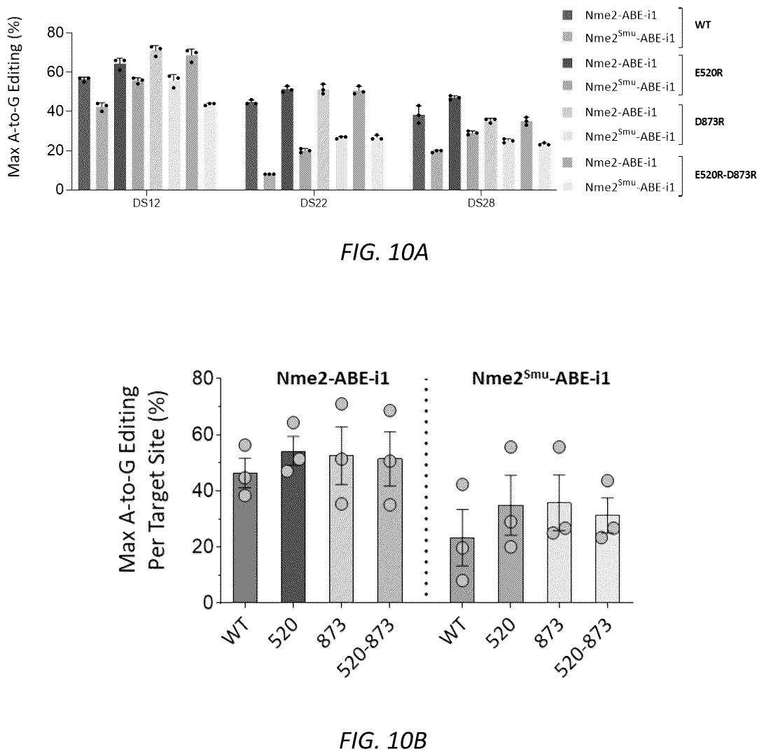

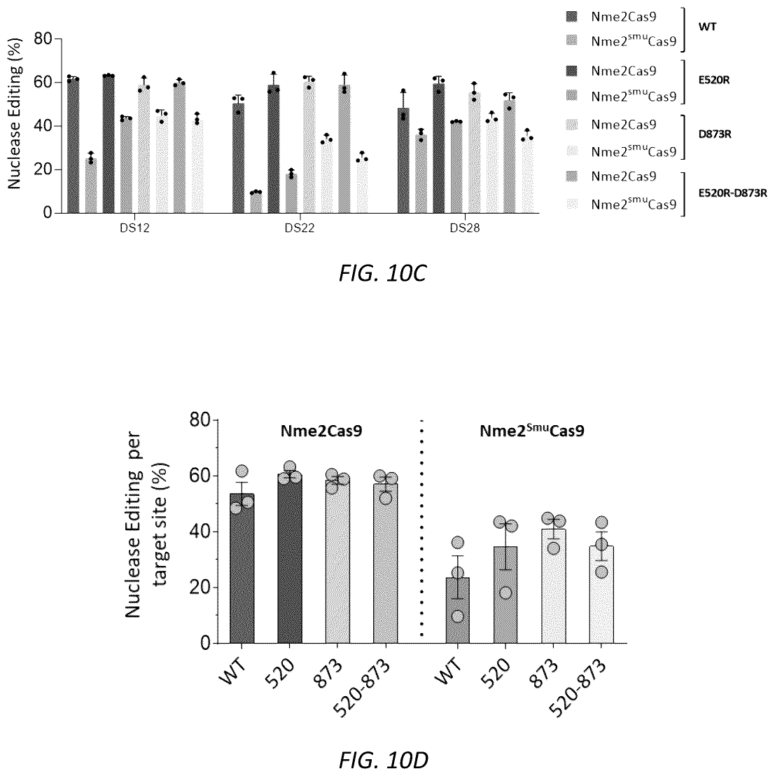

A-to-G edits were performed at endogenous HEK293T genomic loci with Nme2-ABE81-i1 or Nme2^{smu}-ABE8e-i1 constructs by plasmid transfection to test the adenine edits for each target. Maximum A-to-G editing rates (FIG. 10A), maximum A-to-G editing rates per site (FIG. 10B), percent nuclease editing (FIG. 10C), and percent nuclease editing per site (FIG. 10D) of the WT, E520R, D873R, and E520R-D873R constructs at each individual N₄CC target site were measured. Base and nuclease editing efficiencies of the arginine mutants are higher than that of the WT for both the Nme2-ABE81-i1 and the Nme2^{smu}-ABE8e-i1 constructs (FIGS. 10A, 10B, 10C, and 10D).

### Example VII

### Optimizing Size of Domain-Inlaid Nme2^{Smu}-ABE's for AAV Compatibility

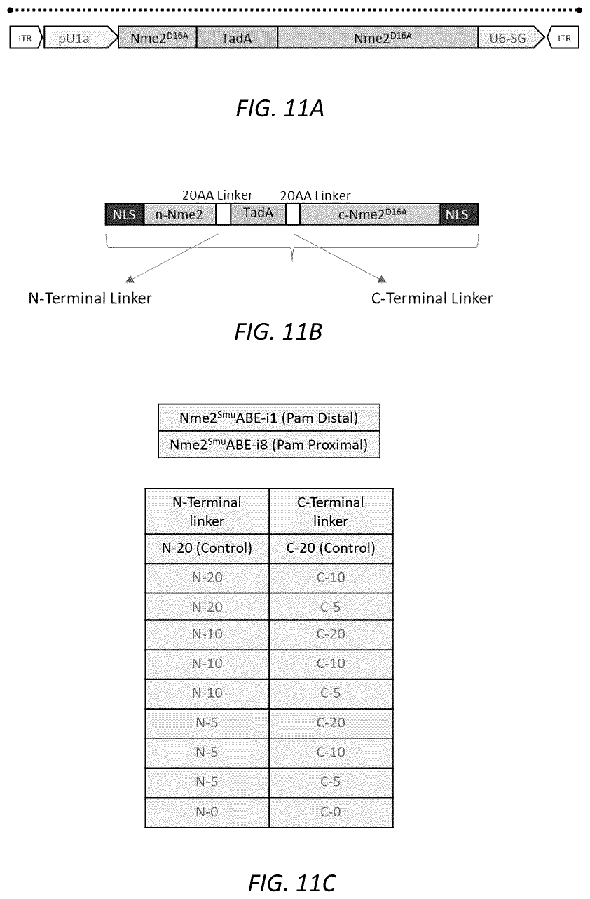

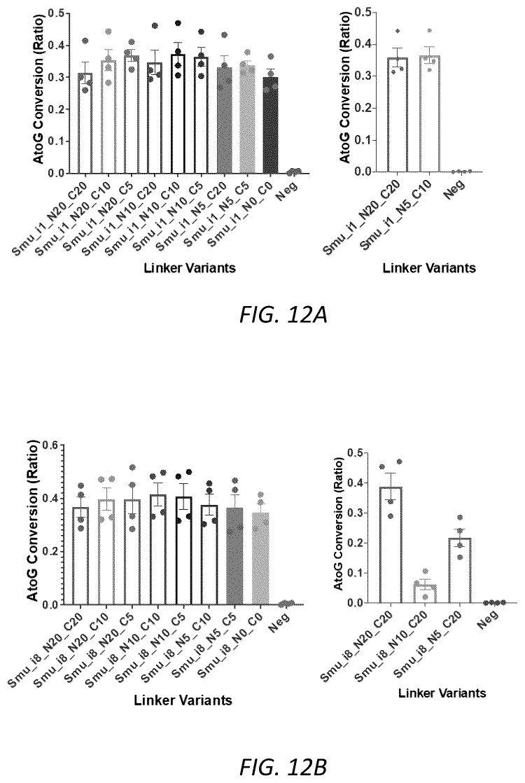

The Nme2Cas9 all-in-one AAV delivery platform, can in principle, be used to target as wide a range of sites (FIG. 11A). Domain-inlaid Nme2Cas9 nucleotide base editors were previously designed and showed improved editing efficiencies and improved modulation of editing windows. These editors possessed linker flanked TadA8e that were inserted into these internal sites (FIG. 11B). The original Nme2^{Smu}-ABE-i1 transgene has 20 amino acid linkers flanking each side of deaminase (N-term linker, N-20) and (C-term linker, C-20). A combination of N-terminal and C-Terminal linkers flanking the TadA8e deaminase domain for size minimized Nme2^{Smu}-ABE-i1 transgenes were tested for the arginine-enhanced constructs (FIG. 11C). These combinations of new N- and C-linkers flanking the TadA8e deaminase in the Nme2^{Smu}-ABE-i1 transgene were all active at the target sites tested and were size-compatible for recombinant AAV packaging (FIGS. 12A and 12B).

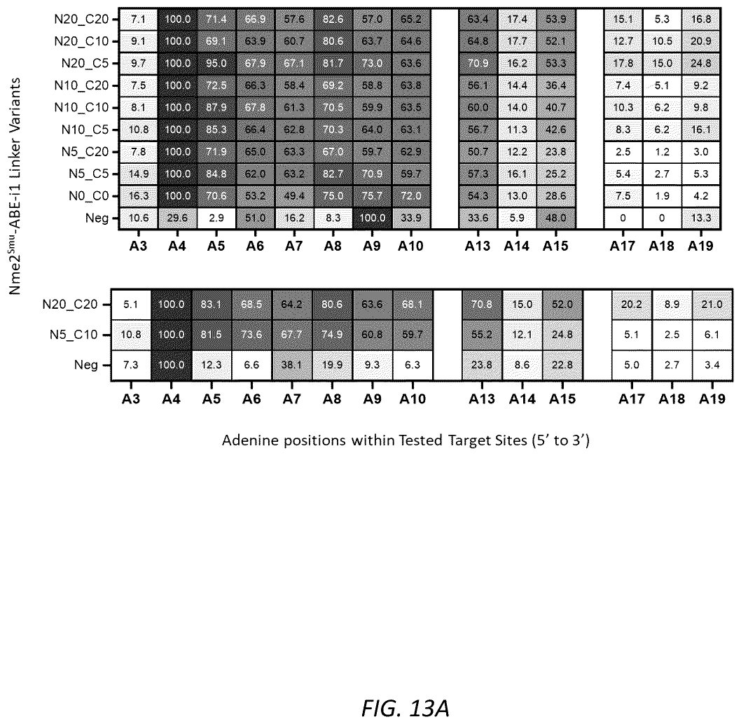

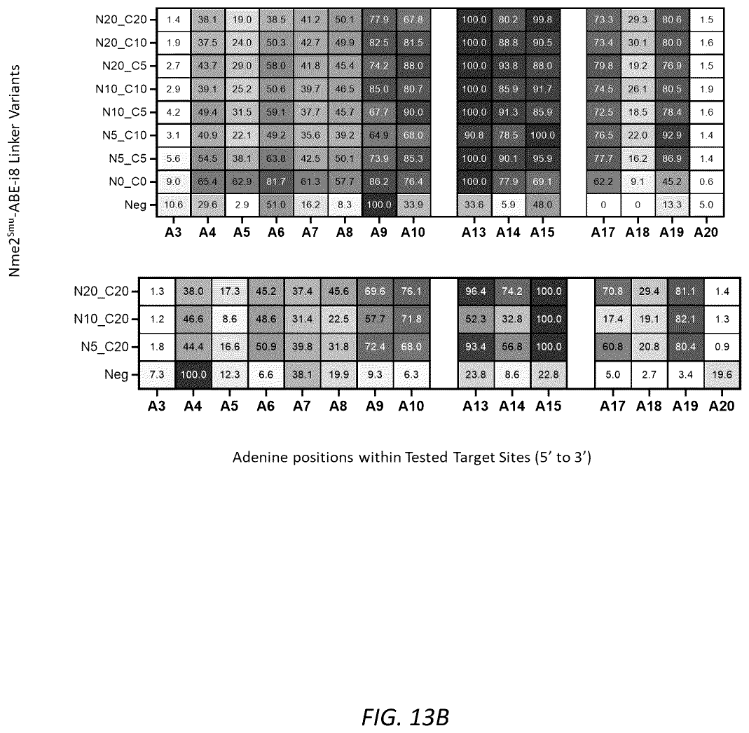

The editing windows of Nme2Smu-ABE-i1 (FIG. 13A) and Nme2Smu-ABE-i8 (FIG. 13B) linker variants were further tested at four endogenous N₄CN PAM Targets in HEK293T. The A-to-G conversion for each variant showed that adenine position A4 (5′ to 3′) within target site showed the highest observed edited efficiencies for Nme2Smu-ABE-i1 and position A13 (5′ to 3′) within target site showed the highest observed edited efficiencies for Nme2^{Smu}-ABE-i8.

### Example VIII

### Analysis of Domain-Inlaid Nme2-ABE8e Specificity

The specificities of the domain-inlaid Nme2-ABEs were determined. Guide-dependent off-target editing is driven by Cas9 unwinding and R-loop formation at targets with high sequence similarity. Nme2-ABE8e-nt has a much lower propensity for guide-dependent off-target editing compared to Spy-ABE8e. Using the most active inlaid variant (Nme2-ABE8e-i1) as a prototype, guide-dependent specificity was examined using a series of double-mismatch guides targeting the mCherry reporter, with Spy-ABE8e and Nme2-ABE8e-nt used for comparison. In all cases, the target adenosine was at the eighth nt of the protospacer (FIG. 26A, FIG. 26B). To account for differences in on-target activity (especially for Nme2-ABE8e-nt), the activities of the mismatched guides were normalized to that of the respective non-mismatched guide. It was found that Spy-ABE8e significantly outperformed Nme2-ABE8e-nt for on-target activity (FIG. 26A), but exhibited far greater activity with mismatched guides (FIG. 26B). Nme2-ABE8e-i1 activated the reporter with a similar efficiency as Spy-ABE8e (FIG. 26B), but with greater sensitivity to mismatches (FIG. 26B). Although the Nme2-ABE8e-i1 variant was less promiscuous than Spy-ABE8e, it exhibited higher activity with mismatched guides than Nme2-ABE8e-nt, illustrating trade-offs between on- and off-target editing efficiencies. The mismatch sensitivity of the Nme2-ABE8e−i7 and −i8 effectors were then assayed to determine whether their preference for PAM-proximal editing windows would alter the mismatch sensitivity in comparison to the−nt and −i1 effectors for activating the reporter cell line. In this experiment, Nme2-ABE8e-i7 and −i8 exhibited mismatch sensitivities comparable to Nme2-ABE-nt, while retaining high on-target activity (FIG. 26A, FIG. 26B). A potential explanation for the increased sensitivity of−i7 and −i8 effectors at this site is that the impact of imperfect base pairing between a guide and target may become more apparent as the optimal editing window shifts away from the target adenine.

The specificity of domain-inlaid Nme2- and Nme2^{Smu}-ABE8e's against their respective ABE8e-nt variants at bona fide endogenous off-target sites was then evaluated. Although Nme2Cas9 off-target sites are rare due to its intrinsic accuracy in mammalian genome editing, a few off-target sites have been identified for both nuclease and ABE variants via GUIDE-seq or in silico prediction. Four target sites for assessment were selected, of which three had been validated as detectably edited off-target sites (see Zhang, H. et al. Adenine base editing in vivo with a single adeno-associated virus vector. GEN Biotechnol. 1, 285-299 (2022); Edraki, A. et al. A compact, high-accuracy Cas9 with a dinucleotide PAM for in vivo genome editing. Molecular Cell 73, 714-726.e4 (2019); and Huang, T. P. et al. High-throughput continuous evolution of compact Cas9 variants targeting single-nucleotide-pyrimidine PAMs. Nat Biotechnol (2022)) (FIG. 26C). In agreement with the mismatch sensitivity assay, Nme2-ABE8e variants with domain insertion at the−i1 position exhibited the greatest increase in off-target editing efficiencies, reaching above 1% at two out of the four targets and yielding the least favorable specificity ratio [on-target:off-target editing ratio] of−23:1. Also in agreement with the mismatch sensitivity assay, the−i7 and −i8 effectors displayed increased accuracy in comparison to the−nt effectors (with specificity ratios of ˜200:1 for −i7, ˜170:1 for −i8, and ˜82:1 for −nt) (FIG. 26C).

Guide-independent off-target editing were then assessed. Similar to other domain-inlaid BE architectures, the internal positioning of the deaminase was expected to limit the propensity for off-target nucleic acid editing that occurs in trans. The orthogonal R-loop assay with HNH-nicking SauCas9 (nSau^{D10A}) (see Richter, M. F. et al. Phage-assisted evolution of an adenine base editor with improved Cas domain compatibility and activity. Nat Biotechnol. (2020); Chu, S. H. et al. Rationally designed base editors for precise editing of the sickle cell disease mutation. The CRISPR Journal 4, 169˜177 (2021); and Doman, J. L., Raguram, A., Newby, G. A. & Liu, D. R. Evaluation and minimization of Cas9-independent off-target DNA editing by cytosine base editors. Nat Biotechnol 38, 620˜628 (2020)) was used to generate off-target R-loops and capture the guide-independent DNA editing mediated by Spy-ABE8e or the Nme2-ABE8e variants (−nt- and i1). The on- and off-target activity of these ABE8e effectors was evaluated by amplicon deep sequencing at the guide-targeted genomic site in addition to three SauCas9^{D10A}_generated R-loops. Nme2-ABE8e-i1 was found to be less prone to editing the orthogonal R-loops compared to Nme2-ABE8e-nt and Spy-ABE8e (FIG. 27A). To account for differences in on-target activities of the effectors, the data were reanalyzed by assessing the on-target: off-target editing ratio of each effector. Since Nme2-ABE8e effectors (-nt and −i1) have wider editing windows than Spy-ABE8e, the average editing activities across the respective windows of each effector for this target was used (protospacer positions 1˜17nt for Nme2-ABE8e and 3˜9nt for Spy-ABE8e), enabling a better comparison between the effectors (FIG. 27B). In all cases, Nme2-ABE8e-i1 was found to significantly outperformed Nme2-ABE8e-nt and Spy-ABE8e for guide-independent specificity at the orthogonal R-loops tested (FIG. 27C). For this assay, whether the TadA8e^{V106W} mutant further increases the guide-independent DNA specificity with the Nme2-ABE8e-i1 architecture was also investigated (Nme2-ABE8e^{v106w}-i1). It should be noted that V106 corresponds to the TadA8e amino acids sequence that contains a N terminal methionine. In a TadA8e sequence without the N terminal methionine, such as used in the Nme2-ABE8e^{v106w}-i1 polypeptide, the V106W substitution is at position 105. Increased specificity at all orthogonal R-loops with Nme2-ABE8e^{v106w}-i1 compared to Nme2-ABE8e-i1 was observed, though the specificity increase was only significant for R-loop 3 (SSH2) (FIG. 27C).

### Example IX

### Domain-inlaid Nme2-ABE8e Enables In Vivo Base Editing With a Single AAV Vector

A compact AAV design that enables all-in-one delivery of Nme2-ABE8e-nt with a sgRNA for in vivo base editing was used (see Zhang, H. et al. Adenine base editing in vivo with a single adeno-associated virus vector. GEN Biotechnol. 1, 285˜299 (2022)). At 4996 bp, the cassettes harboring the domain-inlaid Nme2-ABE8e variants and a guide RNA are also within the packaging limit of some single AAV vectors, allowing to test whether they outperform Nme2-ABE8e-nt in an in vivo setting. For the in vivo assay, AAV genomes containing Nme2-ABE8e-nt, Nme2-ABE8e-i1 or Nme2-ABE8evio^{6w}-i1 with an sgRNA targeting the Rosa26 locus were designed (FIG. 28A).

Tow in vivo editing experiments with 9-week-old mice were conducted. First, systemic [intravenous (i.v.)] injection and editing in the liver as assessed, whereas the second experiment tested editing in the brain after intrastriatal injection. In both cases, mice were sacrificed 6 weeks after their respective injections and editing was quantified by amplicon sequencing. Within the liver, Nme2-ABE8e-i1 and Nme2-ABE-i1^{V106W} had editing efficiencies of ˜49% (p=0.015) and ˜46% (p=0.04) respectively, outperforming Nme2-ABE8e-nt (editing efficiency ˜34% at A6 of the Rosa26 target site), (one-way ANOVA) (FIG. 28B). Within the striatum the trend continued, with both Nme2-ABE8e-i1 and Nme2-ABE-i1^{V106W} exhibiting improved editing activities (˜37% and ˜34% at A6 of Rosa26), compared to Nme2-ABE-nt (˜25%), albeit this improvement did not reach statistical significance (p=0.26 and 0.5, for Nme2-ABE8e-i1 and Nme2-ABE-i1^{V106W} respectively) (FIG. 28B).

Whether the boost in on-target activity in the liver was also accompanied by increased sgRNA-dependent off-target activity was then determined. The Rosa26 sgRNA used in this assay is unusual among Nme2Cas9 guides in having a previously validated off-target site (Rosa26-OT1). Amplicon sequencing at Rosa26-OT1 on genomic DNA extracted from the mouse livers was conducted. It was found that both Nme2-ABE8e-i1 and the V106W variant increased off-target A-to-G editing (up to ˜7% and ˜5% respectively) compared to Nme2-ABE8e-nt (˜0.2%) (FIG. 28C). These results demonstrate that the increased activity of the domain-inlaid ABEs can translate to an in vivo setting.

### REFERENCES

The contents of all cited references (including literature references, patents, patent applications, patent publications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Amrani, N., Gao, X. D., Liu, P., Edraki, A., Mir, A., Ibraheim, R., Gupta, A., Sasaki, K. E., Wu, T., Donohoue, P. D., et al. (2018). NmeCas9 is an intrinsically high-fidelity genome editing platform. BioRxiv, doi.org/10.1101/172650.
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709˜1712.
Bisaria, N., Jarmoskaite, I., and Herschlag, D. (2017). Lessons from Enzyme Kinetics Reveal Specificity Principles for RNA-Guided Nucleases in RNA Interference and CRISPR-Based Genome Editing. Cell Syst. 4, 21˜29.
Bolukbasi, M. F., Gupta, A., Oikemus, S., Derr, A. G., Garber, M., Brodsky, M. H., Zhu, L. J., and Wolfe, S. A. (2015a). DNA-binding-domain fusions enhance the targeting range and precision of Cas9. Nat. Methods 12, 1150˜1156.
Bolukbasi, M. F., Gupta, A., and Wolfe, S. A. (2015b). Creating and evaluating accurate CRISPR-Cas9 scalpels for genomic surgery. Nat. Methods 13, 41˜50.
Brinkman, E. K., Chen, T., Amendola, M., and van Steensel, B. (2014). Easy quantitative assessment of genome editing by sequence trace decomposition. Nucleic Acids Res. 42, e168.
Brouns, S. J., Jore, M. M., Lundgren, M., Westra, E. R., Slijkhuis, R. J., Snijders, A. P., Dickman, M. J., Makarova, K. S., Koonin, E. V., and van der Oost, J. (2008). Small CRISPR RNAs guide antiviral defense in prokaryotes. Science 321, 960˜964.
Casini, A., Olivieri, M., Petris, G., Montagna, C., Reginato, G., Maule, G., Lorenzin, F., Prandi, D., Romanel, A., Demichelis, F., et al. (2018). A highly specific SpCas9 variant is identified by in vivo screening in yeast. Nat. Biotechnol. 36, 265˜271.
Certo, M. T., Ryu, B. Y., Annis, J. E., Garibov, M., Jarjour, J., Rawlings, D. J., and Scharenberg, A. M. (2011). Tracking genome engineering outcome at individual DNA breakpoints. Nat. Methods 8, 671˜676.
Chen, J. S., Dagdas, Y. S., Kleinstiver, B. P., Welch, M. M., Sousa, A. A., Harrington, L. B., Sternberg, S. H., Joung, J. K., Yildiz, A., and Doudna, J. A. (2017). Enhanced proofreading governs CRISPR-Cas9 targeting accuracy. Nature 550, 407˜410.
Cho, S. W., Kim, S., Kim, J. M., and Kim, J. S. (2013). Targeted genome engineering in human cells with the Cas9 RNA-guided endonuclease. Nat. Biotechnol. 31, 230˜232.
Cho, S. W., Kim, S., Kim, Y., Kweon, J., Kim, H. S., Bae, S., and Kim, J. S. (2014). Analysis of off-target effects of CRISPR/Cas-derived RNA-guided endonucleases and nickases. Genome Res. 24, 132˜141.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819˜823.
Deltcheva, E., Chylinski, K., Sharma, C. M., Gonzales, K., Chao, Y., Pirzada, Z. A., Eckert, M. R., Vogel, J., and Charpentier, E. (2011). CRISPR RNA maturation by trans-encoded small RNA and host factor RNase III. Nature 471, 602˜607.
Deveau, H., Barrangou, R., Garneau, J. E., Labonte, J., Fremaux, C., Boyaval, P., Romero, D. A., Horvath, P., and Moineau, S. (2008). Phage response to CRISPR-encoded resistance in Streptococcus thermophilus. J. Bacteriol. 190, 1390˜1400.
Dominguez, A. A., Lim, W. A., and Qi, L. S. (2016). Beyond editing: repurposing CRISPR-Cas9 for precision genome regulation and interrogation. Nat. Rev. Mol. Cell Biol. 17, 5˜15.
Dong, Guo, M., Wang, S., Zhu, Y., Wang, S., Xiong, Z., Yang, J., Xu, Z., and Huang, Z. (2017). Structural basis of CRISPR-SpyCas9 inhibition by an anti-CRISPR protein. Nature 546, 436˜439.
Esvelt, K. M., Mali, P., Braff, J. L., Moosburner, M., Yaung, S. J., and Church, G. M. (2013). Orthogonal Cas9 proteins for RNA-guided gene regulation and editing. Nat. Methods 10, 1116˜1121.
Fonfara, I., Le Rhun, A., Chylinski, K., Makarova, K. S., Lecrivain, A. L., Bzdrenga, J., Koonin, E. V., and Charpentier, E. (2014). Phylogeny of Cas9 determines functional exchangeability of dual-RNA and Cas9 among orthologous type II CRISPR-Cas systems. Nucleic Acids Res. 42, 2577˜2590.
Friedland, A. E., Baral, R., Singhal, P., Loveluck, K., Shen, S., Sanchez, M., Marco, E., Gotta, G. M., Maeder, M. L., Kennedy, E. M., et al. (2015). Characterization of Staphylococcus aureus Cas9: a smaller Cas9 for all-in-one adeno-associated virus delivery and paired nickase applications. Genome Biol. 16, 257.
Friedrich, G., and Soriano, P. (1991). Promoter traps in embryonic stem cells: a genetic screen to identify and mutate developmental genes in mice. Genes Dev. 5, 1513˜1523.
Fu, Y., Sander, J. D., Reyon, D., Cascio, V. M., and Joung, J. K. (2014). Improving CRISPR-Cas nuclease specificity using truncated guide RNAs. Nat. Biotechnol. 32, 279˜284.
Gallagher, D. N., and Haber, J. E. (2018). Repair of a Site-Specific DNA Cleavage: Old-School Lessons for Cas9-Mediated Gene Editing. ACS Chem. Biol. 13, 397˜405.
Garneau, J. E., Dupuis, M. E., Villion, M., Romero, D. A., Barrangou, R., Boyaval, P., Fremaux, C., Horvath, P., Magadan, A. H., and Moineau, S. (2010). The CRISPR/Cas bacterial immune system cleaves bacteriophage and plasmid DNA. Nature 468, 67˜71.
Gasiunas, G., Barrangou, R., Horvath, P., and Siksnys, V. (2012). Cas9-crRNA ribonucleoprotein complex mediates specific DNA cleavage for adaptive immunity in bacteria. Proc. Natl. Acad. Sci. USA 109, E2579˜2586.
Gaudelli, N. M., Komor, A. C., Rees, H. A., Packer, M. S., Badran, A. H., Bryson, D. I., and Liu, D. R. (2017). Programmable base editing of A*T to G*C in genomic DNA without DNA cleavage. Nature 551, 464˜471.
Ghanta, K., Dokshin, G., Mir, A., Krishnamurthy, P., Gneid, H., Edraki, A., Watts, J., Sontheimer, E., and Mello, C. (2018). 5′ Modifications Improve Potency and Efficacy of DNA Donors for Precision Genome Editing. Biorxiv 354480.
Gorski, S. A., Vogel, J., and Doudna, J. A. (2017). RNA-based recognition and targeting: sowing the seeds of specificity. Nat. Rev. Mol. Cell Biol. 18, 215˜228.
Harrington, L. B., Doxzen, K. W., Ma, E., Liu, J. J., Knott, G. J., Edraki, A., Garcia, B., Amrani, N., Chen, J. S., Cofsky, J. C., et al. (2017a). A Broad-Spectrum Inhibitor of CRISPR-Cas9. Cell 170, 1224˜1233.
Harrington, L. B., Paez-Espino, D., Staahl, B. T., Chen, J. S., Ma, E., Kyrpides, N. C., and Doudna, J. A. (2017b). A thermostable Cas9 with increased lifetime in human plasma. Nat. Commun. 8, 1424.
Hou, Z., Zhang, Y., Propson, N. E., Howden, S. E., Chu, L. F., Sontheimer, E. J., and Thomson, J. A. (2013). Efficient genome engineering in human pluripotent stem cells using Cas9 from Neisseria meningitidis. Proc. Natl. Acad. Sci. USA 110, 15644˜15649.
Hu, J. H., Miller, S. M., Geurts, M. H., Tang, W., Chen, L., Sun, N., Zeina, C. M., Gao, X., Rees, H. A., Lin, Z., et al. (2018). Evolved Cas9 variants with broad PAM compatibility and high DNA specificity. Nature 556, 57˜63.
Hwang, W. Y., Fu, Y., Reyon, D., Maeder, M. L., Tsai, S. Q., Sander, J. D., Peterson, R. T., Yeh, J. R., and Joung, J. K. (2013). Efficient genome editing in zebrafish using a CRISPR-Cas system. Nat. Biotechnol. 31, 227˜229.
Hynes, A. P., Rousseau, G. M., Lemay, M.-L., Horvath, P., Romero, D. A., Fremaux, C., and Moineau, S. (2017). An anti-CRISPR from a virulent streptococcal phage inhibits Streptococcus pyogenes Cas9. Nat. Microbiol. 2, 1374˜1380.
Ibraheim, R., Song, C.-Q., Mir, A., Amrani, N., Xue, W., and Sontheimer, E. J. (2018). All-in-One Adeno-associated Virus Delivery and Genome Editing by Neisseria meningitidis Cas9 in vivo. BioRxiv, doi.org/10.1101/295055.
Jiang, F., and Doudna, J. A. (2017). CRISPR-Cas9 Structures and Mechanisms. Annu. Rev. Biophys. 46, 505˜529.
Jiang, W., Bikard, D., Cox, D., Zhang, F., and Marraffini, L. A. (2013). RNA-guided editing of bacterial genomes using CRISPR-Cas systems. Nat. Biotechnol. 31, 233˜239.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816˜821.
Jinek, M., East, A., Cheng, A., Lin, S., Ma, E., and Doudna, J. (2013). RNA-programmed genome editing in human cells. eLife 2, e00471.
Karvelis, T., Gasiunas, G., Young, J., Bigelyte, G., Silanskas, A., Cigan, M., and Siksnys, V. (2015). Rapid characterization of CRISPR-Cas9 protospacer adjacent motif sequence elements. Genome Biol. 16, 253.
Keeler, A. M., ElMallah, M. K., and Flotte, T. R. (2017). Gene Therapy 2017: Progress and Future Directions. Clin. Transl. Sci. 10, 242˜248.
Kim, E., Koo, T., Park, S. W., Kim, D., Kim, K.-E., Kim, K., Cho, H.-Y., Song, D. W., Lee, K. J., Jung, M. H., et al. (2017). In vivo genome editing with a small Cas9 ortholog derived from Campylobacter jejuni. Nat. Commun. 8, 14500.
Kim, S., Kim, D., Cho, S. W., Kim, J., and Kim, J. S. (2014). Highly efficient RNA-guided genome editing in human cells via delivery of purified Cas9 ribonucleoproteins. Genome Res. 24, 1012˜1019.
Kim, B., Komor, A., Levy, J., Packer, M., Zhao, K., and Liu, D. (2017). Increasing the genome-targeting scope and precision of base editing with engineered Cas9-cytidine deaminase fusions. Nature Biotechnology 35.
Kleinstiver, B. P., Prew, M. S., Tsai, S. Q., Nguyen, N. T., Topkar, V. V., Zheng, Z., and Joung, J. K. (2015). Broadening the targeting range of Staphylococcus aureus CRISPR-Cas9 by modifying PAM recognition. Nat. Biotechnol. 33, 1293˜1298.
Kluesner, M., Nedveck, D., Lahr, W., Garbe, J., Abrahante, J., Webber, B., and Moriarity, B. (2018). EditR: A Method to Quantify Base Editing from Sanger Sequencing. The CRISPR Journal 1, 239˜250.
Koblan, L., Doman, J., Wilson, C., Levy, J., Tay, T., Newby, G., Maianti, J., Raguram, A., and Liu, D. (2018). Improving cytidine and adenine base editors by expression optimization and ancestral reconstruction. Nat Biotechnol 36, 843.
Komor, A. C., Badran, A. H., and Liu, D. R. (2017). CRISPR-Based Technologies for the Manipulation of Eukaryotic Genomes. Cell 168, 20˜36.
Komor, A. C., Kim, Y. B., Packer, M. S., Zuris, J. A., and Liu, D. R. (2016). Programmable editing of a target base in genomic DNA without double-stranded DNA cleavage. Nature 533, 420˜424.
Lee, C. M., Cradick, T. J., and Bao, G. (2016). The Neisseria meningitidis CRISPR-Cas9 system enables specific genome editing in mammalian cells. Mol. Ther. 24, 645˜654.
Lee, J., Mir, A., Edraki, A., Garcia, B., Amrani, N., Lou, H. E., Gainetdinov, I., Pawluk, A., Ibraheim, R., Gao, X. D., et al. (2018). Potent Cas9 inhibition in bacterial and human cells by new anti-CRISPR protein families. BioRxiv, biorxiv.org/content/early/2018/2006/2020/350504.
Ma, E., Harrington, L. B., O'Connell, M. R., Zhou, K., and Doudna, J. A. (2015). Single-Stranded DNA Cleavage by Divergent CRISPR-Cas9 Enzymes. Mol. Cell 60, 398˜407.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nat. Biotechnol. 31, 833˜838.
Mali, P., Yang, L., Esvelt, K. M., Aach, J., Guell, M., DiCarlo, J. E., Norville, J. E., and Church, G. M. (2013b). RNA-guided human genome engineering via Cas9. Science 339, 823-826.
Marraffini, L. A., and Sontheimer, E. J. (2008). CRISPR interference limits horizontal gene transfer in staphylococci by targeting DNA. Science 322, 1843˜1845.
Mir, A., Edraki, A., Lee, J., and Sontheimer, E. J. (2018). Type II-C CRISPR-Cas9 biology, mechanism and application. ACS Chem. Biol. 13, 357˜365.
Mojica, F. J., Diez-Villasenor, C., Garcia-Martinez, J., and Almendros, C. (2009). Short motif sequences determine the targets of the prokaryotic CRISPR defence system. Microbiology 155, 733˜740.
Paez-Espino, D., Sharon, I., Morovic, W., Stahl, B., Thomas, B. C., Barrangou, R., and Banfield, J. F. (2015). CRISPR immunity drives rapid phage genome evolution in Streptococcus thermophilus. mBio 6.
Pawluk, A., Amrani, N., Zhang, Y., Garcia, B., Hidalgo-Reyes, Y., Lee, J., Edraki, A., Shah, M., Sontheimer, E. J., Maxwell, K. L., et al. (2016). Naturally occurring off-switches for CRISPR-Cas9. Cell 167, 1829˜1838 e1829.
Pawluk, A., Bondy-Denomy, J., Cheung, V. H., Maxwell, K. L., and Davidson, A. R. (2014). A new group of phage anti-CRISPR genes inhibits the type I-E CRISPR-Cas system of Pseudomonas aeruginosa. mBio 5, e00896.
Pinello, L., Canver, M. C., Hoban, M. D., Orkin, S. H., Kohn, D. B., Bauer, D. E., and Yuan, G. C. (2016). Analyzing CRISPR genome-editing experiments with CRISPResso. Nat. Biotechnol. 34, 695˜697.
Racanelli, V., and Rehermann, B. (2006). The liver as an immunological organ. Hepatology 43, S54˜62.
Ran, F. A., Cong, L., Yan, W. X., Scott, D. A., Gootenberg, J. S., Kriz, A. J., Zetsche, B., Shalem, O., Wu, X., Makarova, K. S., et al. (2015). In vivo genome editing using Staphylococcus aureus Cas9. Nature 520, 186˜191.
Ran, F. A., Hsu, P. D., Lin, C. Y., Gootenberg, J. S., Konermann, S., Trevino, A. E., Scott, D. A., Inoue, A., Matoba, S., Zhang, Y., et al. (2013). Double nicking by RNA-guided CRISPR Cas9 for enhanced genome editing specificity. Cell 154, 1380˜1389.
Rashid, S., Curtis, D. E., Garuti, R., Anderson, N. N., Bashmakov, Y., Ho, Y. K., Hammer, R. E., Moon, Y. A., and Horton, J. D. (2005). Decreased plasma cholesterol and hypersensitivity to statins in mice lacking Pcsk9. Proc. Natl. Acad. Sci. USA 102, 5374˜5379.
Rauch, B. J., Silvis, M. R., Hultquist, J. F., Waters, C. S., McGregor, M. J., Krogan, N. J., and Bondy-Denomy, J. (2017). Inhibition of CRISPR-Cas9 with Bacteriophage Proteins. Cell 168, 150˜158 e110.
Sapranauskas, R., Gasiunas, G., Fremaux, C., Barrangou, R., Horvath, P., and Siksnys, V. (2011). The Streptococcus thermophilus CRISPR/Cas system provides immunity in Escherichia coli. Nucleic Acids Res. 39, 9275˜9282.
Schumann, K., Lin, S., Boyer, E., Simeonov, D. R., Subramaniam, M., Gate, R. E., Haliburton, G. E., Ye, C. J., Bluestone, J. A., Doudna, J. A., et al. (2015). Generation of knock-in primary human T cells using Cas9 ribonucleoproteins. Proc. Natl. Acad. Sci. USA 112, 10437-10442.
Shin, J., Jiang, F., Liu, J. J., Bray, N. L., Rauch, B. J., Baik, S. H., Nogales, E., Bondy-Denomy, J., Corn, J. E., and Doudna, J. A. (2017). Disabling Cas9 by an anti-CRISPR DNA mimic. Sci. Adv. 3, e1701620.
Tsai, S. Q., and Joung, J. K. (2016). Defining and improving the genome-wide specificities of CRISPR-Cas9 nucleases. Nat. Rev. Genet. 17, 300˜312.
Tsai, S. Q., Zheng, Z., Nguyen, N. T., Liebers, M., Topkar, V. V., Thapar, V., Wyvekens, N., Khayter, C., Iafrate, A. J., Le, L. P., et al. (2014). GUIDE-seq enables genome-wide profiling of off-target cleavage by CRISPR-Cas nucleases. Nat. Biotechnol. 33, 187˜197.
Tycko, J., Myer, V. E., and Hsu, P. D. (2016). Methods for optimizing CRISPR-Cas9 genome editing specificity. Mol. Cell 63, 355˜370.
Yang, H., and Patel, D. J. (2017). Inhibition Mechanism of an Anti-CRISPR Suppressor AcrIIA4 Targeting SpyCas9. Mol Cell 67, 117˜127 e115.
Yin, H., Song, C. Q., Suresh, S., Kwan, S. Y., Wu, Q., Walsh, S., Ding, J., Bogorad, R. L., Zhu, L. J., Wolfe, S. A., et al. (2018). Partial DNA-guided Cas9 enables genome editing with reduced off-target activity. Nat. Chem. Biol. 14, 311˜316.
Yokoyama, T., Silversides, D. W., Waymire, K. G., Kwon, B. S., Takeuchi, T., and Overbeek, P. A. (1990). Conserved cysteine to serine mutation in tyrosinase is responsible for the classical albino mutation in laboratory mice. Nucleic Acids Res. 18, 7293˜7298.
Yoon, Y., Wang, D., Tai, P. W. L., Riley, J., Gao, G., and Rivera-Perez, J. A. (2018). Streamlined ex vivo and in vivo genome editing in mouse embryos using recombinant adeno-associated viruses. Nat. Commun. 9, 412.
Zhang, Y., Heidrich, N., Ampattu, B. J., Gunderson, C. W., Seifert, H. S., Schoen, C., Vogel, J., and Sontheimer, E. J. (2013). Processing-independent CRISPR RNAs limit natural transformation in Neisseria meningitidis. Mol. Cell 50, 488˜503.
Zhang, Y., Rajan, R., Seifert, H. S., Mondragón, A., and Sontheimer, E. J. (2015). DNase H activity of Neisseria meningitidis Cas9. Mol. Cell 60, 242˜255.
Zhang, Z., Theurkauf, W. E., Weng, Z., and Zamore, P. D. (2012). Strand-specific libraries for high throughput RNA sequencing (RNA-Seq) prepared without poly(A) selection. Silence 3, 9.
Zhu, L. J., Holmes, B. R., Aronin, N., and Brodsky, M. H. (2014). CRISPRseek: a bioconductor package to identify target-specific guide RNAs for CRISPR-Cas9 genome-editing systems. PLoS One 9, e108424.
Zhu, L. J., Lawrence, M., Gupta, A., Pag6s, H., Kucukural, A., Garber, M., and Wolfe, S. A. (2017). GUIDEseq: a bioconductor package to analyze GUIDE-Seq datasets for CRISPR-Cas nucleases. BMC Genomics 18, 379.
Zuris, J. A., Thompson, D. B., Shu, Y., Guilinger, J. P., Bessen, J. L., Hu, J. H., Maeder, M. L., Joung, J. K., Chen, Z.-Y., and Liu, D. R. (2015). Cationic lipid-mediated delivery of proteins enables efficient protein-based genome editing in vitro and in vivo. Nat. Biotechnol. 33, 73˜80.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biological control, biochemistry, molecular biology, entomology, plankton, fishery systems, and fresh water ecology, or related fields are intended to be within the scope of the following claims.

1. A Neisseria meningitidis (Nme) 2 Cas9 (Nme2Cas9) variant comprising an amino acid substitution at one or more positions selected from the group consisting of E520, D873, D418, E471, D442, E844, E443, D470, E585, E552, D451, E587, E508, E932, D56, D1048, E1079, D660, E887, T72, and E186.

2. (canceled)

3. The Nme2Cas9 variant of claim 1, comprising:
amino acid substitutions at positions E932 and D873; E932 and D56; E932 and E520; E932 and D1048; D873 and D56; D873 and E520; D873 and D1048; D56 and E520; D56 and D1048; E520 and D1048; E932, D873, and D56; E932, D873, and E520; E932, D873, and D1048; E932, D56, and E520; E932, D56, and D1048; E932, E520, and D1048; D873, D56, and E520; D873, D56, and D1048; D873, E520, and D1048; D56, E520, and D1048; E932, D873, D56, and E520; E932, D873, D56, and D1048; E932, D56, E520, and D1048; D873, D56, E520, and D1048; or E932, D873, D56, E520, and D1048; or
amino acid substitutions E932R and D873R; E932R and D56R: E932R and E520R: E932R and D1048R: D873R and D56R: D873R and E520R: D873R and D1048R: D56R and E520R: D56R and D1048R: E520R and D1048R: E932R, D873R, and D56R: E932R, D873R, and E520R: E932R, D873R, and D1048R: E932R, D56R, and E520R: E932R, D56R, and D1048R: E932R, E520R, and D1048R: D873R, D56R, and E520R: D873R, D56R, and D1048R: D873R, E520R, and D1048R: D56R, E520R, and D1048R: E932R, D873R, D56R, and E520R: E932R, D873R, D56R, and D1048R: E932R, D56R, E520R, and D1048R: D873R, D56R, E520R, and D1048R: or E932R, D873R, D56R, E520R, and D1048R.

4-9. (canceled)

10. The Nme2Cas9 variant of any-ene-ef cais˜1˜9claim 1, wherein the Nme2Cas9 variant comprises a protospacer adjacent motif interacting domain (PID) that interacts with an N₄CC nucleotide sequence, an N₄CA nucleotide sequence, an N₄CG nucleotide sequence, an N₄CT nucleotide sequence, or an N₄C nucleotide sequence, optionally wherein the PID is an Nme2Cas9 PID or an SmuCas9 PID, optionally wherein:
the Nme2Cas9 PID comprises an amino acid sequence set forth in SEQ ID NO:27 (DNGDMVRVDVFCKVDKKGKNQYFIVPIYAWQVAENILPDIDCKGYRIDDSYTFCFSLH KYDLIAFQKDEKSKVEFAYYINCDSSNGRFYLAWHDKGSKEQQFRISTQNLVLIQKYQV NELGKEIRPCRLKKRPPVR); or
the SmuCas9 PID comprises an amino acid sequence set forth in SEQ ID NO:28 (DNATMVRVDVYTKAGKNYLVPVYVWQVAQGILPNRAVTSGKSEADWDLIDESFEFKF SLSRGDLVEMISNKGRIFGYYNGLDRANGSIGIREHDLEKSKGKDGVHRVGVKTATAFN KYHVDPLGKEIHRCSSEPRPTLKIKSKK).

11-13. (canceled)

14. The Nme2Cas9 variant of claim 1, wherein the one or more positions are relative to an amino acid sequence of SEQ ID NO: 1 or SEQ ID NO: 2.

15. The Nme2Cas9 variant of claim 1, further comprising a nucleotide base editor (NBE) domain fused to the Nme2Cas9 variant.

16. The Nme2Cas9 variant of claim 15, wherein the NBE domain is an inlaid NBE domain inserted into the Nme2Cas9 variant.

17-22. (canceled)

23. The Nme2Cas9 variant of claim 16, wherein the inlaid NBE domain is flanked at an inlaid NBE domain N-terminus and/or an inlaid NBE domain C-terminus by an amino acid linker, optionally wherein:
the amino acid linker comprises a (GGS)ₙ (SEQ ID NO:40) linker, wherein n corresponds to 1˜6;
the amino acid linker comprises GGSGGSGGSGGSGGSGGSGG (SEQ ID NO: 15);
the amino acid linker comprises GSSGSETPGTSESATPESSG (SEQ ID NO: 21); or
the inlaid NBE domain is flanked at the inlaid NBE domain N-terminus by GGSGGSGGSGGSGGSGGSGG (SEQ ID NO: 15) and at the inlaid NBE domain C-terminus by GSSGSETPGTSESATPESSG (SEQ ID NO: 21).

24-28. (canceled)

29. The Nme2Cas9 variant of claim 16, wherein the inlaid NBE domain is linked via an amino acid linker to an N-terminus of the Nme2Cas9 variant or an C-terminus of the Nme2Cas9 variant, optionally wherein:
the amino acid linker comprises a (GGS)ₙ (SEQ ID NO:40) linker, wherein n corresponds to 1˜6;
the amino acid linker comprises GGSGGSGGSGGSGGSGGSGG (SEQ ID NO: 15);
the amino acid linker comprises GSSGSETPGTSESATPESSG (SEQ ID NO: 21); or
the amino acid linker comprises ED.

30-34. (canceled)

35. The Nme2Cas9 variant of claim 16, wherein the inlaid NBE domain is an adenine base editor (ABE) domain, optionally wherein the ABE domain is an inlaid adenosine deaminase protein domain, optionally wherein the inlaid adenosine deaminase protein domain is an adenosine deaminase 8e protein domain (TadA8e), optionally wherein the TadA8e comprises an amino acid sequence set forth in SEQ ID NO: 9 (SEVEFSHEYWMRHALTLAKRARDEREVPVGAVLVLNNRVIGEGWNRAIGLHDPTAHA EIMALRQGGLVMQNYRLIDATLYVTFEPCVMCAGAMIHSRIGRVVFGVRNSKRGAAGS LMNVLNYPGMNHRVEITEGILADECAALLCDFYRMPRQVFNAQKKAQSSIN).

36-38. (canceled)

39. The Nme2Cas9 variant of claim 16, wherein the inlaid NBE domain is a cytidine base editor (CBE) domain, optionally wherein the inlaid CBE domain is an inlaid cytosine deaminase protein domain, optionally wherein the cytosine deaminase protein domain is evoFERNY or rAPOBEC1, optionally wherein:
the evoFERNY comprises an amino acid sequence set forth in SEQ ID NO: 13 (FERNYDPRELRKETYLLYEIKWGKSGKLWRHWCQNNRTQHAEVYFLENIFNARRFNPS THCSITWYLSWSPCAECSQKIVDFLKEHPNVNLEIYVARLYYPENERNRQGLRDLVNSG VTIRIMDLPDYNYCWKTFVSDQGGDEDYWPGHFAPWIKQYSLKL), or
the rAPOBEC1 comprises an amino acid sequence set forth in SEQ ID NO: 11 (SSETGPVAVDPTLRRRIEPHEFEVFFDPRELRKETCLLYEINWGGRHSIWRHTSQNTNKH VEVNFIEKFTTERYFCPNTRCSITWFLSWSPCGECSRAITEFLSRYPHVTLFIYIARLYHHA DPRNRQGLRDLISSGVTIQIMTEQESGYCWRNFVNYSPSNEAHWPRYPHLWVRLYVLEL YCIILGLPPCLNILRRKQPQLTFFTIALQSCHYQRLPPHILWATGLK).

40-43. (canceled)

44. The Nme2Cas9 variant of claim 1, further comprising one or more nuclear localization signals (NLS), optionally wherein:
the one or more NLS are any one or more of a nucleoplasmin NLS, an SV40 NLS, or a C-myc NL;
the one or more NLS comprise an amino acid sequence selected from the group consisting of MKRTADGSEFESPKKKRKV (SEQ ID NO:30), KRTADGSEFEPKKKRKV (SEQ ID NO:31), MKRPAATKKAGQAKKKK (SEQ ID NO:32), KRPAATKKAGQAKKKK (SEQ ID NO:33), MPKKKRKV (SEQ ID NO:34), and PKKKRKV (SEQ ID NO:35); or
the one or more NLS are positioned at an N-terminus and/or a C-terminus of the Nme2Cas9 variant.

45-49. (canceled)

50. A polynucleotide encoding the Nme2Cas9 variant of claim 1.

51. (canceled)

52. A vector comprising the polynucleotide of claim 50.

53. A viral vector comprising the polynucleotide of claim 50.

54. (canceled)

55. An adeno-associated virus (AAV) comprising the polynucleotide of claim 50.

56. A genome editing system comprising the Nme2Cas9 variant of claim 1 and a guide RNA (gRNA).

57. The genome editing system of claim 56, wherein the gRNA comprises:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence.

58. The genome editing system of claim 56, wherein the gRNA comprises at least one modified nucleotide, optionally wherein the at least one modified nucleotide comprises a modification of a ribose group, a phosphate group, a nucleobase, or a combination thereof, optionally wherein:
the modification of the ribose group is independently selected from the group consisting of a 2′-O-methyl, a 2′-fluoro, a 2′-deoxy, a 2′-O-(2-methoxyethyl) (MOE), a 2′-NH2 (2′-amino), a 4′-thio, a bicyclic nucleotide, a locked nucleic acid (LNA), a 2′-(S)-constrained ethyl (S-cEt), a constrained MOE, and a 2′˜0,4′-C-aminomethylene bridged nucleic acid (2′,4′-BNA^{NC});
the modification of the phosphate group is independently selected from the group consisting of a phosphorothioate, a phosphonoacetate (PACE), a thiophosphonoacetate (thioPACE), an amide, a triazole, a phosphonate, and a phosphotriester modification: or
the modification of the nucleobase group is independently selected from the group consisting of a 2-thiouridine, a 4-thiouridine, a N⁶-methyladenosine, a pseudouridine, 2,6-diaminopurine, an inosine, a thymidine, a 5-methylcytosine, a 5-substituted pyrimidine, an isoguanine, an isocytosine, and halogenated aromatic groups.

59-65. (canceled)

66. A method of editing a genome, comprising:
(a) introducing into the genome the genome editing system of claim 56; and
(b) incubating the genome editing system with the genome for a time sufficient to edit the genome.

67-68. (canceled)

69. A fusion protein comprising a Neisseria meningitidis (Nme) 2 Cas9 (Nme2Cas9) protein and an inlaid nucleotide base editor (NBE) domain,
wherein the inlaid NBE domain is flanked at an inlaid NBE domain N-terminus and/or an inlaid NBE domain C-terminus by an amino acid linker, or a linker is absent, and
wherein the total number of amino acid linker residues is less than 40 amino acids.

70-79. (canceled)

80. The fusion protein of claim 69, wherein:
A:
the amino acid linker comprises a sequence selected from the group consisting of: GGSGGSGGSGGSGGSGGSGG (SEQ ID NO: 15), SGGSGGSGGS (SEQ ID NO: 17), GGSGG (SEQ ID NO: 19), GSSGSETPGTSESATPESSG (SEQ ID NO: 21), ETPGTSESAT (SEQ ID NO: 23), and GTSES (SEQ ID NO: 25;
B:
the amino acid linker is present at the N-terminus of the inlaid NBE domain and comprises GGSGGSGGSGGSGGSGGSGG (SEQ ID NO: 15), and optionally the amino acid linker is present at the C-terminus of the inlaid NBE domain and comprises ETPGTSESAT (SEQ ID NO: 23) or GTSES(SEQ ID NO: 25):
C:
the amino acid linker is present at the N-terminus of the inlaid NBE domain and comprises SGGSGGSGGS (SEQ ID NO: 17), and optionally the amino acid linker is present at the C-terminus of the inlaid NBE domain and comprises GSSGSETPGTSESATPESSG (SEQ ID NO: 21), ETPGTSESAT (SEQ ID NO: 23) or GTSES (SEQ ID NO: 25);
D:
the amino acid linker is present at the N-terminus of the inlaid NBE domain and comprises GGSGG (SEQ ID NO: 19), and optionally the amino acid linker is present at the C-terminus of the inlaid NBE domain and comprises GSSGSETPGTSESATPESSG (SEQ ID NO: 21), ETPGTSESAT (SEQ ID NO: 23), or GTSES (SEQ ID NO: 25):
E:
the amino acid linker is absent at the N-terminus of the inlaid NBE domain, and optionally the amino acid linker is present at the C-terminus of the inlaid NBE domain and comprises GSSGSETPGTSESATPESSG (SEQ ID NO: 21), ETPGTSESAT (SEQ ID NO: 23), or GTSES (SEQ ID NO: 25);
F:
the amino acid linker is present at the C-terminus of the inlaid NBE domain and comprises GSSGSETPGTSESATPESSG (SEQ ID NO: 21), and optionally the amino acid linker is present at the N-terminus of the inlaid NBE domain and comprises SGGSGGSGGS (SEQ ID NO: 17) or GGSGG (SEQ ID NO: 19);
G:
the amino acid linker is present at the C-terminus of the inlaid NBE domain and comprises ETPGTSESAT (SEQ ID NO: 23), and optionally the amino acid linker is present at the N-terminus of the inlaid NBE domain and comprises GGSGGSGGSGGSGGSGGSGG (SEQ ID NO: 15), SGGSGGSGGS (SEQ ID NO: 17), or GGSGG (SEQ ID NO: 19):
H:
the amino acid linker is present at the C-terminus of the inlaid NBE domain and comprises GTSES (SEQ ID NO: 25), and optionally the amino acid linker is present at the N-terminus of the inlaid NBE domain and comprises GGSGGSGGSGGSGGSGGSGG (SEQ ID NO: 15), SGGSGGSGGS (SEQ ID NO: 17), or GGSGG (SEQ ID NO: 19); or
I:
the amino acid linker is absent at the C-terminus of the inlaid NBE domain, and optionally the amino acid linker is present at the N-terminus of the inlaid NBE domain and comprises GGSGGSGGSGGSGGSGGSGG (SEQ ID NO: 15), SGGSGGSGGS (SEQ ID NO: 17), or GGSGG (SEQ ID NO: 19).

81-89. (canceled)

90. A polynucleotide encoding the fusion protein of claim 69.

91. (canceled)

92. A vector comprising the polynucleotide of claim 90.

93. A viral vector comprising the polynucleotide of claim 90.

94. (canceled)

95. An adeno-associated virus (AAV) comprising the polynucleotide of claim 90.


SEQUENCE LISTING (115 sequences)

SEQ ID NO: 1 - protein, 1082 aa, synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 2 - protein, 1090 aa, synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 3 - DNA, 3270 bp, synthetic construct
  source 1..3270: mol_type = other DNA
atggccgccttcaagcctaacccaatcaattacatcctgggactggacatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaag

SEQ ID NO: 4 - protein, 1130 aa, synthetic construct
MKRTADGSEFESPKKKRKVEDMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 5 - DNA, 3390 bp, synthetic construct
  source 1..3390: mol_type = other DNA
atgaaacggacagccgacggaagcgagttcgagtcaccaaagaagaagcggaaagtcgaagatatggccgccttcaagcctaacccaatcaattacatcctgggactggacatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtc

SEQ ID NO: 6 - protein, 1258 aa, synthetic construct
  VARIANT 460: note = X can be any amino acid
  VARIANT 460: note = X can be repeated one to twenty times or can be absent
  VARIANT 292: note = X can be any amino acid
  VARIANT 292: note = X can be repeated one to twenty times or can be absent
MAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQXSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINXGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 7 - DNA, 3771 bp, synthetic construct
  source 1..3771: mol_type = other DNA
atggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagntctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacncggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaag

SEQ ID NO: 8 - protein, 1258 aa, synthetic construct
  VARIANT 796: note = X can be any amino acid
  VARIANT 796: note = X can be repeated one to twenty times or can be absent
  VARIANT 964: note = X can be any amino acid
  VARIANT 964: note = X can be repeated one to twenty times or can be absent
MAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPXSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINXEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 9 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 10 - DNA, 498 bp, synthetic construct
  source 1..498: mol_type = other DNA
tctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaac

SEQ ID NO: 11 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 12 - DNA, 684 bp, synthetic construct
  source 1..684: mol_type = other DNA
agctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 13 - protein, 161 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 14 - DNA, 483 bp, synthetic construct
  source 1..483: mol_type = other DNA
tttgagaggaactacgacccccgggagctgagaaaggagacatacctgctgtatgagatcaagtggggcaagtccggcaagctgtggaggcactggtgccagaacaatcgcacacagcacgccgaggtgtacttcctggagaacatctttaatgcccggagattcaatccatctacccactgtagcatcacatggtatctgagctggtccccctgcgccgagtgttctcagaagatcgtggatttcctgaaggagcaccctaacgtgaatctggagatctatgtggcccggctgtactatccagagaacgagaggaataggcagggcctgcgggatctggtgaattccggcgtgaccatcagaatcatggacctgccagattacaactattgctggaagaccttcgtgagcgatcagggaggcgacgaggattactggccaggacacttcgccccttggatcaagcagtatagcctgaagctg

SEQ ID NO: 15 - protein, 20 aa, synthetic construct
GGSGGSGGSGGSGGSGGSGG

SEQ ID NO: 16 - DNA, 60 bp, synthetic construct
  source 1..60: mol_type = other DNA
ggcggatcaggaggctctggcggttcaggtggatcaggcggtagcggaggttcaggtggt

SEQ ID NO: 17 - protein, 10 aa, synthetic construct
SGGSGGSGGS

SEQ ID NO: 18 - DNA, 30 bp, synthetic construct
  source 1..30: mol_type = other DNA
tctggcggttcaggtggatcaggcggtagc

SEQ ID NO: 19 - protein, 5 aa, synthetic construct
GGSGG

SEQ ID NO: 20 - DNA, 15 bp, synthetic construct
  source 1..15: mol_type = other DNA
ggcggttcaggtgga

SEQ ID NO: 21 - protein, 20 aa, synthetic construct
GSSGSETPGTSESATPESSG

SEQ ID NO: 22 - DNA, 60 bp, synthetic construct
  source 1..60: mol_type = other DNA
ggctcctctggctctgagacacctggcacaagcgagagcgcaacacctgaaagcagcggc

SEQ ID NO: 23 - protein, 10 aa, synthetic construct
ETPGTSESAT

SEQ ID NO: 24 - DNA, 30 bp, synthetic construct
  source 1..30: mol_type = other DNA
gagacacctggcacaagcgagagcgcaaca

SEQ ID NO: 25 - protein, 5 aa, synthetic construct
GTSES

SEQ ID NO: 26 - DNA, 15 bp, synthetic construct
  source 1..15: mol_type = other DNA
ggcacaagcgagagc

SEQ ID NO: 27 - protein, 136 aa, synthetic construct
DNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 28 - protein, 144 aa, synthetic construct
DNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 29 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 30 - protein, 19 aa, synthetic construct
MKRTADGSEFESPKKKRKV

SEQ ID NO: 31 - protein, 17 aa, synthetic construct
KRTADGSEFEPKKKRKV

SEQ ID NO: 32 - protein, 17 aa, synthetic construct
MKRPAATKKAGQAKKKK

SEQ ID NO: 33 - protein, 16 aa, synthetic construct
KRPAATKKAGQAKKKK

SEQ ID NO: 34 - protein, 8 aa, synthetic construct
MPKKKRKV

SEQ ID NO: 35 - protein, 7 aa, synthetic construct
PKKKRKV

SEQ ID NO: 36 - protein, 6 aa, synthetic construct
GGSGGS

SEQ ID NO: 37 - protein, 9 aa, synthetic construct
GGSGGSGGS

SEQ ID NO: 38 - protein, 12 aa, synthetic construct
GGSGGSGGSGGS

SEQ ID NO: 39 - protein, 15 aa, synthetic construct
GGSGGSGGSGGSGGS

SEQ ID NO: 40 - protein, 6 aa, synthetic construct
  VARIANT 1..3: note = The amino acid residues can be repeated one or four times
GGSGGS

SEQ ID NO: 41 - protein, 6 aa, synthetic construct
  VARIANT 1..3: note = The amino acid residues can be repeated one or five times
GGSGGS

SEQ ID NO: 42 - protein, 6 aa, synthetic construct
HHHHHH

SEQ ID NO: 43 - DNA, 52 bp, synthetic construct
  source 1..52: mol_type = other DNA
cagcttggcggtctaggtgccctcgtagggtcggccctcaccctcaccctcg

SEQ ID NO: 44 - DNA, 52 bp, synthetic construct
  source 1..52: mol_type = other DNA
gtcgaaccgccagatccacgggagcatcccagccgggagtgggagtgggagc

SEQ ID NO: 45 - DNA, 40 bp, synthetic construct
  source 1..40: mol_type = other DNA
gacacgctgaacttgtggccgtttacgtcgccgtccagct

SEQ ID NO: 46 - DNA, 40 bp, synthetic construct
  source 1..40: mol_type = other DNA
ctgtgcgacttgaacaccggcaaatgcagcggcaggtcga

SEQ ID NO: 47 - DNA, 23 bp, synthetic construct
  source 1..23: mol_type = other DNA
cagaccagtaaggaagcaggtga

SEQ ID NO: 48 - DNA, 23 bp, synthetic construct
  source 1..23: mol_type = other DNA
agacttatccacaagagtcagat

SEQ ID NO: 49 - DNA, 23 bp, synthetic construct
  source 1..23: mol_type = other DNA
tgttagaagaaccagatagacag

SEQ ID NO: 50 - DNA, 23 bp, synthetic construct
  source 1..23: mol_type = other DNA
gcaagaacacaagaggaagagag

SEQ ID NO: 51 - DNA, 23 bp, synthetic construct
  source 1..23: mol_type = other DNA
attccaaccaaacacccgccccc

SEQ ID NO: 52 - DNA, 23 bp, synthetic construct
  source 1..23: mol_type = other DNA
gagtcgacagaggacggtggtgg

SEQ ID NO: 53 - DNA, 23 bp, synthetic construct
  source 1..23: mol_type = other DNA
gctgcaaaacaagaggaggcaag

SEQ ID NO: 54 - DNA, 23 bp, synthetic construct
  source 1..23: mol_type = other DNA
acccagaggtagaaatggagagg

SEQ ID NO: 55 - DNA, 23 bp, synthetic construct
  source 1..23: mol_type = other DNA
ttctccagtcacaagctgtgctc

SEQ ID NO: 56 - DNA, 23 bp, synthetic construct
  source 1..23: mol_type = other DNA
ggggtaaaaaaggagagggtccc

SEQ ID NO: 57 - DNA, 23 bp, synthetic construct
  source 1..23: mol_type = other DNA
tggtggaagaaggagcagtatga

SEQ ID NO: 58 - DNA, 23 bp, synthetic construct
  source 1..23: mol_type = other DNA
tgaccaacgaaaaaaaagggact

SEQ ID NO: 59 - DNA, 24 bp, synthetic construct
  source 1..24: mol_type = other DNA
ccagaccagtaaggaagcaggtga

SEQ ID NO: 60 - DNA, 24 bp, synthetic construct
  source 1..24: mol_type = other DNA
cagacttatccacaagagtcagat

SEQ ID NO: 61 - DNA, 24 bp, synthetic construct
  source 1..24: mol_type = other DNA
ctgttagaagaaccagatagacag

SEQ ID NO: 62 - DNA, 24 bp, synthetic construct
  source 1..24: mol_type = other DNA
agcaagaacacaagaggaagagag

SEQ ID NO: 63 - DNA, 24 bp, synthetic construct
  source 1..24: mol_type = other DNA
gattccaaccaaacacccgccccc

SEQ ID NO: 64 - DNA, 24 bp, synthetic construct
  source 1..24: mol_type = other DNA
ggagtcgacagaggacggtggtgg

SEQ ID NO: 65 - DNA, 24 bp, synthetic construct
  source 1..24: mol_type = other DNA
ggctgcaaaacaagaggaggcaag

SEQ ID NO: 66 - DNA, 24 bp, synthetic construct
  source 1..24: mol_type = other DNA
gacccagaggtagaaatggagagg

SEQ ID NO: 67 - DNA, 24 bp, synthetic construct
  source 1..24: mol_type = other DNA
attctccagtcacaagctgtgctc

SEQ ID NO: 68 - DNA, 24 bp, synthetic construct
  source 1..24: mol_type = other DNA
aggggtaaaaaaggagagggtccc

SEQ ID NO: 69 - DNA, 24 bp, synthetic construct
  source 1..24: mol_type = other DNA
atggtggaagaaggagcagtatga

SEQ ID NO: 70 - DNA, 24 bp, synthetic construct
  source 1..24: mol_type = other DNA
ctgaccaacgaaaaaaaagggact

SEQ ID NO: 71 - DNA, 3987 bp, synthetic construct
  source 1..3987: mol_type = other DNA
aaacggacagccgacggaagcgagttcgagtcaccaaagaagaagcggaaagtcggcggtagcggcggaggcagcggtggcggcagcggctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcaatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 72 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 73 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 74 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcgataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 75 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagaggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggctttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 76 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcgatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 77 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggccgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 78 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 79 - DNA, 3981 bp, synthetic construct
  source 1..3981: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcgggaggataaaagaaccgccgacggcagcgaattcgagcccaagaagaagaggaaagtc

SEQ ID NO: 80 - DNA, 4011 bp, synthetic construct
  source 1..4011: mol_type = other DNA
aaacggacagccgacggaagcgagttcgagtcaccaaagaagaagcggaaagtcggcggtagcggcggaggcagcggtggcggcagcggctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcaatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtc

SEQ ID NO: 81 - DNA, 4005 bp, synthetic construct
  source 1..4005: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtc

SEQ ID NO: 82 - DNA, 4005 bp, synthetic construct
  source 1..4005: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtc

SEQ ID NO: 83 - DNA, 4005 bp, synthetic construct
  source 1..4005: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtc

SEQ ID NO: 84 - DNA, 3954 bp, synthetic construct
  source 1..3954: mol_type = other DNA
aaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactctggaggatctagcggaggatcctctggcagcgagacaccaggaacaagcgagtcagcaacaccagagagcagtggcggcagcagcggcggcagcgcagcattcaagtcaaacccaatcaattacatcctgggactggcaatcggaatcgcatccgtgggatgggctatggtggagatcgacgaggaggggaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcacgagcttgccaaacaccccctggcagctgagagcagccgccctggacaggaagctgacaccactggagtggtctgccgtgctgctgcacctgatcaagcaccgcggctacctgagccagcggaagaacgagggagagacagcagccaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctggggcactgcaccctcgagcctacagagccaaaggccgccaagaacacctacacagccgagcggtttatctggctgacaaagctgaacaatctgagaatcctggagcagggatccgagaggccactgaccgacacagagaggtccaccctgatggatgagccttaccggaagtctaaactgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgttcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgcgaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaagagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccagtagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtggtcgcctctaatggccagatcacaaacctgctgagggggttttggagactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccgtcgataaggagacaggcaaggtgctgtaccagaagacccacttcccccagccttgggagttctttgcccaggaagttatgatccgggtgttcggcaagccagacggcaagcctgagtttgaggaggccgataccccagagaagctgaggacactgctggcagagaagctgtctagcaggccagaggcagtgcacgagtacgtgaccccgctgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccagaagagcggcgtgctgctgaataagaagaacgcctacacaatcgccgacaatggtgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctagcggcgggttctacctggcctggcacgataagggcagcagggagcagcggtttcgcatctccacccagaatctggcgctgatccagaagtatcaggtgaacgagctgggcaaggagatcaggccatgtcggctgaagaagcgcccacccgtgcggtctggcggctcaaaaagaaccgccgacggcagcgaattcgagcccaagaagaagaggaaagtc

SEQ ID NO: 85 - DNA, 4221 bp, synthetic construct
  source 1..4221: mol_type = other DNA
aaacggacagccgacggaagcgagttcgagtcaccaaagaagaagcggaaagtcagttttgagaggaactacgacccccgggagctgagaaaggagacatacctgctgtatgagatcaagtggggcaagtccggcaagctgtggaggcactggtgccagaacaatcgcacacagcacgccgaggtgtacttcctggagaacatctttaatgcccggagattcaatccatctacccactgtagcatcacatggtatctgagctggtccccctgcgccgagtgttctcagaagatcgtggatttcctgaaggagcaccctaacgtgaatctggagatctatgtggcccggctgtactatccagagaacgagaggaataggcagggcctgcgggatctggtgaattccggcgtgaccatcagaatcatggacctgccagattacaactattgctggaagaccttcgtgagcgatcagggaggcgacgaggattactggccaggacacttcgccccttggatcaagcagtatagcctgaagctgtctggcggatctagcggaggatcctctggcagcgagacaccaggaacaagcgagtcagcaacaccagagagcagtggcggcagcagcggcggcagcgccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 86 - DNA, 4251 bp, synthetic construct
  source 1..4251: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggcggatcaggaggctctggcggttcaggtggatcaggcggtagcggaggttcaggtggttttgagaggaactacgacccccgggagctgagaaaggagacatacctgctgtatgagatcaagtggggcaagtccggcaagctgtggaggcactggtgccagaacaatcgcacacagcacgccgaggtgtacttcctggagaacatctttaatgcccggagattcaatccatctacccactgtagcatcacatggtatctgagctggtccccctgcgccgagtgttctcagaagatcgtggatttcctgaaggagcaccctaacgtgaatctggagatctatgtggcccggctgtactatccagagaacgagaggaataggcagggcctgcgggatctggtgaattccggcgtgaccatcagaatcatggacctgccagattacaactattgctggaagaccttcgtgagcgatcagggaggcgacgaggattactggccaggacacttcgccccttggatcaagcagtatagcctgaagctgggctcctctggctctgagacacctggcacaagcgagagcgcaacacctgaaagcagcggcggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 87 - DNA, 4251 bp, synthetic construct
  source 1..4251: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagggcggatcaggaggctctggcggttcaggtggatcaggcggtagcggaggttcaggtggttttgagaggaactacgacccccgggagctgagaaaggagacatacctgctgtatgagatcaagtggggcaagtccggcaagctgtggaggcactggtgccagaacaatcgcacacagcacgccgaggtgtacttcctggagaacatctttaatgcccggagattcaatccatctacccactgtagcatcacatggtatctgagctggtccccctgcgccgagtgttctcagaagatcgtggatttcctgaaggagcaccctaacgtgaatctggagatctatgtggcccggctgtactatccagagaacgagaggaataggcagggcctgcgggatctggtgaattccggcgtgaccatcagaatcatggacctgccagattacaactattgctggaagaccttcgtgagcgatcagggaggcgacgaggattactggccaggacacttcgccccttggatcaagcagtatagcctgaagctgggctcctctggctctgagacacctggcacaagcgagagcgcaacacctgaaagcagcggcacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 88 - DNA, 4251 bp, synthetic construct
  source 1..4251: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctggcggatcaggaggctctggcggttcaggtggatcaggcggtagcggaggttcaggtggttttgagaggaactacgacccccgggagctgagaaaggagacatacctgctgtatgagatcaagtggggcaagtccggcaagctgtggaggcactggtgccagaacaatcgcacacagcacgccgaggtgtacttcctggagaacatctttaatgcccggagattcaatccatctacccactgtagcatcacatggtatctgagctggtccccctgcgccgagtgttctcagaagatcgtggatttcctgaaggagcaccctaacgtgaatctggagatctatgtggcccggctgtactatccagagaacgagaggaataggcagggcctgcgggatctggtgaattccggcgtgaccatcagaatcatggacctgccagattacaactattgctggaagaccttcgtgagcgatcagggaggcgacgaggattactggccaggacacttcgccccttggatcaagcagtatagcctgaagctgggctcctctggctctgagacacctggcacaagcgagagcgcaacacctgaaagcagcggcgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 89 - DNA, 4422 bp, synthetic construct
  source 1..4422: mol_type = other DNA
aaacggacagccgacggaagcgagttcgagtcaccaaagaagaagcggaaagtcagtagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggcggatctagcggaggatcctctggcagcgagacaccaggaacaagcgagtcagcaacaccagagagcagtggcggcagcagcggcggcagcgccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 90 - DNA, 4452 bp, synthetic construct
  source 1..4452: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggcggatcaggaggctctggcggttcaggtggatcaggcggtagcggaggttcaggtggtagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaggctcctctggctctgagacacctggcacaagcgagagcgcaacacctgaaagcagcggcggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 91 - DNA, 4452 bp, synthetic construct
  source 1..4452: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagggcggatcaggaggctctggcggttcaggtggatcaggcggtagcggaggttcaggtggtagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaggctcctctggctctgagacacctggcacaagcgagagcgcaacacctgaaagcagcggcacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 92 - DNA, 4452 bp, synthetic construct
  source 1..4452: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctggcggatcaggaggctctggcggttcaggtggatcaggcggtagcggaggttcaggtggtagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaggctcctctggctctgagacacctggcacaagcgagagcgcaacacctgaaagcagcggcgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 93 - DNA, 30 bp, synthetic construct
  source 1..30: mol_type = other DNA
gaatggcaggcggaggttgtactgggggcc

SEQ ID NO: 94 - DNA, 30 bp, synthetic construct
  source 1..30: mol_type = other DNA
aaacggaagccgcacgtctcactagtaccc

SEQ ID NO: 95 - DNA, 30 bp, synthetic construct
  source 1..30: mol_type = other DNA
tgaggaccgccctgggcctgggagaatccc

SEQ ID NO: 96 - DNA, 30 bp, synthetic construct
  source 1..30: mol_type = other DNA
gaagaaacgccatgggcctgggagactccc

SEQ ID NO: 97 - DNA, 30 bp, synthetic construct
  source 1..30: mol_type = other DNA
tgcagatcccacaggcgccctggccagtcg

SEQ ID NO: 98 - DNA, 30 bp, synthetic construct
  source 1..30: mol_type = other DNA
tgcatttcccacaggcgccctggcgatgcc

SEQ ID NO: 99 - DNA, 30 bp, synthetic construct
  source 1..30: mol_type = other DNA
agtctccgctttaacccccacctccagccg

SEQ ID NO: 100 - DNA, 30 bp, synthetic construct
  source 1..30: mol_type = other DNA
ttctgctttaacccccacctcctatcatac

SEQ ID NO: 101 - DNA, 30 bp, synthetic construct
  source 1..30: mol_type = other DNA
gaaggaccaccctaggcctgggagactccc

SEQ ID NO: 102 - protein, 16 aa, synthetic construct
EGEGEGRPYEGTTAKL

SEQ ID NO: 103 - protein, 14 aa, synthetic construct
ELDGDVNGHKFSVS

SEQ ID NO: 104 - RNA, 24 nt, synthetic construct
  source 1..24: mol_type = other RNA
gcggtctaggtgccctcgtagggt

SEQ ID NO: 105 - RNA, 24 nt, synthetic construct
  source 1..24: mol_type = other RNA
ggcgtctaggtgccctcgtagggt

SEQ ID NO: 106 - RNA, 24 nt, synthetic construct
  source 1..24: mol_type = other RNA
gcgcactaggtgccctcgtagggt

SEQ ID NO: 107 - RNA, 24 nt, synthetic construct
  source 1..24: mol_type = other RNA
gcggtgaaggtgccctcgtagggt

SEQ ID NO: 108 - RNA, 24 nt, synthetic construct
  source 1..24: mol_type = other RNA
gcggtcttcgtgccctcgtagggt

SEQ ID NO: 109 - RNA, 24 nt, synthetic construct
  source 1..24: mol_type = other RNA
gcggtctagcagccctcgtagggt

SEQ ID NO: 110 - RNA, 24 nt, synthetic construct
  source 1..24: mol_type = other RNA
gcggtctaggtcgcctcgtagggt

SEQ ID NO: 111 - RNA, 24 nt, synthetic construct
  source 1..24: mol_type = other RNA
gcggtctaggtgcggtcgtagggt

SEQ ID NO: 112 - RNA, 24 nt, synthetic construct
  source 1..24: mol_type = other RNA
gcggtctaggtgcccaggtagggt

SEQ ID NO: 113 - RNA, 24 nt, synthetic construct
  source 1..24: mol_type = other RNA
gcggtctaggtgccctccaagggt

SEQ ID NO: 114 - RNA, 24 nt, synthetic construct
  source 1..24: mol_type = other RNA
gcggtctaggtgccctcgttcggt

SEQ ID NO: 115 - RNA, 24 nt, synthetic construct
  source 1..24: mol_type = other RNA
gcggtctaggtgccctcgtagcct